(19) United States
(12) Patent Application Publication
(10) Pub. No.: US 2023/0011134 A1
(43) Pub. Date: Jan. 12, 2023

(54) COMPOSITIONS AND METHODS FOR THE TREATMENT OF PROSTATE CANCER

(71) Applicant: Janssen Biotech, Inc., Horsham, PA (US)

(72) Inventors: Rhys Salter, Doylestown, PA (US); Neha Saroj Saxena, Philadelphia, PA (US); Chaitanya Divgi, Spring House, PA (US); Joseph O'Donoghue, Spring House, PA (US)

(21) Appl. No.: 17/752,892

(22) Filed: May 25, 2022

Related U.S. Application Data
(60) Provisional application No. 63/193,704, filed on May 27, 2021.
(60) Provisional application No. 63/335,761, filed on Apr. 28, 2022.

(51) Int. Cl. A61K 51/10; A61K 47/22; A61K 47/12; A61K 47/26; A61P 35/00

(52) U.S. Cl. CPC A61K 51/1096; A61K 47/22; A61K 47/12; A61K 47/26; A61P 35/00

(57) ABSTRACT

Embodiments of the present invention provide compositions and methods for the treatment of cancer, in particular prostate cancer. According to certain embodiments, a method of treating cancer in a patient comprises administering to the patient a therapeutically effective amount of a radioconjugate, wherein the radioconjugate comprises an antibody or antigen binding domain with binding specificity for hK2. Also provided herein are pharmaceutical compositions comprising radiolabeled antibodies with binding specificity for hK2.

### CROSS-REFERENCE TO RELATED APPLICATIONS

This application claims the benefit of priority of U.S. Provisional Patent Application No. 63/193,704, filed May 27, 2021, and U.S. Provisional Patent Application No. 63/335,761, filed Apr. 28, 2022, which are incorporated by reference herein, in their entireties and for all purposes.

### TECHNICAL FIELD

Embodiments of the present invention relate to compositions and methods for the treatment of prostate cancer. In particular, embodiments of the present invention relate to radioconjugate compositions for hK2-targeted therapies.

### REFERENCE TO SEQUENCE LISTING SUBMITTED ELECTRONICALLY

This application contains a sequence listing, which is submitted electronically via EFS-Web as an ASCII formatted sequence listing with a file name JBI6423USNP1_SeqListing.txt” and a creation date of May 12, 2022, and having a size of 17 kb. The sequence listing submitted via EFS-Web is part of the specification and is herein incorporated by reference in its entirety.

### BACKGROUND

Prostate cancer is one of the most common forms of cancer. The growth of the tumor is usually a process that takes place over a long period of time. Prostate cancer is often a mild form of cancer. In fact, the majority of people diagnosed with prostate cancer survive and recover. A minority of people encounter a more aggressive form of prostate cancer, which metastasizes in an early stage. This aggressive form of prostate cancer may only be curable if it is diagnosed at an early stage, before the cancer has spread to extracapsular tissue.

The landscape for the management of patients with metastatic castration-resistant prostate cancer (mCRPC) has changed with the approval of several new agents, including androgen receptor-(AR) directed therapy (e.g., enzalutamide and abiraterone acetate plus prednisone), chemotherapy (e.g., docetaxel and cabazitaxel), and cellular immune therapy (e.g., Sipuleucel-T). With these agents, overall survival has improved from the previously reported range of 6 to 10 months to 18 to 24 months. However, most prostate cancer patients will experience disease progression on anti-androgen or androgen synthesis inhibitor therapy within 13 to 20 months.

There remains a need for new therapeutic agents and methods for treating and diagnosing prostate cancer; in particular, therapies with mechanisms of action that overcome pathways of resistance are crucial in developing alternative strategies for the treatment of mCRPC.

### SUMMARY

The present invention relates to pharmaceutical compositions, methods of making pharmaceutical compositions, and methods of treating cancer in a patient in need of such treatment.

According to an embodiment of the present invention, a method of treating cancer in a patient comprises: administering to the patient a therapeutically effective amount of a pharmaceutical composition comprising a radioconjugate and one or more pharmaceutically acceptable excipients, wherein: the radioconjugate comprises at least one radiometal complex conjugated to an antibody, or an antigen binding fragment, with binding specificity for hK2; the radiometal complex comprises a radiometal; and the radiometal provides a targeted radioactivity from about 50 μCi to about 350 μCi per dose of the pharmaceutical composition at the time of dosing.

According to an embodiment of the present invention, a method of treating cancer in a patient comprises: administering to the patient a therapeutically effective amount of a pharmaceutical composition comprising a radioconjugate and one or more pharmaceutically acceptable excipients, wherein: the radioconjugate comprises at least one radiometal complex conjugated to an antibody with binding specificity for hK2; the radiometal complex comprises a radiometal that is ²²⁵Ac; and the radiometal provides a targeted radioactivity from about 50 μCi to about 350 μCi per dose of the pharmaceutical composition at the time of dosing.

According to an embodiment, the radioconjugate comprises at least one radiometal complex conjugated to an antibody with binding specificity for hK2, wherein the antibody comprises a heavy chain variable region comprising the amino acid sequences of SEQ ID NO: 1 and SEQ ID NO:2 and SEQ ID NO:3; and a light chain variable region comprising the amino acid sequences of SEQ ID NO:4 and SEQ ID NO:5 and SEQ ID NO:6.

According to an embodiment, the radiometal complex comprises a chelator that is DOTA.

According to an embodiment, the radioconjugate comprises the radiometal chelated to (a) a compound of formula (IV)

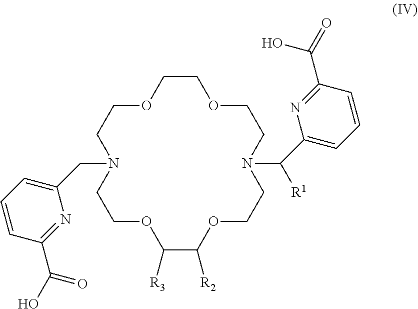

or a pharmaceutically acceptable salt thereof, wherein:
   R₁ is hydrogen and R₂ is -L₁-R₄;
   alternatively, R₁ is -L₁-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L₁-R₄,
   L₁ is absent or a linker; and
   R₄ is the antibody; or
(b) a compound of formula (V)

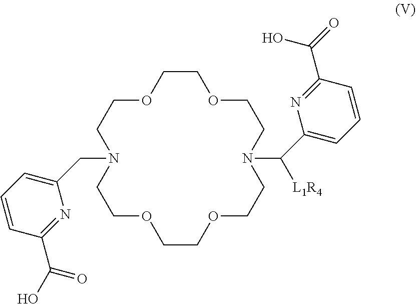

or a pharmaceutically acceptable salt thereof, wherein:
   L₁ is absent or a linker; and
   R₄ is the antibody;
for example, wherein the chelator is a compound of the following formula or a pharmaceutically acceptable salt thereof:

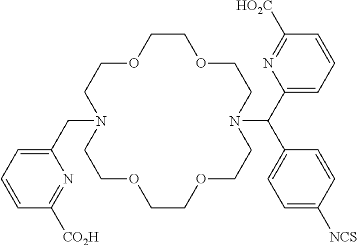

According to an embodiment, the radiometal is ²²⁵Ac and the radiometal provides a targeted specific activity from about 25 μCi to about 350 μCi per about 2 mg of total antibody, or from about 50 μCi to about 350 μCi per about 2 mg of total antibody.

According to an embodiment, the method comprises administering the pharmaceutical composition to the patient intravenously.

Embodiments of the present invention are particularly useful in treating patients that have been diagnosed with prostate cancer; for example, patients that have late-stage prostate cancer. According to an embodiment, the cancer is non-localized prostate cancer. According to another embodiment, the cancer is metastatic prostate cancer. According to another embodiment, the cancer is castration-resistant prostate cancer (CRPC). According to another embodiment, the cancer is metastatic castration-resistant prostate cancer (mCRPC). According to another embodiment, the cancer is mCRPC with adenocarcinoma.

Another embodiment of the present invention provides a pharmaceutical composition comprising a radioconjugate and one or more pharmaceutically acceptable excipients, wherein: the radioconjugate comprises at least one radiometal complex conjugated to an antibody, or an antigen binding fragment, with binding specificity for hK2, and the radiometal complex comprises a radiometal.

According to an embodiment, the pharmaceutical composition comprises a radioconjugate and one or more pharmaceutically acceptable excipients, wherein: the radioconjugate comprises at least one radiometal complex conjugated to an antibody with binding specificity for hK2, the radiometal complex comprises a radiometal that is ²²⁵Ac, and the antibody comprises a heavy chain variable region comprising the amino acid sequences of SEQ ID NO:1 and SEQ ID NO:2 and SEQ ID NO:3; and a light chain variable region comprising the amino acid sequences of SEQ ID NO:4 and SEQ ID NO:5 and SEQ ID NO:6.

According to an embodiment, the one or more pharmaceutically acceptable excipients comprise one or more radioprotectants, such as sodium ascorbate, gentisic acid, or a combination thereof (e.g., in an amount of about 0.1 to about 5 w/v %, or about 0.1 to about 4 w/v %, or about 0.1 to about 3 w/v %, about 0.1 to about 2 w/v %, or about 0.1 to about 1 w/v %, or about 0.25 to about 0.75 w/v %, or about 0.5 w/v %).

According to an embodiment, the one or more pharmaceutically acceptable excipients comprise one or more surfactants, such as polysorbate 20.

According to an embodiment, the pharmaceutical composition comprises the radioconjugate, sodium ascorbate, polysorbate 20, acetate buffer and water.

According to an embodiment, the pharmaceutical composition comprises the radioconjugate, about 24-28 mM acetate, about 0.25-0.75 w/v % sodium ascorbate, and about 0.01-0.15 w/v % polysorbate 20 in water.

According to an embodiment, the pharmaceutical composition has a pH from about 5 to about 6 (e.g., about 5.5).

According to an embodiment, the pharmaceutical composition does not contain any cryoprotectants, such as sugars or sugar alcohols.

According to an embodiment, the radiometal is ²²⁵Ac and the radiometal provides a specific activity from about 50 μCi to about 350 μCi per about 2 mg of total antibody at the time of dosing.

According to an embodiment, the pharmaceutical composition comprises a total amount of conjugate intermediate and radioconjugate in an amount of about 0.1-1.0 mg/mL; for example, about 0.5 mg/mL.

Another embodiment of the present invention provides a method of making the pharmaceutical composition comprising combining a first intermediate composition and a second intermediate composition to form the pharmaceutical composition, wherein: the first intermediate composition comprises the radioconjugate, and the second intermediate composition comprises a conjugate intermediate and does not contain any radioconjugate.

### BRIEF DESCRIPTION OF THE DRAWINGS

the following drawings form part of the present specification and are included to further demonstrate certain aspects of the present invention. The invention may be better understood by reference to one or more of these drawings in combination with the description of specific embodiments presented herein.

FIGS. 1A and 1B are high performance liquid chromatographs of a drug product composition (FIG. 1A) and ¹¹¹In-DOTA-h11B6 (FIG. 1B).

FIGS. 2A and 2B are high performance liquid chromatographs of an ²²⁵Ac-DOTA-h11B6 drug product comprising sucrose and lacking ascorbate at T=0 (FIG. 2A) and T=96 h (FIG. 2A).

FIGS. 3A-3D are high performance liquid chromatographs for a drug product. The compositions comprise about 50 μCi (FIGS. 3A and 3B) and 200 μCi of ²²⁵Ac-DOTA-h11B6

(FIGS. 3C and 3D) per 4 mL of drug product. The compositions of FIGS. 3A and 3C further comprise sodium ascorbate and the compositions of FIGS. 3B and 3D further comprise sucrose.

FIGS. 4A and 4B illustrate examples of radioconjugates of the present invention (bonds between Ac-225 and the chelators are not shown).

FIG. 5A provides an illustration of a conjugate intermediate comprising DOTA, in which the lysine portion is not shown. FIG. 5B provides an illustration of a conjugate intermediate comprising DOTA, in which the lysine portion is represented. FIG. 5C provides an illustration of a conjugation process for a conjugate intermediate comprising DOTA.

FIG. 6A provides an illustration of a conjugate intermediate comprising a TOPA chelator (TOPA-h11B6), in which the lysine portion is not shown. FIG. 6B provides an illustration of the conjugate intermediate shown in FIG. 6A, in which the lysine portion is represented. FIG. 6C provides an illustration of a conjugation process for the conjugate intermediate.

FIG. 7 shows the amino acid sequences for bulk heavy and light chains of an h11B6 antibody.

### DETAILED DESCRIPTION OF ILLUSTRATIVE EMBODIMENTS

The compounds, compositions and methods described herein are useful in treating cancer in a patient in need of such treatment. Embodiments of the compounds, compositions and methods are effective in treating prostate cancer, including advanced stages of prostate cancer, particularly castration-resistant prostate cancer (CRPC) and, thus, result in longer survival rates for patients. These compounds are particularly useful in patients where existing treatments for advanced stages of prostate are deemed unsuccessful.

Human kallikrein 2 (hK2) is a trypsin-like antigen produced by columnar prostate epithelial cells and driven by androgen receptor (AR) signaling in a manner identical to the closely related prostate-specific antigen (PSA; human glandular kallikrein 3) with which genetically it has an 80% homology with the PSA gene. Unlike PSA however, circulating levels of hK2 are found at exceptionally low levels, where they can be bound by multiple protease inhibitor complexes. Although hK2 is believed to be primarily secreted, there is evidence that it is capable of inducing internalization via an antigen-antibody complex hence it is believed to exist on the cell surface as well. Because hK2 expression is highly specific for prostate adenocarcinoma and increases throughout disease progression, hK2-targeted therapies are attractive.

An exemplary hK2 sequence is described as Transcript: KLK2-201 (ENST00000325321), provided herein as SEQ ID NO:7, a product of gene ENSG00000167751, as given in the ensemble database.

### Certain Terminology

“Antigen binding fragment” or “antigen binding domain” refers to a portion of an isolated protein that binds an antigen. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multi specific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a hK2 variant may result from one or more (such as, for example, about 1 to about 25, about 1 to about 20, about 1 to about 15, about 1 to about 10, or about 1 to about 5) changes to an amino acid sequence of a native hK2. Also by way of example, a variant of an anti-hK2 antibody, such as h11B6, may result from one or more (such as, for example, about 1 to about 25, about 1 to about 20, about 1 to about 15, about 1 to about 10, or about 1 to about 5) changes to an amino acid sequence of a native or previously unmodified anti-hK2 antibody. Variants may be naturally occurring, such as allelic or splice variants, or may be artificially constructed. Polypeptide variants may be prepared from the corresponding nucleic acid molecules encoding the variants. In specific embodiments, the hK2 variant or anti-hK2 antibody variant at least retains hK2 or anti-hK2 antibody functional activity, respectively. In specific embodiments, an anti-hK2 antibody variant binds hK2 and/or is antagonistic to hK2 activity. In certain embodiments, the variant is encoded by a single nucleotide polymorphism (SNP) variant of a nucleic acid molecule that encodes hK2 or anti-hK2 antibody VH or VL regions or subregions, such as one or more CDRs.

A non-limiting example of a variant when used in relation to an antibody is an “Fc variant” which is an antibody having a variant Fc region. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith

The term “identity” refers to a relationship between the sequences of two or more polypeptide molecules or two or more nucleic acid molecules, as determined by aligning and comparing the sequences. “Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

“Specifically binds,” “specific binding,” “specifically binding” or “binds” refer to a protein molecule binding to an antigen or an epitope within the antigen with greater affinity than for other antigens. Typically, the protein molecule binds to the antigen or the epitope within the antigen with an equilibrium dissociation constant (K_{D}) of about 1×10⁻⁷ M or less, for example about 5×10⁻⁸M or less, about 1×10⁻⁸M or less, about 1×10⁻⁹M or less, about 1×10⁻¹⁰ M or less, about 1×10⁻¹¹M or less, or about 1×10¹²M or less, typically with the K_{D} that is at least one hundred fold less than its K_{D} for binding to a non-specific antigen (e.g., BSA, casein). As used herein, an antibody or antigen binding domain “with binding specificity for hK2” refers to an antibody or antigen binding domain that specifically binds to hK2, respectively.

As used herein, in certain embodiments, the term “subject” refers to a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder. The term “patient” as used herein refers to a human.

“Administer” or “administration” refers to the act of physically delivering a substance as it exists outside the body into a patient, by injection or otherwise, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The term “effective amount” or “therapeutically effective amount” as used herein refers to the amount of a radioconjugate or a pharmaceutical composition provided herein which is sufficient to result in the desired therapeutic effect for a given condition and administration regimen.

The terms medicament, pharmaceutical, active agent, active pharmaceutical ingredient (API), drug, medication, and active are used herein interchangeably to refer to the pharmaceutically active compound(s) in a pharmaceutical composition. An example of an API suitable for use in accordance with the present invention is a radioconjugate with binding specificity for hK2. A pharmaceutical composition may include one or more API(s) and one or more additional ingredients referred to herein as “excipients.” Preferably, the excipients are substantially or completely pharmaceutically inert.

The term “dose” refers to the total amount of a particular pharmaceutical composition administered to a patient at a particular time. Preferably, a dose is delivered as a single administration of a unit dose of pharmaceutical composition (e.g., via an intravenous administration). Alternatively, there may be multiple administrations of a unit dose that has been subdivided into multiple sub-doses (wherein a sub-dose refers to a portion of the unit dose).

The term “pharmaceutically acceptable,” as used herein, means non-toxic and preferably permitted by a regulatory agency, e.g., of a European or U.S. Federal or state government, or listed in the U.S. Pharmacopeia or other generally recognized pharmacopeia for use in animals, and more particularly in humans.

Radioactive decay refers to the process by which an unstable atomic nucleus loses energy by radiation to produce at least one daughter nuclide. Half-life refers to the time required for one half of the atomic nuclei of a radioactive sample to decay to its daughter nuclide. The non-SI unit of measure for radioactivity of a substance is curie (Ci). One curie is equal to that quantity of radioactive material in which the number of atoms decaying per second is equal to 37 billion (3.7×10¹⁰). An alternative unit of measure for the radioactivity of a substance is the SI unit of the becquerel (Bq). The becquerel is equal to that quantity of radioactive material in which one atomic nucleus decays per second. Specific activity refers to the amount of radioactivity per unit mols or mass in a sample; for example, sometimes expressed as Ci/mmol or Ci/mg. Radioactive concentration, also known as specific concentration (e.g., expressed as mCi/mL or μCi/mL) refers to the total amount of radioactivity per unit volume.

In reference to immunoconjugates and radioconjugates, the term “conjugated” means “joined.” Molecules (such as an antibody and a chelator) may be joined to each other, for example, by covalent bonding.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The transitional term “comprising” is intended to connote its generally accepted meaning in the patent vernacular. “Comprising,” is synonymous with “including” or “containing” and is inclusive or open-ended and does not exclude additional, unrecited elements or method steps.

The term “between” as used in a phrase as such “between A and B” or “between A-B” refers to a range including both A and B. The term “from” as used in a phrase as such “from A to B” or “from A-B” refers to a range including both A and B.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range. In certain embodiments, the term “about” signifies a variance of ±10% of the associated value, and additional embodiments include those where the variance may be ±5%, ±15%, ±20%, ±25%, or ±50%.

It is to be appreciated that certain features of the present invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Abbreviations utilized through this disclosure include:

| | |
|---|---|
| ²²⁵Ac | actinium-225 |
| ¹¹¹In | indium-111 |
| α | alpha |
| AE | adverse event |
| ALP | alkaline phosphatase |
| ALT | alanine aminotransferase |
| AR | androgen receptor |
| AST | aspartate aminotransferase |
| AUC₀₋ₜ | area under the serum concentration-time curve from time |
| | zero to t time |
| BARAC | biarylazacyclooctynonyl |
| BCN | bicyclononynyl |
| BLRM | bayesian logistic regression model |
| Bn | benzyl |
| Cₘₐₓ | maximum observed serum concentration/radioactivity |
| CRPC | castration-resistant prostate cancer |
| DIFO | difluorinated cyclooctynyl |
| DIBAC | dibenzoazacyclooctynyl |
| DIBO | dibenzocyclooctynyl |
| DIFBO | difluorobenzocyclooctynyl |
| DIMAC | dimethoxyazacyclooctynyl |
| DLT | dose-limiting toxicity |
| DO3A | 5-S-(4-aminobenzyl)-l-oxa-4,7,10-triazacyclododecane- |
| | 4,7,10-tris(acetic acid) |
| DOTA | 1,4,7,10-tetraazacyclododecane-1,4,7,10,tetraacetic acid |
| DRE | digital rectal exam |
| ECOG | Eastern Cooperative Oncology Group |
| EWOC | escalation with overdose control |
| GGT | gamma-glutamyl transferase |
| GnRH | gonadotropin-releasing hormone |
| hK2 | human kallikrein-2 |
| LHRH | luteinizing hormone-releasing hormone |
| mCRPC | metastatic castration-resistant prostate cancer |
| mCRM | modified continual reassessment method |
| NCI CTCAE | National Cancer Institute Common Terminology Criteria |
| | for Adverse Events |
| NOTA | S-2-(4-isothiocyanatobenzyl)-l,4,7-triazacyclononane- |
| | l,4,7-triacetic acid |
| ORR | overall response rate |
| PCTA | 3,6,9,15-tetraazabicyclo[9.3.1]-pentadeca-1(15),11,13- |
| | triene-4-(S)-(4-isothiocyanatobenzyl)-3,6,9-triacetic acid |
| PCWG3 | Prostate Cancer Working Group 3 |
| PR | Partial Response |
| RECIST | Response Evaluation Criteria in Solid Tumors |
| RP2D | recommended phase 2 dose(s) |
| PCL | poly(caprolactone) |
| PEG | polyethylene glycol |
| PGA | poly(glycolic acid) |
| PLA | poly(lactic acid) |
| PLGA | copolymers of PLA and PGA |
| PSA | prostate-specific antigen |
| TETA | 1,4,8,11-tetraazacyclodocedan-l,4,8,11-tetraacetic acid |
| Tₘₐₓ | time to reach maximum observed serum concentration/ |
| | radioactivity |
| ULN | upper limit of normal |
| MOBO | monobenzocyclooctynyl |
| TMDIBO | tetramethoxy DIBO |

The present invention may be understood more readily by reference to the following description taken in connection with the accompanying drawing and examples, all of which form a part of this disclosure. It is to be understood that the present invention is not limited to the specific compounds, methods, conditions or parameters described or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement.

### Radioconjugates of the Present Invention

Embodiments of the present invention relate to compositions and methods for targeting hK2 with a radioconjugate to achieve efficacious cancer cell death (e.g., tumor cell death) in prostate cancer patients. As used herein, an “immunoconjugate” refers to an antibody, or an antigen binding domain, that is conjugated (joined, e.g., bound via a covalent bond) to a second molecule, such as a toxin, drug, radiometal ion, chelator, radiometal complex, etc. A “radioconjugate” (also referred to herein as a “radioimmunoconjugate”) in particular refers to an antibody, or an antigen binding domain, that is conjugated (joined, e.g., bound via a covalent bond) to at least one radiometal complex. Stated another way, a radioconjugate refers to at least one radiometal complex joined, e.g., bound via a covalent bond, to an antibody or antigen binding domain. A radioconjugate may comprise at least one radiometal complex that comprises a linker, wherein the radiometal complex is joined to the antibody or antigen binding domain via the linker.

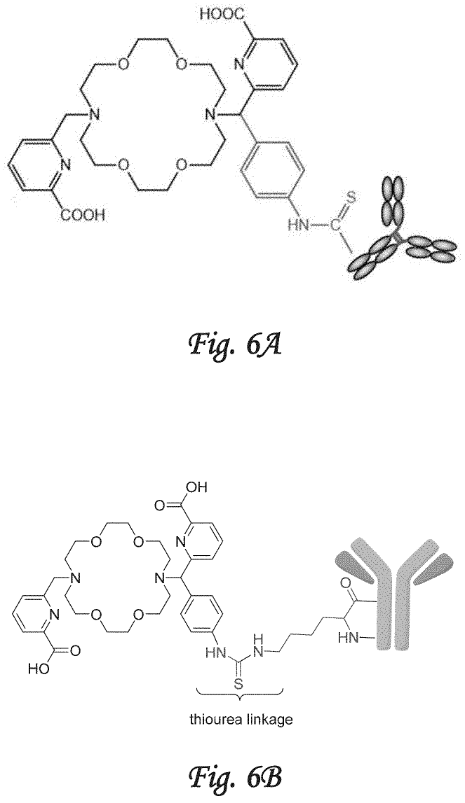

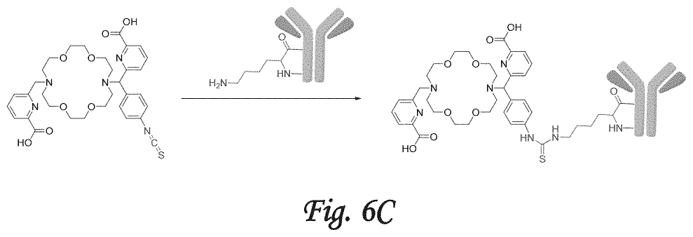

As used herein, an “antibody-chelator complex” or “conjugate intermediate” or “drug substance intermediate” refers to a precursor of a radioconjugate, which comprises an antibody, or antigen binding domain, that is conjugated (joined, e.g., bound via a covalent bond) to a chelator that does not comprise a radiometal. A conjugate intermediate may comprise a linker, wherein the chelator is joined to the antibody or antigen binding domain via the linker. After a radiometal is chelated to the chelator of a conjugate intermediate, it becomes a radioconjugate. For example, “DOTA-mAb” refers to a conjugate intermediate comprising an DOTA conjugated to an antibody. An example of a conjugate intermediate is DOTA-h11B6. As used herein, “DOTA-h11B6” is a conjugate intermediate which comprises DOTA conjugated to h11B6, optionally via a linker. Non-limiting examples of DOTA-mAbs are illustrated in FIGS. 5A-5C. Another example of a conjugate intermediate is TOPA-h11B6. As used herein, “TOPA-h11B6” is a conjugate intermediate which comprises TOPA conjugated to h11B6, optionally via a linker. Non-limiting examples of TOPA-mAbs are illustrated in FIGS. 6A-6C.

A chelator may be conjugated to an antibody according to methods known in the art; for example, a chelator may be conjugated to an antibody via a linker. Thus, radioconjugates and conjugate intermediates of the present invention may comprise a chelator joined to an antibody by a linker. As used herein, the term linker generally refers to a chemical moiety that joins a chelator to an antibody or antigen binding domain. Any suitable linker known to those skilled in the art in view of the present disclosure can be used in the invention. The linkers can contain, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB).

According to certain embodiments, a chelator or chelator-linker comprises a nucleophilic moiety or an electrophilic moiety, as described herein. Reaction of the nucleophilic group or electrophilic group of a chelator or chelator-linker with an antibody or antigen binding domain comprising the corresponding reaction partner allows for covalent linkage of the antibody or antigen binding domain to the chelator-linker. As used herein with respect to compounds of Formulae (I), (II), (III), (IV), (V) and (VI), a linker (L₁) may be joined to an electrophilic moiety or a nucleophilic moiety (R₁₁) to form is -L₁-R₁₁. Examples of nucleophilic groups include, but are not limited to, azides, amines, and thiols. Examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups include, but are not limited to, Michael acceptors (e.g., maleimide), haloacetyl, acyl halides, activated disulfides, and phenyloxadiazole sulfone.

According to certain embodiments, a conjugation reaction results in addition of one or multiple chelator molecules (e.g., DOTA molecules) to the epsilon amino group of lysine side chains of an antibody (e.g., an h11B6 mAb). For example, 1, 2, 3, 4 or 5 DOTA molecules may be conjugated to an antibody. According to certain embodiments, p-SCN-Bn-DOTA may be reacted with an antibody to form a conjugate intermediate comprising DOTA, as illustrated in FIGS. 5A and 5B. FIG. 5A provides an illustration of a conjugate intermediate comprising DOTA, in which the lysine portion is not shown. FIG. 5B provides an illustration of a conjugate intermediate comprising DOTA, in which the lysine portion is represented. FIGS. 6B and 6C provide illustrations of a conjugate intermediate comprising an alternative chelator in accordance with the present invention.

The chelator-to-antibody ratio (CAR), which designates the number of chelator-linker molecules per antibody molecule, can be measured by intact mass analysis using RP-HPLC with online mass analysis. According to certain embodiments, the average CAR of a conjugate intermediate of the present invention (e.g., a DOTA-mAb, such as DOTA-h11B6) is from about 1 to about 8, of from about 1 to about 7, or from about 1 to about 6, or from about 1 to about 5, or from about 1 to about 4, or from about 1 to about 3, or from about 2 to about 4, or from about 2 to about 3.

According to particular embodiments, a radioconjugate described herein comprises a radiometal complex conjugated to an antibody, or an antigen binding domain, with binding specificity for kallikrein related peptidase 2 (hK2). According to particular embodiments, the radioconjugate is a radiolabeled antibody, which comprises an antibody conjugated (joined) to a radiometal complex. According to particular embodiments, the radioconjugate comprises an antibody, such as h11B6, conjugated to a radiometal complex comprising a chelator and a radiometal. In some embodiments, the antibody is covalently bound to the chelator. As described herein, the radiometal complex optionally comprises a linker.

A “radiometal complex” as used herein refers to a complex comprising a radiometal ion associated with a chelator that is a macrocyclic compound. Typically, a radiometal ion is bound to or coordinated to a macrocyclic compound via coordinate bonding. Heteroatoms of the macrocyclic ring can participate in coordinate bonding of a radiometal ion to a macrocycle compound. A macrocycle compound can be substituted with one or more substituent groups, and the one or more substituent groups can also participate in coordinate bonding of a radiometal ion to a macrocycle compound in addition to, or alternatively to the heteroatoms of the macrocyclic ring. Other examples of possible linkages between the chelator and radioisotope include guest-hosting binding such as ionic bonding, hydrogen bonding, van der Waals forces or hydrophobic interactions. A radiometal complex may optionally comprise a linker, which is a chemical moiety that joins the chelator to the antibody or antigen binding domain.

As used herein, the terms “radiometal,” “radioisotope,” “radiometal ion” and “radioactive metal ion” are used interchangeably and refer to one or more isotopes of the elements that emit particles and/or photons. Non-limiting examples of radioisotopes that may be used for therapeutic applications in accordance with the present invention include, e.g., beta or alpha emitters, such as, e.g., ²²⁵Ac, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th. Other non-limiting examples of radioisotopes that may be used as imaging agents in accordance with the present invention include gamma-emitting radioisotopes, such as, e.g., ¹⁷⁷Lu, ⁶²Cu, ⁶⁴Cu, ⁶⁷Ga, ⁶⁸Ga, ⁸⁶Y, ⁸⁹Zr, and ¹¹¹In. In certain embodiments, the radiometal ion is a “therapeutic emitter,” meaning a radiometal ion that is useful in therapeutic applications. Examples of therapeutic emitters include, but are not limited to, beta or alpha emitters, such as, ¹³²La, ¹³⁵La, ¹³⁴Ce, ¹⁴⁴Nd, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²²⁵Ac, ²⁵⁵Fm and ²²⁷Th, ²²⁶Th, ²³⁰U. Preferably, a radiometal ion used in the invention is an alpha-emitting radiometal ion, such as actinium-225 (²²⁵Ac).

It is noted that certain radiometals may be used as therapeutic agents (e.g., ²²⁵Ac) and/or as imaging agents (e.g., ¹¹¹In) A suitable radiometal for use as a therapeutic agent is one that is capable of reducing or inhibiting the growth of, or in particular killing, a cancer cell, such as a prostate cancer cell. In certain embodiments, radioconjugates of the present invention can deliver a cytotoxic payload with the ability to emit alpha and/or beta particles in the vicinity of a tumor by binding onto cancer cells' surface antigens and initiating cell death. In certain embodiments, the radioconjugates of the present invention are internalized into hk2-expressing cancer cells.

The term “225Ac,”, “²²⁵Ac,” or “Ac-225” as used herein refer to actinium-225 which is an alpha-emitting radiometal. According to particular embodiments, the approximate ten-day half-life of ²²⁵Ac (about 9.9 days) is long enough to be able to prepare the compounds described herein, but short enough to match the circulation pharmacokinetics of the antibody that is conjugated to the radiometal complex, such as h11B6. ²²⁵Ac decays in a series of steps that ultimately emits four alpha particles before reaching a stable isotope, ²⁰⁹Bi, thereby providing an increased potency of the compounds.

### Antibodies of the Present Invention

According to particular embodiments, the radioconjugate of the present invention comprises an antibody that is an h11B6 antibody. Embodiments of an h11B6 antibody are described in U.S. Pat. No. 10,100,125, which is incorporated by reference herein. As used herein, an “h11B6 antibody” or “h11B6 mAb” or “h11B6” or “hu11B6” refers to an antibody with binding specificity for human kallikrein-2 (hK2), wherein the antibody comprises (a) a heavy chain variable region comprising the amino acid sequences of SEQ ID NO:1 and SEQ ID NO:2 and SEQ ID NO:3; and/or (b) a light chain variable region comprising the amino acid sequences of SEQ ID NO:4 and SEQ ID NO:5 and SEQ ID NO:6, wherein the heavy chain variable region and light chain variable region comprise framework amino acid sequences from one or more human antibodies.

According to particular embodiments, the radioconjugate of the present invention comprises an h11B6 antibody which comprises (a) a heavy chain variable region (VH) comprising a VH CDR1 having an amino acid sequence of SEQ ID NO:1 (SDYAWN), a VH CDR2 having an amino acid sequence of and SEQ ID NO:2 (YISYSGSTTYNPSLKS) and a VH CDR3 having an amino acid sequence of SEQ ID NO:3 (GYYYGSGF); and (b) a light chain variable region (VL) comprising a VL CDR1 having an amino acid sequence of SEQ ID NO:4 (KASESVEYFGTSLMH), a VL CDR2 having an amino acid sequence of and SEQ ID NO:5 (AASNRES) and a VL CDR3 having an amino acid sequence of SEQ ID NO:6 (QQTRKVPYT).

The above six amino acid sequences represent the complementarity-determining regions (CDRs), as defined according to Kabat et al., (1991) Sequences of Immunological Interest, 5th edition, NIH, Bethesda, Md. (the disclosures of which are incorporated herein by reference). Kabat numbering scheme (Kabat et al., 1991) is used throughout this description.

According to particular embodiments, the radioconjugate of the present invention comprises an h11B6 antibody which comprises a heavy chain variable region (VH) having at least 80%, at least 85%, at least 90%, at least 95%, or at least 98% sequence identity to the amino acid sequence of SEQ ID NO: 8, and/or a light chain variable region (VL) having at least 80%, at least 85%, at least 90%, at least 95%, or at least 98% sequence identity to the amino acid sequence of SEQ ID NO: 9.

According to particular embodiments, the radioconjugate of the present invention comprises an h11B6 antibody which comprises a heavy chain variable region (VH) comprising the amino acid sequence of SEQ ID NO: 8, and/or a light chain variable region (VL) comprising the amino acid sequence of SEQ ID NO: 9.

SEQ ID NO: 8 is the following:

QVQLQESGPGLVKPSDTLSLTCAVSGNSITSDYAWNWIRQPPGKG
LEWIGYISYSGSTTYNPSLKSRVTMSRDTSKNQFSLKLSSVTAVD
TAVYYCATGYYYGSGFWGQGTLVTVSS

SEQ ID NO 9 is the following:

DIVLTQSPDSLAVSLGERATINCKASESVEYFGTSLMHWYQQKP
GQPPKLLIYAASNRESGVPDRFSGSGSGTDFTLTISSLQAEDVAV
YYCQQTRKVPYTFGQGTKLEIK

According to particular embodiments, the radioconjugate of the present invention comprises an h11B6 antibody which comprises a heavy chain constant region having at least 80%, at least 85%, at least 90%, at least 95%, or at least 98% sequence identity to the amino acid sequence of SEQ ID NO: 10, and/or a light chain constant region having at least 80%, at least 85%, at least 90%, at least 95%, or at least 98% sequence identity to the amino acid sequence of SEQ ID NO: 11.

According to particular embodiments, the radioconjugate of the present invention comprises an h11B6 antibody which comprises a heavy chain constant region comprising the amino acid sequence of SEQ ID NO: 10, and/or a light chain constant region comprising the amino acid sequence of SEQ ID NO: 11.

SEQ ID NO: 10 is the following:

ASTKGPSVFPLAPSSKSTSGGTAALGCLVKDYFPEPVTVSWNS
GALTSGVHTFPAVLQSSGLYSLSSVVTVPSSSLGTQTYICNVNH
KPSNTKVDKKVEPKSCDKTHTCPPCPAPELLGGPSVFLFPPKPK
DTLMISRTPEVTCVVVDVSHEDPEVKFNWYVDGVEVHNAKTK
PREEQYNSTYRVVSVLTVLHQDWLNGKEYKCKVSNKALPAPI
EKTISKAKGQPREPQVYTLPPSREEMTKNQVSLTCLVKGFYPS
DIAVEWESNGQPENNYKTTPPVLDSDGSFFLYSKLTVDKSRWQ
QGNVFSCSVMHEALHNHYTQKSLSLSPGK

SEQ ID NO: 11 is the following:

RTVAAPSVFIFPPSDEQLKSGTASVVCLLNNFYPREAKVQWKV
DNALQSGNSQESVTEQDSKDSTYSLSSTLTLSKADYEKHKVYA
CEVTHQGLSSPVTKSFNRGEC

According to particular embodiments, the radioconjugate of the present invention comprises an h11B6 antibody which comprises a heavy chain having at least 80%, at least 85%, at least 90%, at least 95%, or at least 98% sequence identity to the amino acid sequence of SEQ ID NO: 12, and/or a light chain having at least 80%, at least 85%, at least 90%, at least 95%, or at least 98% sequence identity to the amino acid sequence of SEQ ID NO: 13.

According to particular embodiments, the radioconjugate of the present invention comprises an h11B6 antibody which comprises a heavy chain having the amino acid sequence of SEQ ID NO: 12, and/or a light chain having the amino acid sequence of SEQ ID NO: 13.

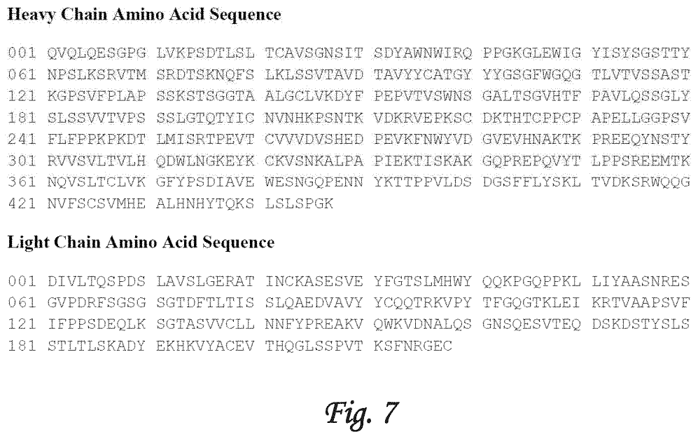

The amino acid sequence for the h11B6 heavy and light chains is also shown in FIG. 7.

According to particular embodiments, an antibody of the present invention (e.g., h11B6) comprises or consists of an intact (i.e. complete) antibody, such as an IgA, IgD, IgE, IgG or IgM molecule.

According to particular embodiments, an antibody of the present invention (e.g., h11B6) comprises or consists of an intact IgG molecule, or a variant of the same. The IgG molecule may be of any known subtype, for example IgG1, IgG2, IgG3 or IgG4.

According to particular embodiments, the radioconjugate of the present invention comprises an h11B6 antibody that is an IgG1 antibody. According to particular embodiments, the radioconjugate of the present invention comprises an h11B6 antibody that is an IgG1 kappa isotype. According to particular embodiments, the radioconjugate of the present invention comprises an h11B6 antibody that is an IgG1 antibody or a variant thereof, such as an Fc variant.

According to an embodiment, the radioconjugate of the present invention comprises an antibody conjugated to DOTA, optionally via a linker. For example, the h11B6 antibody may be conjugated to DOTA to produce a DOTA-h11B6 conjugate intermediate, and DOTA-h11B6 is then chelated to ²²⁵Ac to produce the radioconjugate ²²⁵Ac-DOTA-h11B6.

According to certain embodiments, DOTA-h11B6 is formed by chemically conjugating h11B6 to a DOTA derivative, p-SCN-Bn-DOTA (CAS Registry Number: 127985-74-4; Chemical Name: 2-S-(4-Isothiocyanatobenzyl)-1,4,7,10-tetraazacyclododecane-1,4,7,10-tetraacetic acid) according to known methods, resulting in addition of multiple DOTA molecules to the epsilon amino group of lysine side chains of the h11B6 mAb. DOTA-h11B6 may then be chelated to ²²⁵Ac to produce the radioconjugate ²²⁵Ac-DOTA-h11B6. When administered to a patient, the radioconjugate ²²⁵Ac-DOTA-h11B6 can bind and internalize within hK2-expressing cells.

According to an embodiment, the radioconjugate of the present invention comprises an antibody conjugated to TOPA, optionally via a linker. For example, the h11B6 antibody may be conjugated to TOPA to produce a TOPA-h11B6 conjugate intermediate, and TOPA-h11B6 is then chelated to ²²⁵AC to produce the radioconjugate ²²⁵Ac-TOPA-h11B6.

According to certain embodiments, TOPA-h11B6 is formed as described in WO 2020/229974 or PCT/IB2021/060350. TOPA-h11B6 may then be chelated to²²⁵AC to produce the radioconjugate ²²⁵Ac-TOPA-h11B6. When administered to a patient, the radioconjugate ²²⁵Ac-TOPA-h11B6 can bind and internalize within hK2-expressing cells.

According to an embodiment, antibodies of the present invention, such as the h11B6 antibody, may be prepared as described in U.S. Pat. Nos. 10,100,125 and 9,873,891, both of which are hereby incorporated by reference. In some embodiments, an antibody of the present invention, such as h11B6, is prepared using CHO-DG44 cells.

Methods for the production of antibodies are well-known in the art. For example, suitable methods for the production of recombinant polypeptides are known in the art, such as expression in prokaryotic or eukaryotic hosts cells (for example, see Sambrook & Russell, 2000, Molecular Cloning, A Laboratory Manual, Third Edition, Cold Spring Harbor, N.Y., the relevant disclosures in which document are hereby incorporated by reference).

An aspect of the invention provides an isolated nucleic acid molecule encoding an antibody of the invention, or a component polypeptide chain thereof. A “nucleic acid molecule” includes DNA (e.g. genomic DNA or complementary DNA) and mRNA molecules, which may be single- or double-stranded. In one embodiment, the nucleic acid molecule is a cDNA molecule. It will be appreciated by persons skilled in the art that the nucleic acid molecule may be codon-optimised for expression of the antibody polypeptide in a particular host cell, e.g. for expression in human cells (for example, see Angov, 2011, Biotechnol. J. 6(6):650-659).

In a particular embodiment, the nucleic acid molecule of the invention comprises (a) the nucleotide sequence of SEQ ID NO: 14, and/or (b) the nucleotide sequence of SEQ ID NO: 15.

### Antibody Variants of the Present Invention

In some embodiments, amino acid sequence modification(s) of the antibodies provided herein are contemplated. For example, it may be desirable to improve the binding affinity and/or other biological properties of the antibody, including but not limited to specificity, thermostability, expression level, effector functions, glycosylation (e.g., fucosylation), reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites.

Accordingly, in one embodiment, an antibody that binds to an hK2 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for example, the amino acid sequence of an h11B6 antibody as described herein.

### Chelators of the Present Invention

According to particular embodiments, chelators of the present invention refer to a chelator to which a metal, preferably a radiometal, can be complexed to form a radiometal complex. Preferably, the chelator is a macrocyclic compound. In certain embodiments, a chelator comprises a macrocycle or a macrocyclic ring containing one or more heteroatoms, e.g., oxygen and/or nitrogen as ring atoms.

According to particular embodiments, the chelator comprises a macrocyclic chelating moiety. Examples of macrocyclic chelating moieties include, without limitation, 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof. In some aspects, the chelator is 1,4,7,10-tetraazacyclododecane-1,4,7,10,tetraacetic acid (DOTA). In other aspects, the chelator is S-2-(4-isothiocyanatobenzyl)-1,4,7-triazacyclononane-1,4,7-triacetic acid (NOTA). In further aspects, the chelator is 1,4,8,11-tetraazacyclodocedan-1,4,8,11-tetraacetic acid (TETA). In yet other aspects, the chelator is 3,6,9,15-tetraazabicyclo[9.3.1]-pentadeca-1(15),11,13-triene-4-(S)-(4-isothiocyanatobenzyl)-3,6,9-triacetic acid (PCTA). In still further aspects, the chelator is 5-S-(4-aminobenzyl)-1-oxa-4,7,10-triazacyclododecane-4,7,10-tris(acetic acid) (DO3A). In other aspects, the chelator is DOTA, DFO, DTPA, NOTA, or TETA.

In alternative embodiments, the chelator comprises a macrocycle that is a derivative of 4,13-diaza-18-crown-6. 4,13-Diaza-18-crown-6 may be prepared in a variety of ways (see, e.g., Gatto et al., Org. Synth. 1990, 68, 227; DOI: 10.15227/orgsyn.068.0227). According to further embodiments of the present invention, the chelator is H₂bp18c6 or a H₂bp18c6 derivative, such as those described in WO2020/229974. H₂bp18c6 refers to N,N′-bis[(6-carboxy-2-pyridil)methyl]-4,13-diaza-18-crown-6, as described herein. H₂bp18c6 and H₂bp18c6 derivatives are also described, for example, in Thiele et al. “An Eighteen-Membered Macrocyclic Ligand for Actinium-225 Targeted Alpha Therapy” Angew. Chem. Int. Ed. (2017) 56, 14712-14717, and Roca-Sabio et al. “Macrocyclic Receptor Exhibiting Unprecedented Selectivity for Light Lanthanides” J. Am. Chem. Soc. (2009) 131, 3331-3341, which are incorporated by reference herein. Additional chelators suitable for use in accordance with the present invention are described in WO2018/183906 and WO2020/106886, which are incorporated by reference herein.

As used herein, the term “TOPA” refers to a macrocycle known in the art as H₂bp18c6 and may alternatively be referred to as N,N′-bis[(6-carboxy-2-pyridil)methyl]-4,13-diaza-18-crown-6, or as 6,6′-((1,4,10,13-tetraoxa-7,16-diazacyclooctadecane-7,16-diyl)bis(methylene))dipicolinic acid. See, e.g., Roca-Sabio et al.

### CHELATORS OF FORMULAE (I), (II) and (III)

Additional chelators suitable for use in accordance with the present invention are described in WO2020/229974, which is incorporated by reference herein. According to particular embodiments, e.g., as described in WO2020/229974, the chelator has the structure of Formula (I):

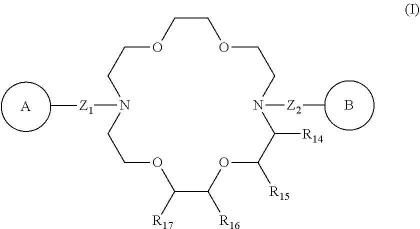

wherein:
each of ring A and ring B is independently a 6-10 membered aryl or a 5-10 membered heteroaryl, wherein each of ring A and ring B is optionally substituted with one or more substituents independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN—OC(O)N(R₁₃)₂, and X;
each of Z₁ and Z₂ is independently —(C(R₁₂)₂)ₘ— or —(CH₂)ₙ—C(R₁₂)(X)—(CH₂)ₙ—;
each X is independently -L₁-R₁₁;
each n is independently 0, 1, 2, 3, 4, or 5;
each m is independently 1, 2, 3, 4, or 5;
each p is independently 0 or 1;
L₁ is absent or a linker;
R₁₁ is a nucleophilic moiety or an electrophilic moiety, or R₁₁ comprises an antibody or antigen binding domain;
each R₁₂ is independently hydrogen, alkyl, cycloalkyl, aryl, heterocyclyl, or heteroaryl;
each R₁₃ is independently hydrogen or alkyl;
each of R₁₄, R₁₅, R₁₆, and R₁₇ is independently hydrogen, alkyl, or X, or alternatively R₁₄ and R₁₅ and/or R₁₆ and R₁₇ are taken together with the carbon atoms to which they are attached to form a 5- or 6-membered cycloalkyl ring optionally substituted with X;
provided that the chelator comprises at least one X, and when X is present on ring A or ring B, L₁ is a linker or at least one of R₁₂ and R₁₄-R₁₇ is not hydrogen.

According to embodiments of the invention, a chelator comprises at least one X group, wherein X is -L₁-R₁₁, wherein L₁ is absent or a linker, and R₁₁ is an electrophilic moiety or a nucleophilic moiety, or R₁₁ comprises an antibody or antigen binding domain. When R₁₁ is a nucleophilic or electrophilic moiety, such moiety can be used for attachment of the chelator to an antibody or antigen binding domain, directly or indirectly via a linker. According to preferred embodiments, R₁₁ comprises an antibody with binding specificity for hK2, such as h11B6.

In certain embodiments, a chelator comprises a single X group, and preferably L₁ of the X group is a linker.

A chelator of the invention can be substituted with X at any one of the carbon atoms of the macrocyclic ring, the Z₁ or Z₂ position, or on ring A or ring B, provided that when ring A or ring B comprises an X group, L₁ is a linker or at least one of R₁₂ and R₁₄-R₁₇ is not hydrogen (i.e., at least one of the carbon atoms of Z₁, Z₂, and/or the carbons of the macrocyclic ring is substituted for instance with an alkyl group, such as methyl or ethyl). Preferably, substitution at such positions does not affect the chelation efficiency of the chelator for radiometal ions, particularly ²²⁵Ac, and in some embodiments, the substitutions can enhance chelation efficiency.

In some embodiments, L₁ is absent. When L₁ is absent, R₁₁ is directly bound (e.g., via covalent linkage) to the chelator.

In some embodiments, L₁ is a linker. As used herein with respect to compounds of Formulae (I), (II), (III), (IV), (V) and (VI), L₁ refers to a chemical moiety that joins a chelator to a nucleophilic moiety, electrophilic moiety, antibody or antigen binding domain. Any suitable linker known to those skilled in the art in view of the present disclosure can be used in the invention. The linkers can contain, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB). Exemplary linker structures suitable for use include, but are not limited to:

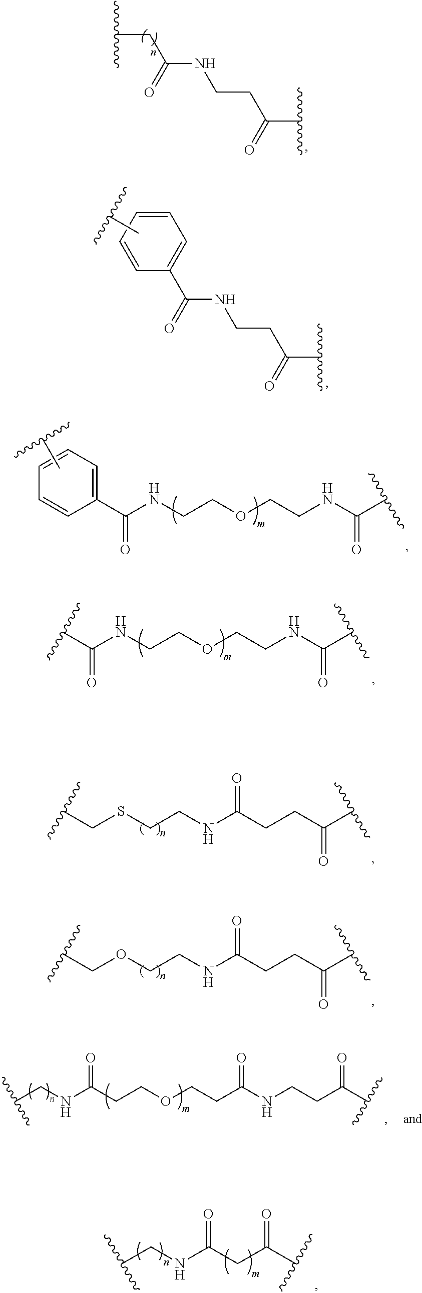

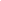

wherein n is an integer of 0 to 10, preferably an integer of 1 to 4; and m is an integer of 0 to 12, preferably an integer of 0 to 6.

In some embodiments, R₁₁ is a nucleophilic moiety or an electrophilic moiety. A “nucleophilic moiety” or “nucleophilic group” refers to a functional group that donates an electron pair to form a covalent bond in a chemical reaction. An “electrophilic moiety” or “electrophilic group” refers to a functional group that accepts an electron pair to form a covalent bond in a chemical reaction. Nucleophilic groups react with electrophilic groups, and vice versa, in chemical reactions to form new covalent bonds. Reaction of the nucleophilic group or electrophilic group of a chelator of the invention with an antibody or antigen binding domain or other chemical moiety (e.g., linker) comprising the corresponding reaction partner allows for covalent linkage of the antibody or antigen binding domain or chemical moiety to the chelator of the invention.

Exemplary examples of nucleophilic groups include, but are not limited to, azides, amines, and thiols. Exemplary examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups suitable for use in the invention include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups suitable for use in the invention include, but are not limited to, Michael acceptors (e.g., maleimide), haloacetyl, acyl halides, activated disulfides, and phenyloxadiazole sulfone.

In particular embodiments, R₁₁ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, acyl halide (e.g., —C(O)Cl, —C(O)Br), tetrazine, or trans-cyclooctene wherein each R₁₃ is independently hydrogen or alkyl.

In some embodiments, R₁₁ is an alkynyl, cycloalkynyl, or azido group thus allowing for attachment of the chelator to an antibody or antigen binding domain or other chemical moiety (e.g., linker) using a click chemistry reaction. In such embodiments, the click chemistry reaction that can be performed is a Huisgen cycloaddition or 1,3-dipolar cycloaddition between an azido (—N₃) and an alkynyl or cycloalkynyl group to form a 1,2,4-triazole linker or moiety. In one embodiment, the chelator comprises an alkynyl or cycloalkynyl group and the antibody or antigen binding domain or other chemical moiety comprises an azido group. In another embodiment, the chelator comprises an azido group and the antibody or antigen binding domain or other chemical moiety comprises an alkynyl or cycloalkynyl group.

In certain embodiments, R₁₁ is an alkynyl group, more preferably a terminal alkynyl group or cycloalkynyl group that is reactive with an azide group, particularly via strain-promoted azide-alkyne cycloaddition (SPAAC). Examples of cycloalkynyl groups that can react with azide groups via SPAAC include, but are not limited to cyclooctynyl or a bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFB0), monobenzocyclooctynyl (MOBO), and tetramethoxy dibenzocyclooctynyl (TMDIBO).

In a particular embodiment, R₁₁ is dibenzoazacyclooctynyl (DIBAC, DBCO, ADIBO), which has the following structure:

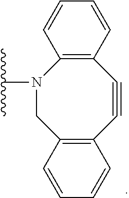 [SMILES: *N1Cc2ccccc2C#Cc2ccccc21]

In such embodiments in which R₁₁ is DBCO, the DBCO can be covalently linked to a chelator directly or indirectly via a linker, and is preferably attached to the chelator indirectly via a linker.

In some embodiments, R₁₁ comprises an antibody or antigen binding domain. The antibody or antigen binding domain can be linked to the chelator directly via a covalent linkage, or indirectly via a linker. According to preferred embodiments, R₁₁ comprises an antibody with binding specificity for hK2, such as h11B6.

According to embodiments of the invention, each of ring A and ring B is independently a 6-10 membered aryl or a 5-10 membered heteroaryl. In alternative embodiments, it is contemplated that each of ring A and ring B is an optionally substituted heterocyclyl ring, such as oxazoline. Each of ring A and ring B can be optionally and independently substituted with one or more substituent groups independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN—OC(O)N(R₁₃)₂, and X. Examples of 6-10 membered aryl groups suitable for this purpose include, but are not limited to, phenyl and naphthyl. Examples of 5 to 10 membered heteroaryl groups suitable for this purpose include, but are not limited to pyridinyl, thiazolyl, isothiazolyl, oxazolyl, isoxazolyl, and imidazolyl. Examples of suitable substituents of the 5 to 10 membered heteroaryl and 6 to 10 membered aryl groups include, but are not limited to —COOH, tetrazolyl, and —CH₂COOH. In preferred embodiments, a substituent group is —COOH or tetrazolyl, which is an isostere of —COOH.

In certain embodiments, each of ring A and ring B are independently and optionally substituted with one or more carboxyl groups, including but not limited to, —COOH and —CH₂COOH.

In certain embodiments, each of ring A and ring B are independently and optionally substituted with tetrazolyl.

In one embodiment, ring A and ring B are the same, e.g., both ring A and ring B are pyridinyl. In another embodiment, ring A and ring B are different, e.g., one of ring A and ring B is pyridinyl and the other is phenyl.

In a particular embodiment, both ring A and ring B are pyridinyl substituted with —COOH.

In a particular embodiment, both ring A and ring B are pyridinyl substituted with tetrazolyl.

In another particular embodiment, both ring A and ring B are picolinic acid groups having the following structure:

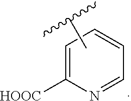

According to embodiments of the invention, each of Z₁ and Z₂ is independently —(C(R₁₂)₂)ₘ— or —(CH₂)ₙ—C(R₁₂)(X)—(CH₂)ₙ—; each X is independently -L₁-R₁₁; each R₁₂ is independently hydrogen, alkyl, cycloalkyl, aryl, heterocyclyl, or heteroaryl; each n is independently 0, 1, 2, 3, 4, or 5; and each m is independently 1, 2, 3, 4, or 5.

In some embodiments, each R₁₂ is independently hydrogen or alkyl, more preferably hydrogen, —CH₃, or —CH₂CH₃.

In some embodiments, each R₁₂ is hydrogen.

In some embodiments, both Z₁ and Z₂ are —(CH₂)ₘ—, wherein each m is preferably 1. In such embodiments, a carbon atom of the macrocyclic ring, ring A, or ring B is substituted with an X group.

In some embodiments, one of Z₁ and Z₂ is —(CH₂)ₙ—C(R₁₂)(X)—(CH₂)ₙ— and the other is —(CH₂)ₘ—.

In some embodiments, one of Z₁ and Z₂—(CH₂)ₙ—C(R₁₂)(X)—(CH₂)ₙ— and the other is —(CH₂)ₘ—; each n is 0; m is 1; X is -L₁-R₁₁; and L₁ is a linker.

In some embodiments, both Z₁ and Z₂ are —(CH₂)ₘ—; each m is independently 0, 1, 2, 3, 4, or 5, preferably each m is 1; and one of R₁₄, R₁₅, R₁₆, and R₁₇ is X, and the rest of R₁₄, R₁₅, R₁₆, and R₁₇ are each hydrogen.

In some embodiments, R₁₄ and R₁₅ are taken together with the carbon atoms to which they are attached to form a 5- or 6-membered cycloalkyl ring (i.e., cyclopentyl or cyclohexyl). Such 5- or 6-membered cycloalkyl rings can be substituted with an X group.

In some embodiments R₁₆ and R₁₇ are taken together with the carbon atoms to which they are attached to form a 5- or 6-membered cycloalkyl ring (i.e., cyclopentyl or cyclohexyl). Such 5- or 6-membered cycloalkyl rings can be substituted with an X group.

In certain embodiments, a chelator has the structure of Formula (II):

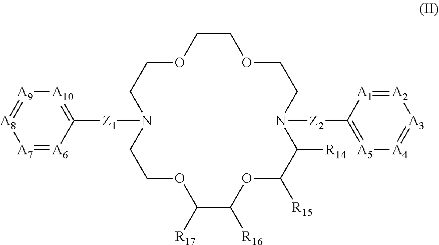

wherein:
   A₁ is N or CR₁ or is absent;
   A₂ is N or CR₂;
   A₃ is N or CR₃;
   A₄ is N or CR₄;
   A₅ is N or CR₅;
   A₆ is N or CR₆ or is absent;
   A₇ is N or CR₇;
   A₈ is N or CR₈;
   A₉ is N or CR₉;
   A₁₀ is N or CR₁₀;
provided that no more than three of A₁, A₂, A₃, A₄, and A₅ are N, and no more than three of A₆, A₇, A₈, A₉, and A₁₀ are N;
   each of R₁, R₂, R₃, R₄, R₅, R₆, R₇, R₈, R₉, and R₁₀ is independently selected from the group consisting of hydrogen, halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN, —OC(O)N(R₁₃)₂, and —X,
   or, alternatively, any two directly adjacent R₁, R₂, R₃, R₄, R₅, R₆, R₇, R₈, R₉, and R₁₀ are taken together with the atoms to which they are attached to form a five or six-membered substituted or unsubstituted carbocyclic or nitrogen-containing ring;
   and Z₁, Z₂, X, n, m, p, L₁, and R₁₁-R₁₇ are as described above for formula (I),
   provided that the chelator comprises at least one X, and when any one of R₁, R₂, R₃, R₄, R₅, R₆, R₇, R₈, R₉, and R₁₀ is X, then L₁ is a linker or at least one of R₁₂ and R₁₄-R₁₇ is not hydrogen.

In some embodiments, any two directly adjacent R₁, R₂, R₃, R₄, R₅, R₆, R₇, R₈, R₉, and R₁₀ are taken together with the atoms to which they are attached to form a five or six-membered substituted or unsubstituted carbocyclic or nitrogen-containing ring. Examples of such carbocyclic rings that can be formed include, but are not limited to, naphthyl. Examples of such nitrogen-containing rings that can be formed include, but are not limited to, quinolinyl. The carbocyclic or nitrogen-containing rings can be unsubstituted or substituted with one or more suitable substituents, e.g., —COOH, —CH₂COOH, tetrazolyl etc.

In some embodiments, L₁ is absent. When L₁ is absent, R₁₁ is directly bound (e.g., via covalent linkage) to the chelator.

In some embodiments, L₁ is a linker. Any suitable linker known to those skilled in the art in view of the present disclosure can be used in the invention, such as those described above.

In some embodiments, one of A₁, A₂, A₃, A₄, and A₅ is nitrogen, one of A₁, A₂, A₃, A₄, and A₅ is carbon substituted with —COOH and the rest are CH, i.e., forming a pyridinyl ring substituted with carboxylic acid.

In some embodiments, one of A₆, A₇, A₈, A₉, and A₁₀ is nitrogen, one of A₆, A₇, A₈, A₉, and A₁₀ is carbon substituted with —COOH, and the rest are CH, i.e., forming a pyridinyl ring substituted with carboxylic acid.

In one embodiment, at least one of R₁, R₂, R₃, R₄ and R₅ is —COOH. In one embodiment, at least one of R₆, R₇, R₈, R₉, and R₁₀ is —COOH. In another embodiment, at least one of R₁, R₂, R₃, R₄ and R₅ is —COOH; and at least one of R₆, R₇, R₈, R₉, and R₁₀ is —COOH.

In some embodiments, each of A₁ and A₁₀ is nitrogen; A₂ is CR₂ and R₂ is —COOH; A₉ is CR₉ and R₉ is —COOH; each of A₃-A₈ is CR₂, CR₃, CR₄, CR₅, CR₆, CR₇, and CR₈, respectively; and each of R₃ to R₈ is hydrogen.

In some embodiments, one of A₁, A₂, A₃, A₄, and A₅ is nitrogen, one of A₁, A₂, A₃, A₄, and A₅ is carbon substituted with tetrazolyl and the rest are CH.

In some embodiments, one of A₆, A₇, As, A₉, and A₁₀ is nitrogen, one of A₆, A₇, A₈, A₉, and A₁₀ is carbon substituted with tetrazolyl, and the rest are CH.

In one embodiment, at least one of R₁, R₂, R₃, R₄ and R₅ is tetrazolyl. In one embodiment, at least one of R₆, R₇, R₈, R₉, and R₁₀ is tetrazolyl. In another embodiment, at least one of R₁, R₂, R₃, R₄ and R₅ is tetrazolyl; and at least one of R₆, R₇, R₈, R₉, and R₁₀ is tetrazolyl.

In some embodiments, each R₁₂ is hydrogen.

In some embodiments, R₁₁ is an alkynyl group or cycloalkynyl group, preferably cyclooctynyl or a cyclooctynyl derivative, e.g., DBCO.

In particular embodiments of a chelator of formula (II):
each of A₁ and A₁₀ is nitrogen;

A₂ is CR₂ and R₂ is —COOH;

A₉ is CR₉ and R₉ is —COOH;

each of A₃-A₈ is CR₂, CR₃, CR₄, CR₅, CR₆, CR₇, and CR₈, respectively;

each of R₃ to R₈ is hydrogen;

one of Z₁ and Z₂ is —(CH₂)ₘ— and the other of Z₁ and Z₂ is —(CH₂)ₙ—C(R₁₂)(X)—(CH₂)ₙ—;

R₁₂ is hydrogen;

m is 1;

each n is 0;

X is -L₁-R₁₁, wherein L₁ is a linker and —R₁₁ is an electrophilic group, e.g., cyclooctynyl or cyclooctynyl derivative such as DBCO; and

each of R₁₄-R₁₇ is hydrogen, or alternatively R₁₆ and R₁₇ are taken together with the carbon atoms to which they are attached to form a 5- or 6-membered cycloalkyl ring.

In certain embodiments, a chelator has the structure of formula (III):

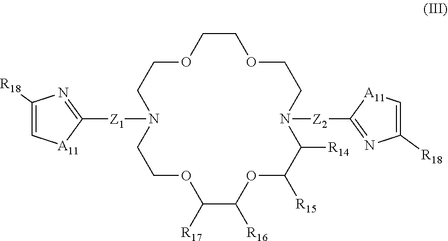

wherein:
each A₁₁ is independently O, S, NMe, or NH;
each R₁₈ is independently selected from the group consisting of hydrogen, halo, alkyl, alkenyl, cycloalkyl, cycloalkenyl, aryl, heterocyclyl, heteroaryl, —OR₁₃, —SR₁₃, —COOR₁₃, —OC(O)R₁₃, —N(R₁₃)₂, —CON(R₁₃)₂, —NO₂, —CN—OC(O)N(R₁₃)₂, and —X,
and Z₁, Z₂, X, n, m, L₁₁, R₁₁-R₁₇ are as described above for formula (I),
provided that the chelator comprises at least one X, and when R₁₈ is X, then L₁ is a linker or at least one of R₁₂ and R₁₄-R₁₇ is not hydrogen.

In some embodiments, each A₁₁ is the same, and each A₁₁ is O, S, NMe, or NH. For example, each A₁₁ can be S. In other embodiments, each A₁₁ is different and each is independently selected from O, S, NMe, and NH.

In some embodiments, each R₁₈ is independently —(CH₂)ₚ—COOR₁₃ or tetrazolyl, wherein R₁₃ is hydrogen and each p is independently 0 or 1.

In some embodiments, each R₁₈ is —COOH.

In some embodiments, each R₁₈ is —CH₂COOH.

In some embodiments, each R₁₈ is tetrazolyl.

In particular embodiments of a chelator of formula (III):
each R₁₈ is COOH;
one of Z₁ and Z₂ is —(CH₂)ₘ— and the other of Z₁ and Z₂ is —(CH₂)ₙ—C(R¹²)(X)—(CH₂)ₙ—;
R₁₂ is hydrogen;
m is 1; each n is 0;
X is -L₁-R₁₁, wherein L₁ is a linker and —R₁₁ is an electrophilic group, e.g., cyclooctynyl or cyclooctynyl derivative such as DBCO, or BCN; and
each of R₁₄-R₁₇ is hydrogen, or alternatively R₁₆ and R₁₇ are taken together with the carbon atoms to which they are attached to form a 5- or 6-membered cycloalkyl ring.

In particular embodiments of the invention, a chelator is selected from the group consisting of:

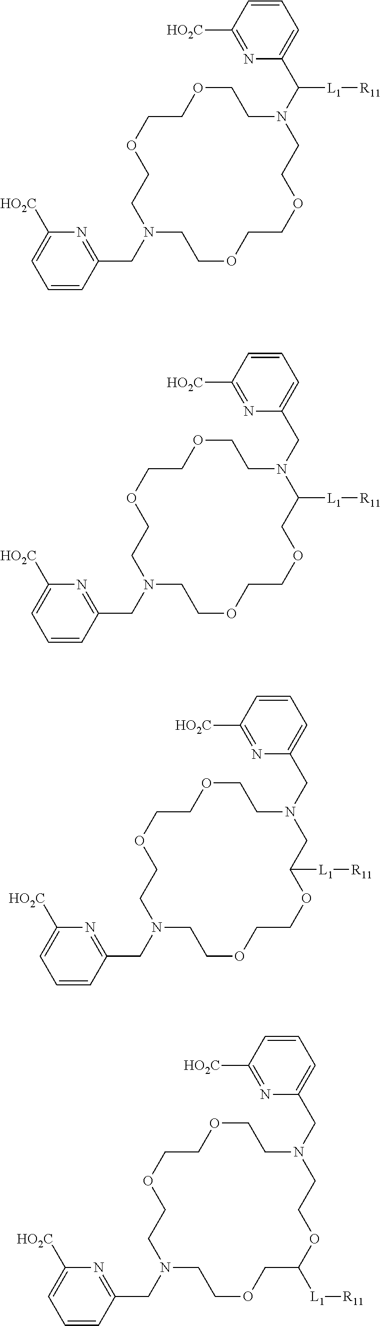

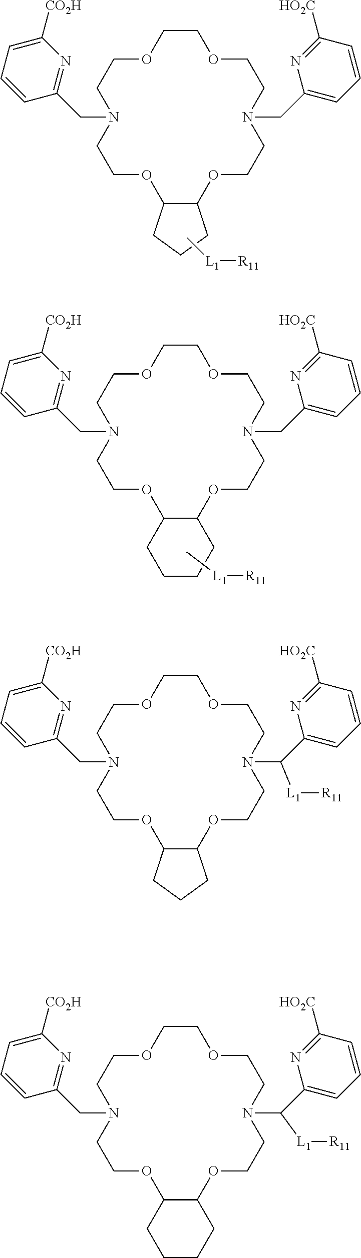

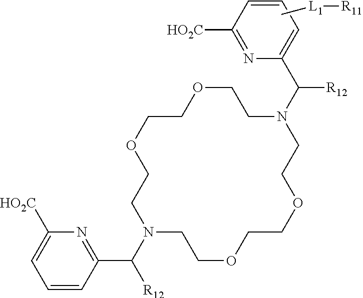

wherein:
L₁ is absent or a linker;
R₁₁ is a nucleophilic moiety or an electrophilic moiety, or R₁₁ comprises an antibody or antigen binding domain (e.g., h11B6); and
each R₁₂ is independently hydrogen, —CH₃, or —CH₂CH₃, provided at least one R₁₂ is —CH₃ or —CH₂CH₃.

In some embodiments, R₁₁ is —NH₂, —NCS, —NCO, —N₃, alkynyl, cycloalkynyl, —C(O)R₁₃, —COOR₁₃, —CON(R₁₃)₂, maleimido, acyl halide, tetrazine, or trans-cyclooctene.

In certain embodiments, R₁₁ is cyclooctynyl or a cyclooctynyl derivative selected from the group consisting of bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

Preferably, R₁₁ is an alkynyl group or cycloalkynyl group, more preferably a cycloalkynyl group, e.g., DBCO or BCN.

Exemplary chelators of the invention include, but are not limited to:

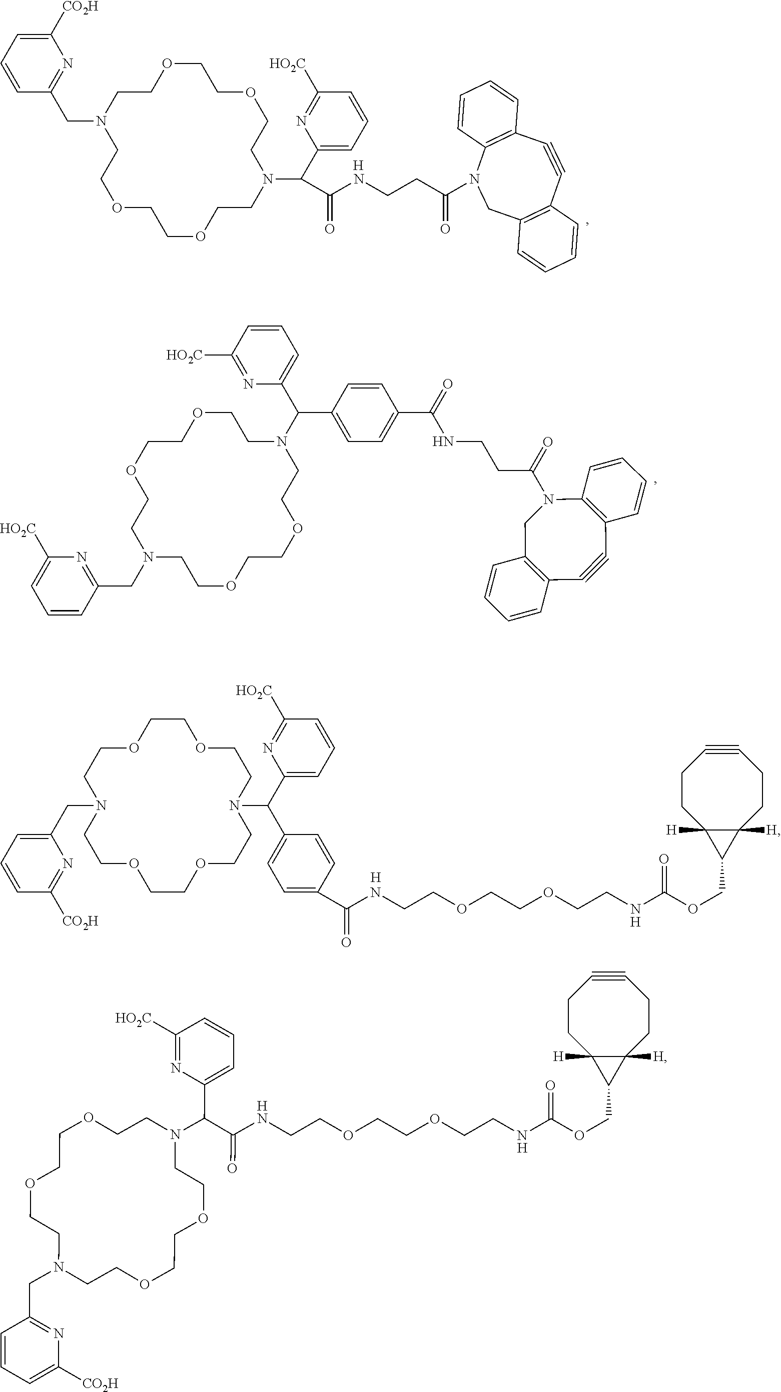

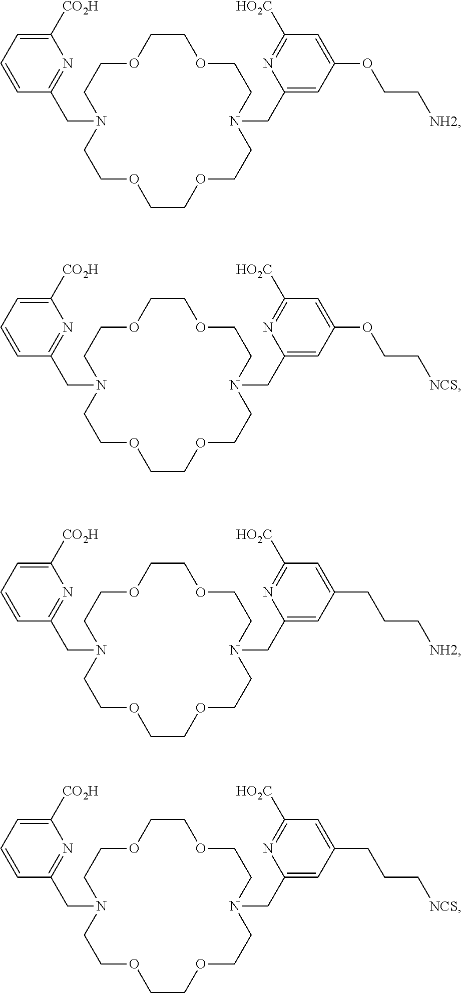

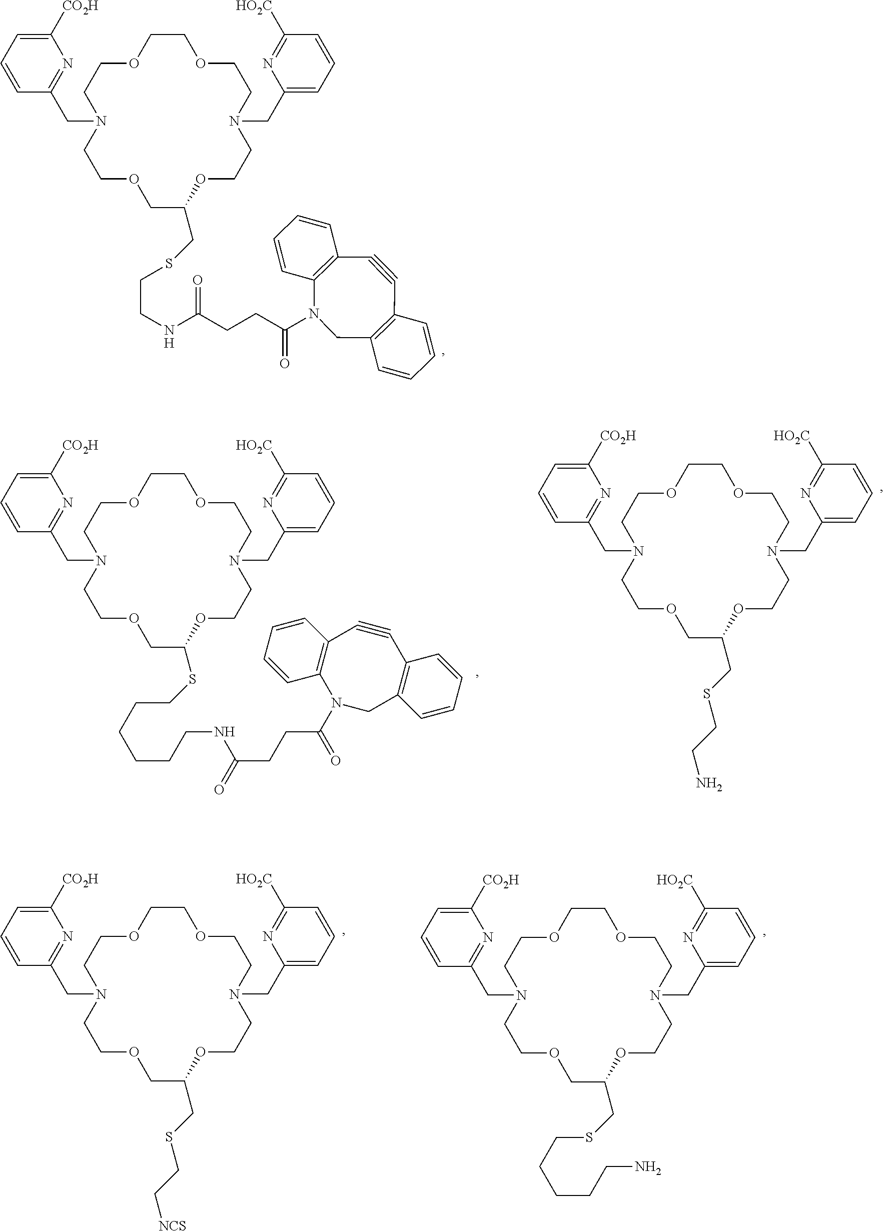

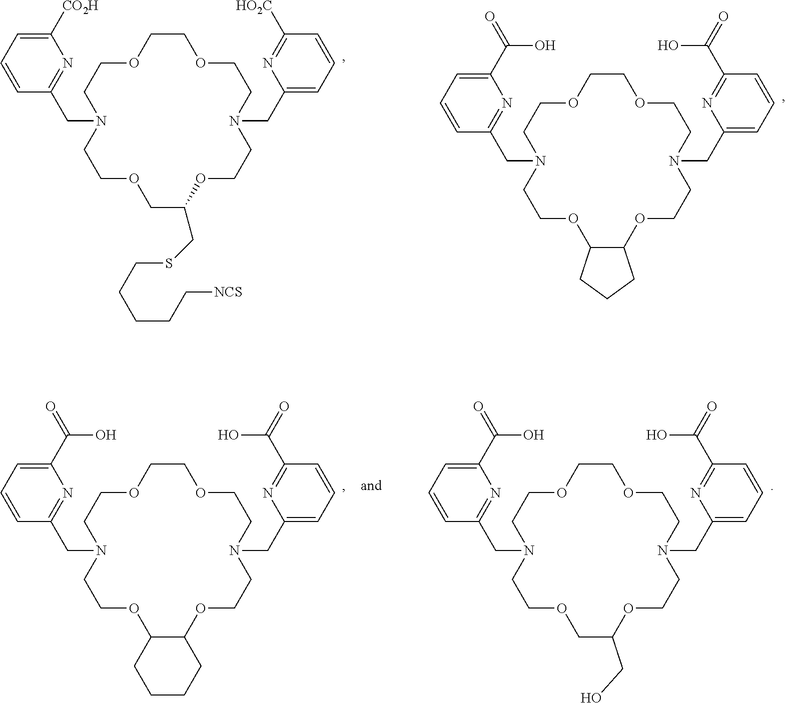

Such chelators can be covalently attached to an antibody or antigen binding domain to form immunoconjugates or radioimmunoconjugates by reacting the chelator with an azide-labeled antibody or antigen binding domain to form a 1,2,3-triazole linker via a click chemistry reaction as described in WO 2020/229974.

Chelators of the invention can be produced by any method known in the art in view of the present disclosure. For example, the pendant aromatic/heteroaromatic groups can be attached to the macrocyclic ring portion by methods known in the art, such as those exemplified and described in WO 2020/229974.

### Chelators of Formulae (IV), (V) and (VI)

Additional chelators suitable for use in accordance with the present invention are described in PCT/IB2021/060350, which is incorporated by reference herein. According to particular embodiments, e.g., as described in PCT/IB2021/060350, the chelator has the structure of formula (IV),

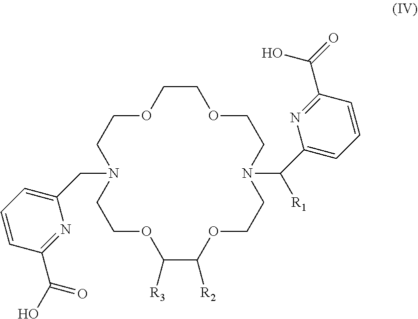

or a pharmaceutically acceptable salt thereof, wherein:
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker; and
R₄ is a nucleophilic moiety, an electrophilic moiety, or an antibody or antigen binding domain (e.g., h11B6).

In some embodiments, L₁ is absent. When L₁ is absent, R₄ is directly bound (e.g., via covalent linkage) to the compound.

In some embodiments, L₁ is a linker. As used herein, the term “linker” refers to a chemical moiety that joins a compound of the invention to a nucleophilic moiety, electrophilic moiety, or an antibody or antigen binding domain. Any suitable linker known to those skilled in the art in view of the present disclosure can be used in the invention. The linkers can have, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB). Exemplary linker structures suitable for use in the invention include, but are not limited to:

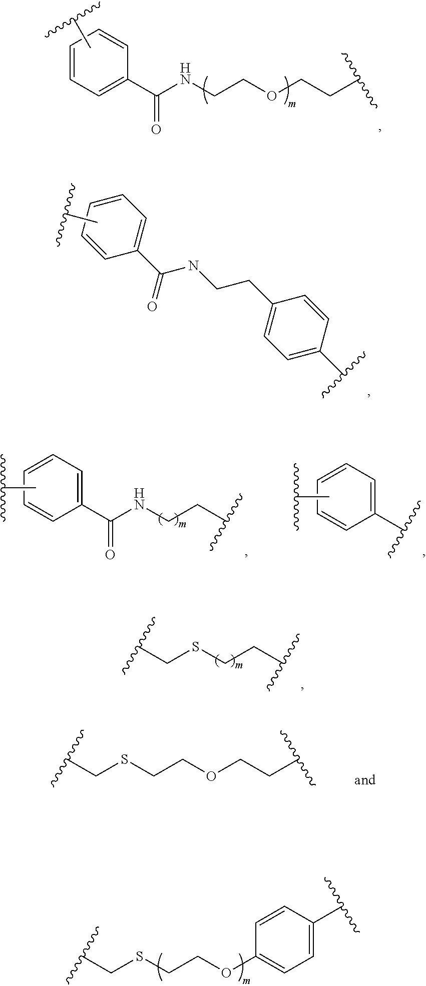

wherein m is an integer of 0 to 12.

In some embodiments, R₄ is a nucleophilic moiety or an electrophilic moiety. A “nucleophilic moiety” or “nucleophilic group” refers to a functional group that donates an electron pair to form a covalent bond in a chemical reaction. An “electrophilic moiety” or “electrophilic group” refers to a functional group that accepts an electron pair to form a covalent bond in a chemical reaction. Nucleophilic groups react with electrophilic groups, and vice versa, in chemical reactions to form new covalent bonds. Reaction of the nucleophilic group or electrophilic group of a compound of the invention with an antibody or antigen binding domain or other chemical moiety (e.g., linker) comprising the corresponding reaction partner allows for covalent linkage of the antibody or antigen binding domain or chemical moiety to the compound of the invention.

Examples of nucleophilic groups include, but are not limited to, azides, amines, and thiols. Examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups suitable for use in the invention include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups suitable for use in the invention include, but are not limited to, Michael acceptors (e.g., maleimide), haloacetyl, acyl halides, activated disulfides, and phenyloxadiazole sulfone.

In certain embodiments, R₄ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, acyl halide (e.g., —C(O)Cl, —C(O)Br), tetrazine, or trans-cyclooctene wherein each R₁₃ is independently hydrogen or alkyl.

In some embodiments, R₄ is an alkynyl, cycloalkynyl, or azido group thus allowing for attachment of the compound of the invention to an antibody or antigen binding domain or other chemical moiety (e.g., linker) using a click chemistry reaction. In such embodiments, the click chemistry reaction that can be performed is a Huisgen cycloaddition or 1,3-dipolar cycloaddition between an azido (—N₃) and an alkynyl or cycloalkynyl group to form a 1,2,4-triazole linker or moiety. In one embodiment, the compound of the invention comprises an alkynyl or cycloalkynyl group and the antibody or antigen binding domain or other chemical moiety comprises an azido group. In another embodiment, the compound of the invention comprises an azido group and the antibody or antigen binding domain or other chemical moiety comprises an alkynyl or cycloalkynyl group.

In certain embodiments, R₄ is an alkynyl group, more preferably a terminal alkynyl group or cycloalkynyl group that is reactive with an azide group, particularly via strain-promoted azide-alkyne cycloaddition (SPAAC). Examples of cycloalkynyl groups that can react with azide groups via SPAAC include, but are not limited to cyclooctynyl or a bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In certain embodiments, R₄ is dibenzoazacyclooctynyl (DIBAC, DBCO, ADIBO), which has the following structure:

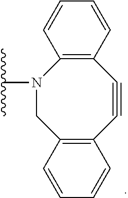 [SMILES: *N1Cc2ccccc2C#Cc2ccccc21]

In embodiments in which R₄ is DBCO, the DBCO can be covalently linked to a compound directly or indirectly via a linker, and is preferably attached to the compound indirectly via a linker.

In certain embodiments, R₄ comprises an antibody or antigen binding domain. The antibody or antigen binding domain can be linked to the compound directly via a covalent linkage, or indirectly via a linker. In preferred embodiments, the antibody or antigen binding domain has binding specificity for hK2, such as h11B6.

In another embodiment, the chelator is directed to a compound of formula (V):

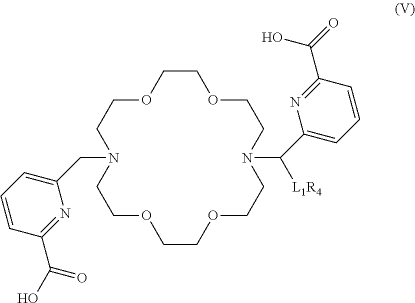

or a pharmaceutically acceptable salt thereof, wherein:
L₁ is absent or a linker; and
R₄ is a nucleophilic moiety, an electrophilic moiety, or an antibody or antigen binding domain.

In another embodiment the chelator is a compound of formula (VI):

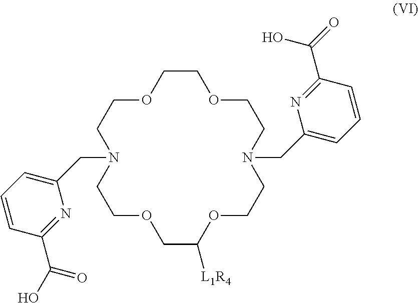

or a pharmaceutically acceptable salt thereof, wherein:
L₁ is absent or a linker; and
R₄ is a nucleophilic moiety, an electrophilic moiety, or an antibody or antigen binding domain (e.g., h11B6).

In another embodiment, the chelator is a compound as described above, wherein: R₁ is -L₁-R₄; R₂ and R₃ are taken together with the carbon atoms to which they are attached to form a 5- or 6-membered cycloalkyl; L₁ is absent or a linker; and R₄ is a nucleophilic moiety, an electrophilic moiety, or an antibody or antigen binding domain; or a pharmaceutically acceptable salt thereof.

In a further embodiment, the chelator is a compound as described above, wherein R₁ is H; R₂ and R₃ are taken together with the carbon atoms to which they are attached to form a 5- or 6-membered cycloalkyl substituted with -L₁-R₄; L₁ is absent or a linker; and R₄ is a nucleophilic moiety, an electrophilic moiety, or an antibody or antigen binding domain; or a pharmaceutically acceptable salt thereof:

Additional embodiments of the chelators described above include those wherein R₄ is an antibody. According to preferred embodiments, R₄ comprises an antibody with binding specificity for hK2, such as h11B6.

In an embodiment, the chelators are any one or more independently selected from the group consisting of the following compounds and pharmaceutically acceptable salts thereof:

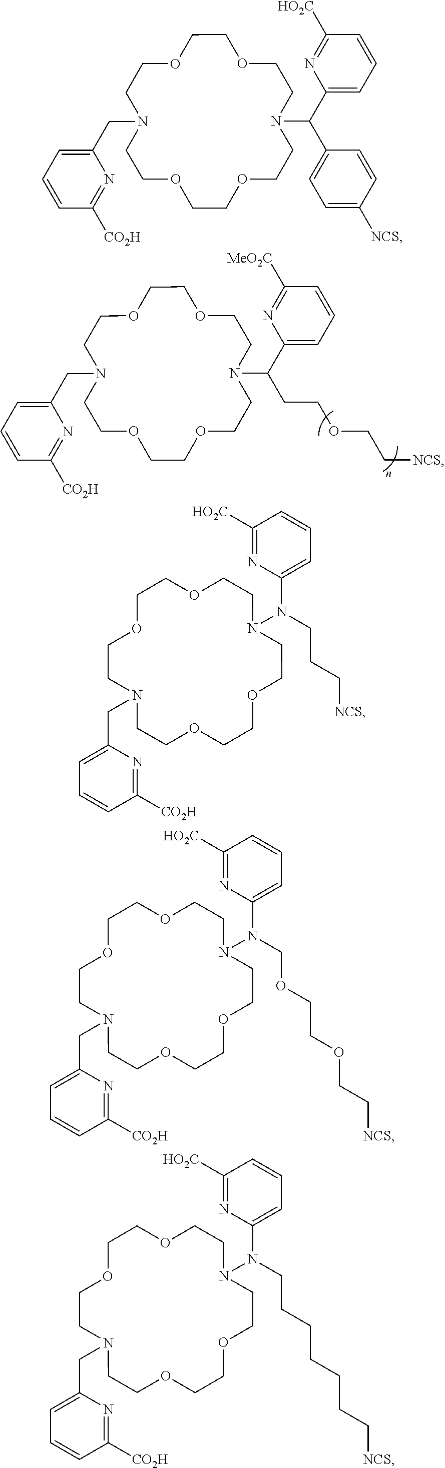

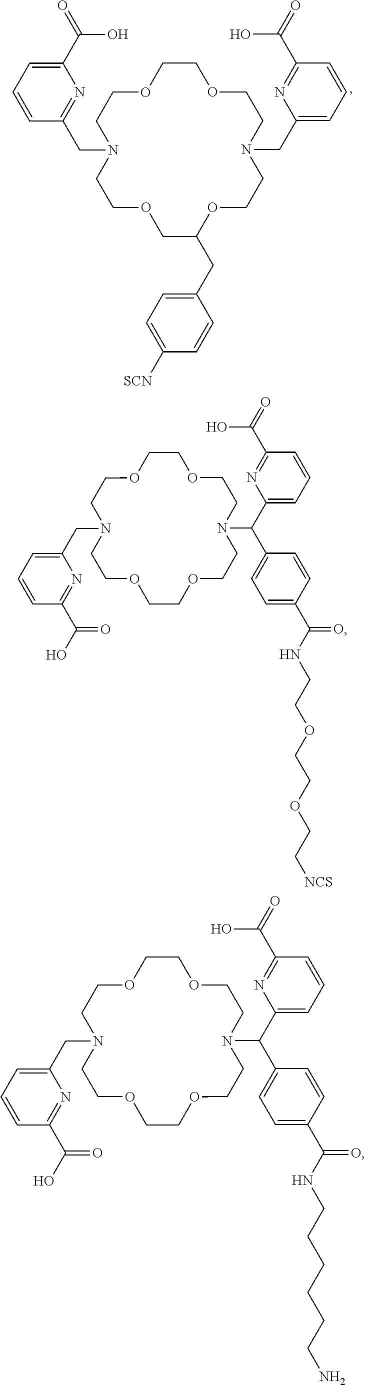

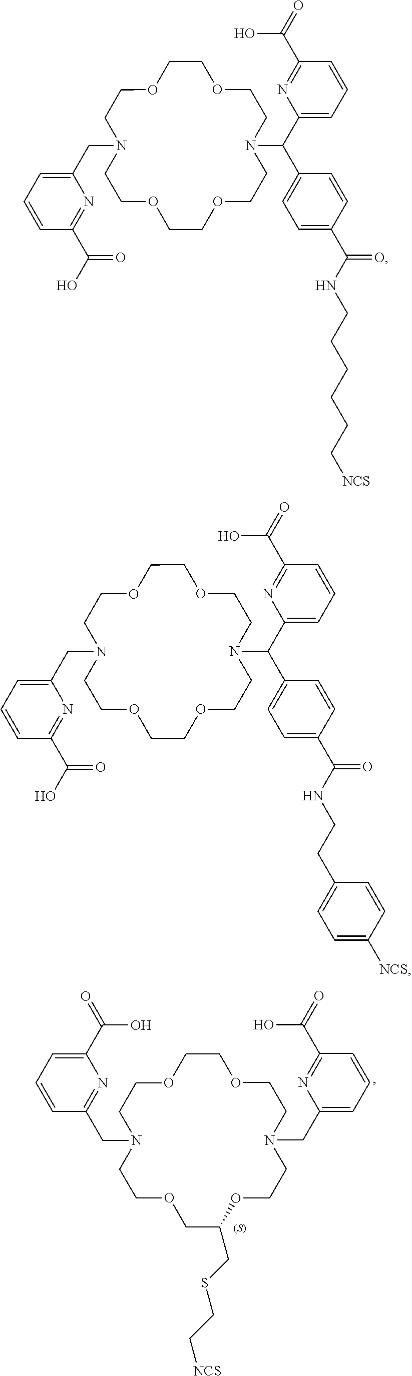

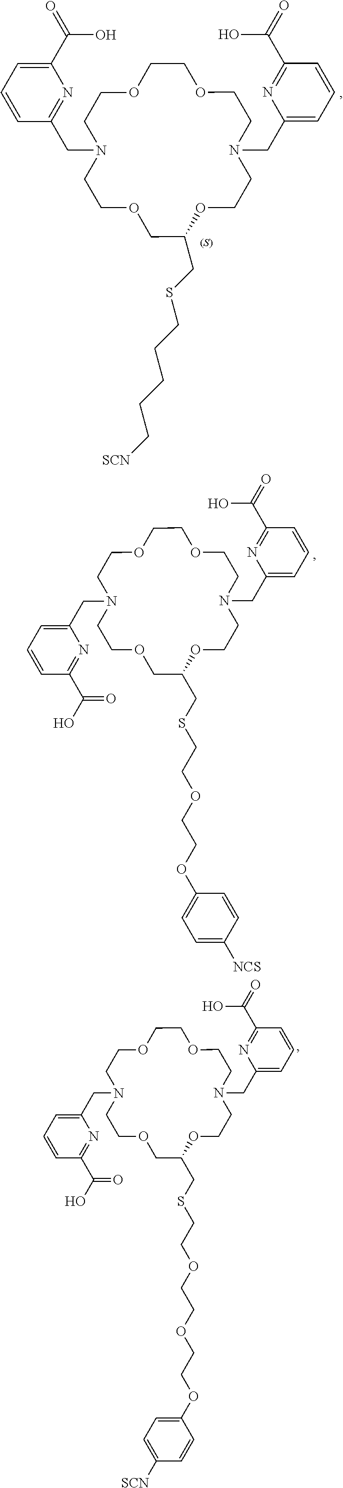

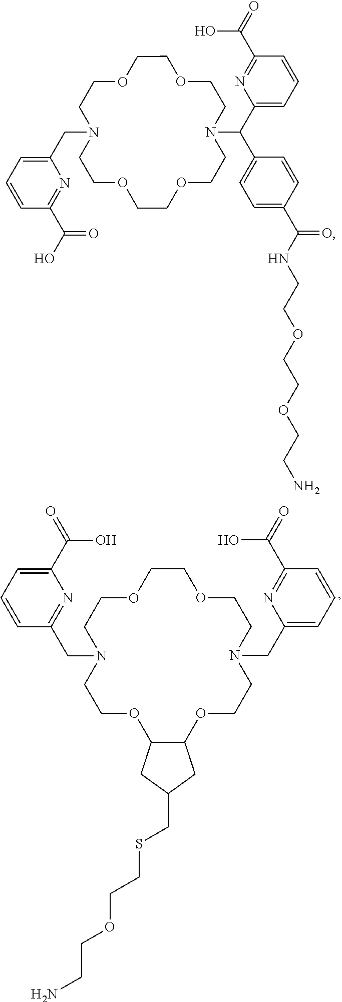

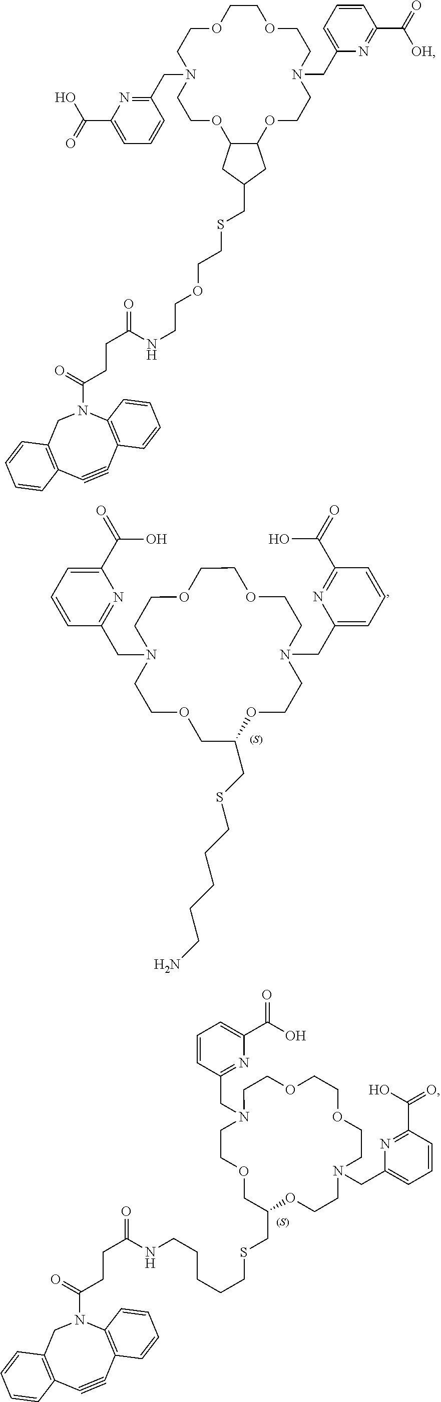

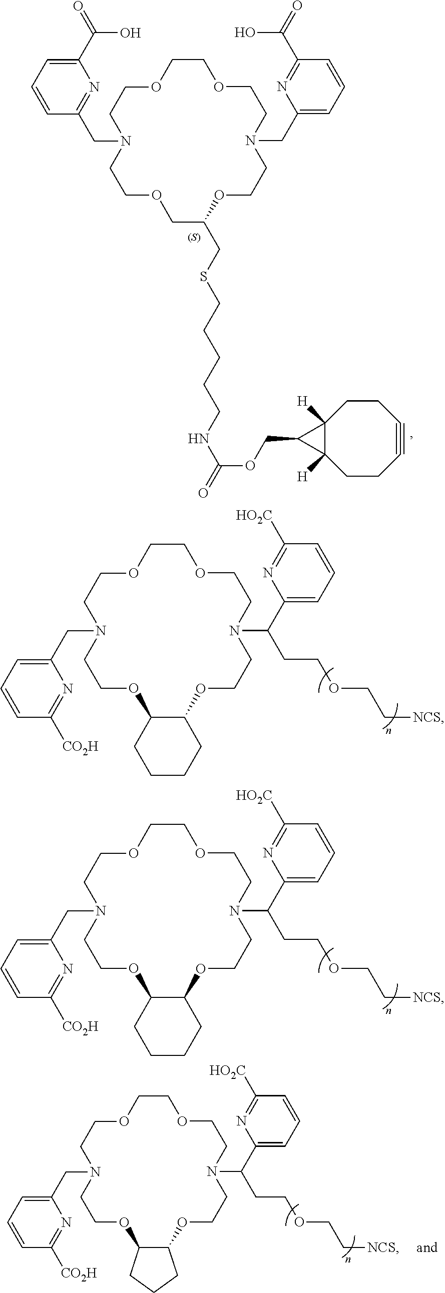

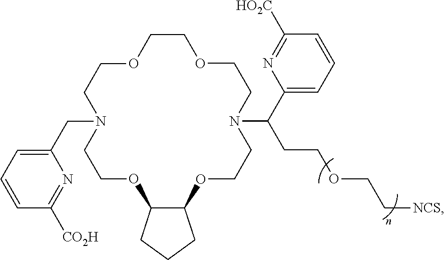

wherein n is 1-10.

In an embodiment, a radioconjugate of the present invention comprises a chelator of the following formula or a pharmaceutically acceptable salt thereof:

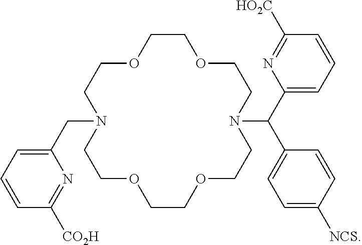

Said chelators can be covalently attached to an antibody or antigen binding domain (e.g., h11B6) to form immunoconjugates or radioimmunoconjugates by reacting the compound with an azide-labeled antibody or antigen binding domain to form a 1,2,3-triazole linker, e.g., via a click chemistry reaction as described in WO2020/229974, or as described in PCT/IB2021/060350.

Chelators, radiometal complexes and radioimmunoconjugates of the present invention can be produced by any method known in the art in view of the present disclosure; for example, the pendant aromatic/heteroaromatic groups can be attached to the macrocyclic ring portion by methods known in the art, such as those exemplified and described in WO2020/229974 and PCT/IB2021/060350.

### Chemical Nomenclature

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC).

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

The term “substituted” means that at least one hydrogen atom is replaced with a non-hydrogen group, provided that all normal valencies are maintained and that the substitution results in a stable compound. When a particular group is “substituted,” that group can have one or more substituents, preferably from one to five substituents, more preferably from one to three substituents, most preferably from one to two substituents, independently selected from the list of substituents. For example, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like. The term “independently” when used in reference to substituents, means that when more than one of such substituents is possible, such substituents can be the same or different from each other.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cm-Cn, such as C₁-C₁₁, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms; e.g., an alkyl group may contain 1 to 12 carbon atoms (C₁₋₁₂alkyl), or 1 to 8 carbon atoms (C₁₋₈alkyl), or 1 to 6 carbon atoms (C₁₋₆alkyl). Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, an alkenyl can have one carbon-carbon double bond, or multiple carbon-carbon double bonds, such as 2, 3, 4 or more carbon-carbon double bonds. Examples of alkenyl groups include, but are not limited to methenyl, ethenyl, propenyl, butenyl, etc. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl group can be a mono- or polycyclic alkyl group having from 3 to 12, more preferably from 3 to 8 carbon atoms in the ring(s) and comprising at least one double bond between two carbon atoms. Cycloalkenyl groups may be substituted or unsubstituted. In some embodiments the cycloalkenyl group may have one, two or three double bonds or multiple carbon-carbon double bonds, such as 2, 3, 4, or more carbon-carbon double bonds but does not include aromatic compounds. Cycloalkenyl groups have from 3 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C═CH, —C═CCH₃, —CH₂C═CCH₃, —C═CCH₂CH(CH₂CH₃)₂, among others. Alkynyl groups may be substituted or unsubstituted. A terminal alkyne has at least one hydrogen atom bonded to a triply bonded carbon atom. Representative substituted alkynyl groups may be mono-substituted or substituted more than once, such as, but not limited to, mono-, di- or trisubstituted with substituents such as those listed above. A “cyclic alkyne” or “cycloalkynyl” is a cycloalkyl ring comprising at least one triple bond between two carbon atoms. Examples of cyclic alkynes or cycloalkynyl groups include, but are not limited to, cyclooctyne, bicyclononyne (BCN), difluorinated cyclooctyne (DIFO), dibenzocyclooctyne (DIBO), keto-DIBO, biarylazacyclooctynone (BARAC), dibenzoazacyclooctyne (DIBAC), dimethoxyazacyclooctyne (DIMAC), difluorobenzocyclooctyne (DIFBO), monobenzocyclooctyne (MOBO), and tetramethoxy DIBO (TMDIBO).

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be monosubstituted or substituted more than once. For example, monosubstituted aryl groups include, but are not limited to, 2-, 3-, 4-, 5-, or 6-substituted phenyl or naphthyl groups, which may be substituted with substituents such as those listed above. Aryl moieties are well known and described, for example, in Lewis, R. J., ed., Hawley's Condensed Chemical Dictionary, 13^{th} Edition, John Wiley & Sons, Inc., New York (1997). An aryl group can be a single ring structure (i.e., monocyclic) or comprise multiple ring structures (i.e., polycyclic) that are fused ring structures. Preferably, an aryl group is a monocyclic aryl group.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

Similarly, alkylthio or thioalkoxy refers to an —SR group in which R is an alkyl attached to the parent molecule through a sulfur bridge, for example, —S-methyl, —S-ethyl, etc. Representative examples of alkylthio include, but are not limited to, —SCH₃, —SCH₂CH₃, etc.

The term “halogen” as used herein refers to bromine, chlorine, fluorine, or iodine. Correspondingly, the term “halo” means fluoro, chloro, bromo, or iodo. In some embodiments, the halogen is fluorine. In other embodiments, the halogen is chlorine or bromine.

The terms “hydroxy” and “hydroxyl” can be used interchangeably and refer to —OH.

The term “carboxy” refers to —COOH.

The term “cyano” refers to —CN.

The term “nitro” refers to —NO₂.

The term “isothiocyanate” refers to —N═C═S.

The term “isocyanate” refers to —N═C═O.

The term “azido” refers to —N₃.

The term “amino” refers to —NH₂. The term “alkylamino” refers to an amino group in which one or both of the hydrogen atoms attached to nitrogen is substituted with an alkyl group. An alkylamine group can be represented as —NR₂ in which each R is independently a hydrogen or alkyl group. For example, alkylamine includes methylamine (—NHCH₃), dimethylamine (—N(CH₃)₂), —NHCH₂CH₃, etc. The term “aminoalkyl” as used herein is intended to include both branched and straight-chain saturated aliphatic hydrocarbon groups substituted with one or more amino groups. Representative examples of aminoalkyl groups include, but are not limited to, —CH₂NH₂, —CH₂CH₂NH₂, and —CH₂CH(NH₂)CH₃.

As used herein, “amide” refers to —C(O)N(R)₂, wherein each R is independently an alkyl group or a hydrogen. Examples of amides include, but are not limited to, —C(O)NH₂, —C(O)NHCH₃, and —C(O)N(CH₃)₂.

The terms “hydroxylalkyl” and “hydroxyalkyl” are used interchangeably, and refer to an alkyl group substituted with one or more hydroxyl groups. The alkyl can be a branched or straight-chain aliphatic hydrocarbon. Examples of hydroxylalkyl include, but are not limited to, hydroxylmethyl (—CH₂OH), hydroxylethyl (—CH₂CH2OH), etc.

As used herein, the term “heterocyclyl” includes stable monocyclic and polycyclic hydrocarbons that contain at least one heteroatom ring member, such as sulfur, oxygen, or nitrogen. As used herein, the term “heteroaryl” includes stable monocyclic and polycyclic aromatic hydrocarbons that contain at least one heteroatom ring member such as sulfur, oxygen, or nitrogen. Heteroaryl can be monocyclic or polycyclic, e.g., bicyclic or tricyclic. Each ring of a heterocyclyl or heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring but the other fused ring or rings can be aromatic or non-aromatic. The heterocyclyl or heteroaryl group can be attached at any available nitrogen or carbon atom of any ring of the heterocyclyl or heteroaryl group. Preferably, the term “heteroaryl” refers to 5- or 6-membered monocyclic groups and 9- or 10-membered bicyclic groups which have at least one heteroatom (O, S, or N) in at least one of the rings, wherein the heteroatom-containing ring preferably has 1, 2, or 3 heteroatoms, more preferably 1 or 2 heteroatoms, selected from O, S, and/or N. The nitrogen heteroatom(s) of a heteroaryl can be substituted or unsubstituted. Additionally, the nitrogen and sulfur heteroatom(s) of a heteroaryl can optionally be oxidized (i.e., N→O and S(O)ᵣ, wherein r is 0, 1 or 2).

The term “ester” refers to —C(O)₂R, wherein R is alkyl.

The term “carbamate” refers to —OC(O)NR₂, wherein each R is independently alkyl or hydrogen.

The term “aldehyde” refers to —C(O)H.

The term “carbonate” refers to —OC(O)OR, wherein R is alkyl.

The term “maleimide” refers to a group with the chemical formula H₂C₂(CO)₂NH. The term “maleimido” refers to a maleimide group covalently linked to another group or molecule. Preferably, a maleimido group is N-linked, for example:

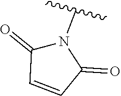 [SMILES: *N1C(=O)C=CC1=O]

The term “acyl halide” refers to —C(O)X, wherein X is halo (e.g., Br, Cl). Exemplary acyl halides include acyl chloride (—C(O)Cl) and acyl bromide (—C(O)Br).

In accordance with convention used in the art:

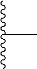

is used in structural formulas herein to depict the bond that is the point of attachment of the moiety, functional group, or substituent to the core, parent, or backbone structure, such as an antigen binding domain of the present invention.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

When a bond to a substituent is shown to cross a bond connecting two atoms in a ring, then such substituent can be bonded to any atom on the ring.

In certain embodiments, the radioconjugate is ²²⁵Ac-DOTA-h11B6, also referred to as Actinium 225-1,4,7,10-tetraazacyclododecane-1,4,7,10,tetraacetic acid-h11B6. As used herein, ²²⁵Ac-DOTA-h11B6 is a radioconjugate that comprises ²²⁵Ac chelated to DOTA wherein said DOTA is conjugated to h11B6, optionally via a linker. As used herein, ¹¹¹In-DOTA-h11B6 is a radioconjugate that comprises ¹¹¹In chelated to DOTA wherein said DOTA is conjugated to h11B6, optionally via a linker.

In certain embodiments, the radioconjugate may be represented by the following compound or a variant thereof:

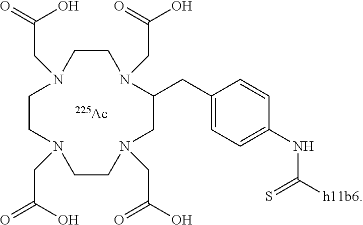

In certain embodiments, the radioconjugate is ²²⁵Ac-TOPA-h11B6. As used herein, ²²⁵Ac-TOPA-h11B6 is a radioconjugate that comprises ²²⁵Ac chelated to TOPA wherein said TOPA is conjugated to h11B6, optionally via a linker. In an embodiment, the ²²⁵Ac-TOPA-h11B6 radioconjugate may be represented by the following compound or a variant thereof, which may also be referred to as a TOPA-[C7]-phenylthiourea-h11B6 antibody conjugate:

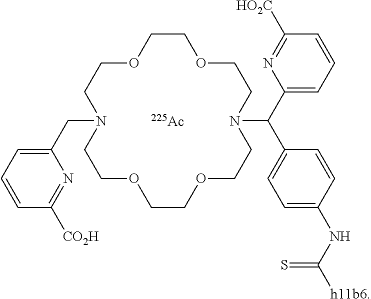

In the TOPA-[C7]-phenylthiourea-h11B6 antibody conjugate depicted above, the structure does not show the lysine residue of h11b6 that is linked to the phenylthiourea moiety, which is depicted in FIG. 6B and FIG. 6C.

### Pharmaceutical Compositions and Methods of Use

Embodiments of the present invention provide methods of treating cancer in a patient, the method comprising administering to the patient a therapeutically effective amount of a radioconjugate. According to an embodiment, the method comprises administering to the patient a therapeutically effective amount of a pharmaceutical composition comprising a radioconjugate and one or more pharmaceutically acceptable excipients.

Embodiments of the present invention are particularly useful in treating patients that have been diagnosed with prostate cancer; for example, patients that have late-stage prostate cancer. According to an embodiment, the cancer is non-localized prostate cancer. According to another embodiment, the cancer is metastatic prostate cancer. According to another embodiment, the cancer is castration-resistant prostate cancer (CRPC). According to another embodiment, the cancer is metastatic castration-resistant prostate cancer (mCRPC). According to another embodiment, the cancer is mCRPC with adenocarcinoma. According to particular embodiments, testosterone castrate levels of the patient are about 50 ng/dL or less. According to additional embodiments, the patient had prior exposure to at least one androgen receptor (AR) targeted therapy; for example, abiraterone acetate, enzalutamide, apalutamide, darolutamide, or combinations of any of the foregoing. According to additional embodiments, the patient had prior chemotherapy; for example, the chemotherapy involved administration of taxane. According to another embodiment, the patient had prior orchiectomy or medical castration. According to another embodiment, the patient is receiving ongoing androgen deprivation therapy with a gonadotropin releasing hormone (GnRH) agonist or antagonist.

In accordance with embodiments of the treatment methods described herein, the radioconjugate administered to the patient comprises at least one radiometal complex conjugated to an antibody, or an antigen binding fragment, with binding specificity for hK2. Preferably, the radioconjugate comprises at least one radiometal complex conjugated to an antibody with binding specificity for hK2. In preferred embodiments, the radiometal complex comprises ²²⁵Ac.

According to an embodiment, the radiometal in the pharmaceutical composition is ²²⁵Ac and provides a targeted radioactivity from about 50 μCi to about 350 μCi per dose of the pharmaceutical composition. According to additional embodiments, the radiometal in the pharmaceutical composition provides a targeted radioactivity from about 50 μCi to about 300 μCi, or from about 50 μCi to about 250 μCi, or from about 50 μCi to about 240 μCi, or from about 50 μCi to about 230 μCi, or from about 50 μCi to about 220 μCi, or from about 50 μCi to about 210 μCi, or from about 50 μCi to about 200 μCi, or from about 50 μCi to about 175 μCi, or from about 50 μCi to about 150 μCi, or from about 50 μCi to about 125 μCi, or from about 50 μCi to about 100 μCi, or from about 100 μCi to about 300 μCi, or from about 100 μCi to about 250 μCi, or from about 100 μCi to about 240 μCi, or from about 100 μCi to about 230 μCi, or from about 100 μCi to about 220 μCi, or from about 100 μCi to about 210 μCi, or from about 100 μCi to about 200 μCi, or from about 100 μCi to about 175 μCi, or from about 100 μCi to about 150 μCi, or from about 150 μCi to about 300 μCi, or from about 150 μCi to about 250 μCi, or from about 175 μCi to about 225 μCi per dose of the pharmaceutical composition.

According to an embodiment, the radiometal in the pharmaceutical composition is ²²⁵Ac and provides a targeted radioactivity from about 50 μCi to about 500 μCi per dose of the pharmaceutical composition. According to additional embodiments, the radiometal in the pharmaceutical composition provides a targeted radioactivity from about 50 μCi to about 450 μCi, or from about 50 μCi to about 400 μCi, or from about 50 μCi to about 350 μCi, or from about 100 μCi to about 500 μCi, or from about 100 μCi to about 450 μCi, or from about 100 μCi to about 400 μCi, or from about 100 μCi to about 350 μCi, or from about 150 μCi to about 500 μCi, or from about 150 μCi to about 450 μCi, or from about 150 μCi to about 400 μCi, or from about 150 μCi to about 350 μCi, or from about 200 μCi to about 500 μCi, or from about 200 μCi to about 450 μCi, or from about 200 μCi to about 400 μCi, or from about 200 μCi to about 350 μCi per dose of the pharmaceutical composition.

According to an embodiment, the radiometal in the pharmaceutical composition is ²¹⁵Ac and provides a targeted specific activity from about 50 μCi to about 350 μCi per about 2 mg total antibody in the pharmaceutical composition. According to additional embodiments, the radiometal in the pharmaceutical composition provides a targeted specific activity from about 50 μCi to about 300 μCi, or from about 50 μCi to about 250 μCi, or from about 50 μCi to about 240 μCi, or from about 50 μCi to about 230 μCi, or from about 50 μCi to about 220 μCi, or from about 50 μCi to about 210 μCi, or from about 50 μCi to about 200 μCi, or from about 50 μCi to about 175 μCi, or from about 50 μCi to about 150 μCi, or from about 50 μCi to about 125 μCi, or from about 50 μCi to about 100 μCi, or from about 100 μCi to about 300 μCi, or from about 100 μCi to about 250 μCi, or from about 100 μCi to about 240 μCi, or from about 100 μCi to about 230 μCi, or from about 100 μCi to about 220 μCi, or from about 100 μCi to about 210 μCi, or from about 100 μCi to about 200 μCi, or from about 100 μCi to about 175 μCi, or from about 100 μCi to about 150 μCi, or from about 150 μCi to about 300 μCi, or from about 150 μCi to about 250 μCi, or from about 175 μCi to about 225 μCi radioactivity per about 2 mg total antibody in the pharmaceutical composition. According to additional embodiments, the radiometal in the pharmaceutical composition provides a targeted specific activity of about 50 μCi, or about 100 μCi, or about 150 μCi, or about 175 μCi, or about 200 μCi, or about 225 μCi, or about 250 μCi, or about 275 μCi, or about 300 μCi per about 2 mg total antibody in the pharmaceutical composition. It should be appreciated, for example, that an amount of 300 μCi per 2 mg of antibody is equivalent to 150 μCi/mg of antibody, 200 μCi per 2 mg of antibody is equivalent to 100 μCi/mg of antibody, etc.

According to another embodiment, the radiometal in the pharmaceutical composition is ²²⁵Ac and provides a targeted specific activity from about 50 μCi to about 350 μCi per between about 2 mg and about 10 mg total antibody in the pharmaceutical composition (e.g., per about 2 mg, or about 4 mg, or about 6 mg, or about 8 mg, or about 10 mg total antibody in the pharmaceutical composition). According to additional embodiments, the radiometal in the pharmaceutical composition provides a targeted specific activity from about 50 μCi to about 300 μCi, or from about 50 μCi to about 250 μCi, or from about 50 μCi to about 240 μCi, or from about 50 μCi to about 230 μCi, or from about 50 μCi to about 220 μCi, or from about 50 μCi to about 210 μCi, or from about 50 μCi to about 200 μCi, or from about 50 μCi to about 175 μCi, or from about 50 μCi to about 150 μCi, or from about 50 μCi to about 125 μCi, or from about 50 μCi to about 100 μCi, or from about 100 μCi to about 300 μCi, or from about 100 μCi to about 250 μCi, or from about 100 μCi to about 240 μCi, or from about 100 μCi to about 230 μCi, or from about 100 μCi to about 220 μCi, or from about 100 μCi to about 210 μCi, or from about 100 μCi to about 200 μCi, or from about 100 μCi to about 175 μCi, or from about 100 μCi to about 150 μCi, or from about 150 μCi to about 300 μCi, or from about 150 μCi to about 250 μCi, or from about 175 μCi to about 225 μCi radioactivity per between about 2 mg and about 10 mg total antibody in the pharmaceutical composition (e.g., per about 2 mg, or about 4 mg, or about 6 mg, or about 8 mg, or about 10 mg total antibody in the pharmaceutical composition). According to additional embodiments, the radiometal in the pharmaceutical composition provides a targeted specific activity of about 50 μCi, or about 100 μCi, or about 150 μCi, or about 175 μCi, or about 200 μCi, or about 225 μCi, or about 250 μCi, or about 275 μCi, or about 300 μCi or about 350 μCi per between about 2 mg and about 10 mg total antibody in the pharmaceutical composition (e.g., per about 2 mg, or about 4 mg, or about 6 mg, or about 8 mg, or about 10 mg total antibody in the pharmaceutical composition).

According to another embodiment, the radiometal in the pharmaceutical composition is ²²⁵Ac and provides a targeted specific activity from about 50 μCi to about 500 μCi per between about 2 mg and about 10 mg total antibody in the pharmaceutical composition (e.g., per about 2 mg, or about 4 mg, or about 6 mg, or about 8 mg, or about 10 mg total antibody in the pharmaceutical composition). According to additional embodiments, the radiometal in the pharmaceutical composition provides a targeted specific activity from about 50 μCi to about 450 μCi, or from about 50 μCi to about 400 μCi, or from about 50 μCi to about 350 μCi, or from about 100 μCi to about 500 μCi, or from about 100 μCi to about 450 μCi, or from about 100 μCi to about 400 μCi, or from about 100 μCi to about 350 μCi, or from about 150 μCi to about 500 μCi, or from about 150 μCi to about 450 μCi, or from about 150 μCi to about 400 μCi, or from about 150 μCi to about 350 μCi, or from about 200 μCi to about 500 μCi, or from about 200 μCi to about 450 μCi, or from about 200 μCi to about 400 μCi, or from about 200 μCi to about 350 μCi radioactivity per between about 2 mg and about 10 mg total antibody in the pharmaceutical composition (e.g., per about 2 mg, or about 4 mg, or about 6 mg, or about 8 mg, or about 10 mg total antibody in the pharmaceutical composition). According to additional embodiments, the radiometal in the pharmaceutical composition provides a targeted specific activity of about 350 μCi, or about 375 μCi, or about 400 μCi, or about 425 μCi, or about 450 μCi, or about 475 μCi, or about 500 μCi per between about 2 mg and about 10 mg total antibody in the pharmaceutical composition (e.g., per about 2 mg, or about 4 mg, or about 6 mg, or about 8 mg, or about 10 mg total antibody in the pharmaceutical composition).

According to an embodiment, the radiometal in the pharmaceutical composition is ²²⁵Ac and the targeted radioactive concentration of the pharmaceutical composition is from about 1 μCi/mL to about 100 μCi/mL, or from about 5 μCi/mL to about 75 μCi/mL, or from about 10 μCi/mL to about 60 μCi/mL, or from about 12.5 μCi/mL to about 50 μCi/mL, or about 12.5 μCi/mL, or about 25 μCi/mL, or about 37.5 μCi/mL, or about 50 μCi/mL.

As used herein, “time of dosing” refers to the time at which a patient is administered a dose of the pharmaceutical composition comprising the radioconjugate (e.g., whether as a single administration or in multiple administrations of more than one sub-dose). Due to the decay of ²²⁵Ac, the amount of radioactivity provided by ²²⁵Ac in the pharmaceutical composition decreases from the time of manufacture to the time of dosing, i.e., from the approximate time that ²²⁵Ac is chelated to the conjugate intermediate to form the radioconjugate during the manufacturing process to the time that the radioconjugate is administered to a patient. According to an example, if the radioactivity provided by ²²⁵Ac in the pharmaceutical composition is about 264 μCi at the approximate time that ²²⁵Ac is chelated to the conjugate intermediate to form the radioconjugate (e.g., after the radioconjugate is formed and purified), the radioactivity about 96 hours later at the time of dosing may be about 200 μCi. The decay and therefore the amount of ²²⁵Ac at any given time may be calculated based on the initial amount of activity measured at time zero, the amount of time elapsed and the half-life of ²²⁵Ac.

As used herein, a “targeted” specific activity or “targeted” radioactivity or “targeted” radioactive concentration of the radiometal refers to the amount of specific activity or radioactivity or radioactive concentration, respectively, that is calculated to be present in a dose of pharmaceutical composition at the anticipated time of administration to the patient, e.g., based on the amount of radiometal present in the composition at the time of manufacture and the amount of time (and accompanying radiometal decay) expected between manufacture and administration to the patient. It should be appreciated that the actual specific activity or radioactivity or radioactive concentration at the time of dosing may vary slightly from the targeted specific activity or radioactivity or radioactive concentration, respectively (for example, in the event that the actual time of administration to a patient differs slightly from the anticipated time of administration).

According to particular embodiments, the pharmaceutical composition also contains non-radiolabeled antibody. For example, a composition comprising non-radiolabeled antibody may be combined with a composition comprising the radioconjugate, in order to dilute the radioconjugate composition to a desired dose of radioactivity. The term “non-radiolabeled antibody” as used herein refers to an antibody or an antibody-chelator complex that is not conjugated to a radiometal. According to particular embodiments, the non-radiolabeled antibody present in the composition is a conjugate intermediate, such as DOTA-mAb (for example, DOTA-h11B6). Preferably, the non-radiolabeled antibody comprises the same antibody as the radioconjugate contained in the composition; for example, the pharmaceutical composition may comprise a quantity of ²²⁵Ac-DOTA-h11B6 and a quantity of DOTA-h11B6. Alternatively, the pharmaceutical composition may comprise a quantity of ²²⁵Ac-TOPA-h11B6 and a quantity of TOPA-h11B6. As used herein, “total antibody” refers to the total amount of antibody in a pharmaceutical composition; for example, the total antibody may include (a) the amount of antibody conjugated to the radiometal complex and (b) an amount of non-radiolabeled antibody, such as conjugate intermediate. The targeted total antibody refers to the amount of antibody that is calculated to be present in a dose of pharmaceutical composition at the anticipated time of administration to the patient. According to certain embodiments, the total amount of antibody (radiolabeled and non-radiolabeled) does not exceed about 10 mg, or about 9 mg, or about 8 mg, or about 7 mg, or about 6 mg, or about 5 mg, or about 4 mg, or about 3 mg, or about 2 mg in the composition.

According to certain embodiments, a method of making a pharmaceutical composition of the present invention comprises combining a first intermediate composition and a second intermediate composition to form the pharmaceutical composition, wherein: the first intermediate composition comprises the radioconjugate, and the second intermediate composition comprises a conjugate intermediate and does not contain any radioconjugate. According to certain embodiments, the first intermediate composition and the second intermediate composition comprise the same pharmaceutically acceptable excipients.

According to particular embodiments, the pharmaceutical composition comprises from about 0.1 mg to about 5 mg of total antibody, or from about 0.1 mg to about 4 mg of total antibody, or from about 0.1 mg to about 3 mg of total antibody, or from about 0.1 mg to about 4 mg of total antibody, from about 0.1 mg to about 3 mg of total antibody, or from about 0.1 mg to about 2 mg of total antibody, or from about 0.5 mg to about 5 mg of total antibody, or from about 0.5 mg to about 4 mg of total antibody, or from about 0.5 mg to about 3 mg of total antibody, or from about 0.5 mg to about 3.5 mg of total antibody, or from about 0.5 mg to about 4 mg of total antibody, or from about 1 mg to about 10 mg of total antibody, or from about 1 mg to about 7 mg of total antibody, or from about 1 mg to about 5 mg of total antibody, or from about 1 mg to about 4 mg of total antibody, or from about 1 mg to about 3 mg of total antibody, or from about 1.5 to about 2.5 mg of total antibody, or about 1.1 or about 1.2 mg of total antibody, or about 1.3 mg of total antibody, or about 1.4 mg of total antibody, or about 1.5 mg of total antibody, or about 1.6 mg of total antibody, or about 1.7 mg of total antibody, or about 1.8 mg of total antibody, or about 1.9 mg of total antibody, or about 2 mg of total antibody, or about 2.1 mg of total antibody, or about 2.2 mg of total antibody, or about 2.3 mg of total antibody, or about 2.4 mg of total antibody, or about 2.5 mg of total antibody, or about 2.6 mg of total antibody, or about 2.7 mg of total antibody, or about 2.8 mg of total antibody, or about 2.9 mg of total antibody.

According to particular embodiments, the dose of the pharmaceutical composition has a volume from about 1 mL to about 20 mL, or about 1 mL to about 10 mL, or about 2 mL to about 6 mL, or about 3 mL to about 5 mL, or about 4 mL. According to an embodiment, the dose of the pharmaceutical composition comprises about 2 mg of total antibody per about 4 mL of the dose (i.e., about 1 mg of total antibody per about 2 mL of the dose). As noted herein, the dose may be administered as multiple sub-doses; for example, an 8-mL dose may be administered as two 4-mL sub-doses. In an embodiment, the subject is administered two 4-mL sub-doses, which each sub-dose containing 2 mg antibody, for a total of 4 mg antibody per 8-mL dose.

According to particular embodiments, the pharmaceutical composition comprises total antibody in an amount of about 0.01-5.0 mg/mL, or about 0.01-4.0 mg/mL, or about 0.01-3.0 mg/mL, about 0.01-2.0 mg/mL, or about 0.01-1.0 mg/mL, about 0.1-5.0 mg/mL, or about 0.1-4.0 mg/mL, or about 0.1-3.0 mg/mL, about 0.1-2.0 mg/mL, or about 0.1-1.0 mg/mL, about 0.3-0.7 mg/mL, or about 0.4-0.6 mg/mL, or about 0.5 mg/mL.

According to one embodiment, the targeted total antibody concentration in a vial containing a dose of the pharmaceutical composition is about 0.5±0.1 mg/mL; for example, in a vial containing ²²⁵Ac-DOTA-h11B6 and DOTA-h11B6. In one embodiment, if a dose contains about 4 mL of pharmaceutical composition, the targeted total antibody concentration in the dose is about 0.5 mg/mL, the targeted radioactivity for the dose is about 50 μCi, and the targeted radioactive concentration is about 12.5 μCi/mL (e.g., 12.5 μCi/mL+10%). In another embodiment, if a dose contains about 4 mL of pharmaceutical composition, the targeted total antibody concentration in the dose is about 0.5 mg/mL, the targeted radioactivity for the dose is about 100 μCi, and the targeted radioactive concentration is about 25 μCi/mL (e.g., 25 μCi/mL 10%). In another embodiment, if a dose contains about 4 mL of pharmaceutical composition, the targeted total antibody concentration in the dose is about 0.5 mg/mL, the targeted radioactivity for the dose is about 150 μCi, and the targeted radioactive concentration is about 37.5 μCi/mL (e.g., 37.5 μCi/mL±10%). In another embodiment, if a dose contains about 4 mL of pharmaceutical composition, the targeted total antibody concentration in the dose is about 0.5 mg/mL (about 2 mg total antibody), the targeted radioactivity for the dose is about 200 μCi, and the targeted radioactive concentration is about 50 μCi/mL (e.g., 50 μCi/mL+10%). In another embodiment, if a dose contains about 8 mL of pharmaceutical composition, the targeted total antibody concentration in the dose is about 0.5 mg/mL (about 4 mg total antibody), the targeted radioactivity for the dose is about 300 μCi, and the targeted radioactive concentration is about 37.5 μCi/mL (e.g., 37.5 μCi/mL±10%).

According to further embodiments, the total antibody is present in the pharmaceutical composition at a concentration of about 0.1 mg/mL to about 1 mg/mL. In some embodiments, the concentration of total antibody in the pharmaceutical composition is about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1, about 0.3 to about 0.9, about 0.3 to about 0.8, about 0.3 to about 0.7, about 0.3 to about 0.6, about 0.3 to about 0.5, about 0.3 to about 0.4, about 0.4 to about 1, about 0.4 to about 0.9, about 0.4 to about 0.8, about 0.4 to about 0.7, about 0.4 to about 0.6, about 0.4 to about 0.5, about 0.5 to about 1, about 0.5 to about 0.9, about 0.5 to about 0.8, about 0.5 to about 0.7, about 0.5 to about 0.6, about 0.6 to about 1, about 0.6 to about 0.9, about 0.6 to about 0.8, about 0.6 to about 0.7, about 0.7 to about 1, about 0.7 to about 0.9, about 0.7 to about 0.8, about 0.8 to about 1, about 0.8 to about 0.9, or about 0.9 to about 1 mg/mL. In other embodiments, the pharmaceutical composition contains about 0.5 mg/mL of total antibody. In further embodiments, the pharmaceutical composition contains a total of about 0.1 mg/mL to about 1 mg/mL of ²²⁵Ac-DOTA-h11B6 and DOTA-h11B6. In further embodiments, the pharmaceutical composition contains a total of about 0.5 mg/mL of ²²⁵Ac-DOTA-h11B6 and DOTA-h11B6. In further embodiments, the pharmaceutical composition contains a total of about 0.1 mg/mL to about 1 mg/mL of ²²⁵Ac-TOPA-h11B6 and TOPA-h11B6. In further embodiments, the pharmaceutical composition contains a total of about 0.5 mg/mL of ²²⁵Ac-TOPA-h11B6 and TOPA-h11B6.

Pharmaceutical compositions of the present invention may be administered via any suitable route known to those skilled in the art. For example, the compositions may be administered parenterally. Non-limiting examples of routes of administration include intravenous (IV), intramuscular or subcutaneous, or they may be administered by infusion techniques. In certain aspects, the methods of treatment herein comprise injecting the pharmaceutical composition intravenously.

According to an embodiment, a pharmaceutical composition of the present invention is provided in a single-use, sterile solution for injection. The composition is preferably refrigerated until the time of injection in a sealed vial; for example, in a cyclic olefin polymer vial closed with a latex-free stopper and aluminum seal.

Pharmaceutical compositions of the present invention may be administered to the patient by a healthcare professional. In some embodiments, the pharmaceutical composition is administered once every about 2, about 3, about 4, about 5, about 6, about 7, about 8, about 9, about 10, about 11, about 12, about 13, about 14, about 15, or about 16 weeks. In some embodiments, the pharmaceutical composition is administered once every about 4 to about 12, about 4 to about 10, about 4 to about 8, about 4 to about 6, about 6 to about 12, about 6 to about 10, about 6 to about 8, about 8 to about 12, about 8 to about 10, or about 10 to about 12 weeks. In certain aspects, the pharmaceutical composition is administered to the patient once every about 4 weeks. In certain aspects, the pharmaceutical composition is administered to the patient once every about 6 weeks. In other aspects, the pharmaceutical composition is administered to the patient once every about 8 weeks. In certain aspects, the pharmaceutical composition is administered to the patient once every about 10 weeks. In further aspects, the pharmaceutical composition is administered to the patient once every about 12 weeks. According to certain embodiments, the patient is administered from about 2 to about 12 doses, or about 2 to about 10 doses, or from about 2 to about 8 doses, or from about 2 to about 6 doses, or from about 2 to about 4 doses. According to an embodiment, a patient's dosing regimen comprises administering at least 2 doses, or at least 3 doses, or at least 4 doses, or at least 5 doses, or at least 6 doses, wherein one dose is administered every 8 weeks. According to an embodiment, a patient's dosing regimen comprises administering 4 total doses, wherein one dose is administered every 8 weeks. Alternatively, a patient's dosing regimen comprises administering 5 or 6 or 7 or 8 or 9 or 10 or 11 or 12 total doses, wherein one dose is administered once every 8 weeks. In still further embodiments, a patient's dosing regimen may continue so that it includes more than 12 total doses.

A dose of a pharmaceutical composition of the present invention may be administered to the patient by a single administration, or by administering the dose in multiple administrations of more than one sub-dose (e.g., by administering the dose in multiple subdivisions of the dose). Alternatively, the dose may be provided as a continuous infusion over a prolonged period.

It will be appreciated by persons skilled in the art that pharmaceutical compositions of the present invention may be administered alone or in combination with one or more additional therapeutic agents or imaging agents or modalities as determined by the attending physician. A pharmaceutical composition of the present invention may be administered to the patient before or concurrently with other therapeutic modalities for the treatment of prostate cancer.

Preferably, pharmaceutical compositions of the present invention are in the form of a sterile aqueous solution which may contain other substances to make the solution isotonic with blood and/or to provide a suitable pH. In some embodiments, the pH of the aqueous solution is about 4 to about 7, about 4.5 to about 6.5, about 5 to about 6, or about 5.5. In other embodiments, the pH of the aqueous solution is about 5, about 5.1, about 5.2, about 5.3, about 5.4, about 5.5, about 5.6, about 5.7, about 5.8, about 5.9, or about 6. In further embodiments, the pH of the aqueous solution is about 5.5.

As noted herein, due to the decay of ²²⁵Ac, the amount of radioactivity provided by ²²⁵Ac in a dose of the pharmaceutical composition decreases from the time of manufacture (from the approximate time that ²²⁵Ac is chelated to the conjugate intermediate to form the radioconjugate) to the time that the dose is administered to a patient. Preferably, the radioconjugate is labeled with a sufficient amount of ²²⁵Ac during manufacture to account for the decrease in specific activity of ²²⁵Ac that is estimated to occur between the approximate time of chelation and the time of dosing a patient. It is also preferable to limit the amount of time between formation of the radioconjugate (via chelation of ²²⁵Ac) to administration of the dose to the patient.

According to particular embodiments, a pharmaceutical composition of the present invention is administered to the patient within about 7 days (168 hours) from chelation of the radiometal to a conjugate intermediate to form the radioconjugate, or within about 144 hours, or within about 120 hours, or within about 96 hours, or within about 72 hours, or within about 48 hours, or within about 24 hours from chelation of the radiometal to a conjugate intermediate to form the radioconjugate. According to particular embodiments, a method of the present invention comprises administering the pharmaceutical composition to the patient (i.e., the time of dosing occurs) within about 120 hours or less from chelation of the radiometal to a conjugate intermediate to form the radioconjugate. According to particular embodiments, a method of the present invention comprises administering the pharmaceutical composition to the patient (i.e., the time of dosing occurs) within about 96 hours or less from chelation of the radiometal to a conjugate intermediate to form the radioconjugate. According to particular embodiments, a method of the present invention comprises administering the pharmaceutical composition to the patient (i.e., the time of dosing occurs) within about 72 hours or less from chelation of the radiometal to a conjugate intermediate to form the radioconjugate.

According to particular embodiments, radioconjugates of the present invention are administered in admixture with one or more pharmaceutically acceptable excipients. The terms “pharmaceutical composition” and “pharmaceutical formulation” are used interchangeably throughout this disclosure. The pharmaceutical compositions may be prepared using techniques known in the art. In particular embodiments, the pharmaceutical compositions are sufficiently storage stable and suitable for administration to humans.

The excipients may be selected by one skilled in the art and may take a wide variety of forms depending upon the desired route of administration. For example, for parenteral administration, the excipients may include sterile water, and other ingredients may be added to increase solubility and preservation of the composition. Injectable suspensions or solutions may also be prepared utilizing excipients that comprise aqueous carriers and/or appropriate additives such as, solubilizers and preservatives.

In some embodiments, the excipients comprise a buffer, which is an aqueous solution preferably containing an acid-base mixture with the purpose of stabilizing the pH of a solution.

Examples of buffers include, without limitation, Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO, or TES. In some embodiments, the buffer is Trizma. In other embodiments, the buffer is Bicine. In further embodiments, the buffer is Tricine. In still other embodiments, the buffer is MOPS. In yet further embodiments, the buffer is MOPSO. In other embodiments, the buffer is MOBS. In further embodiments, the buffer is Tris. In still other embodiments, the buffer is Hepes. In yet further embodiments, the buffer is HEPBS. In other embodiments, the buffer is MES. In further embodiments, the buffer is phosphate. In still other embodiments, the buffer is carbonate. In yet further embodiments, the buffer is acetate. In yet other embodiments, the buffer is citrate. In still further embodiments, the buffer is glycolate. In other embodiments, the buffer is lactate. In further embodiments, the buffer is borate. In still other embodiments, the buffer is ACES. In yet further embodiments, the buffer is ADA. In other embodiments, the buffer is tartrate. In further embodiments, the buffer is AMP. In yet other embodiments, the buffer is AMPD. In still further embodiments, the buffer is AMPSO. In other embodiments, the buffer is BES. In further embodiments, the buffer is CABS. In yet other embodiments, the buffer is cacodylate. In still further embodiments, the buffer is CHES. In other embodiments, the buffer is DIPSO. In further embodiments, the buffer is EPPS. In still other embodiments, the buffer is ethanolamine. In yet further embodiments, the buffer is glycine. In other embodiments, the buffer is HEPPSO. In further embodiments, the buffer is imidazole. In still other embodiments, the buffer is imidazolelactic acid. In yet further embodiments, the buffer is PIPES. In other embodiments, the buffer is SSC. In further embodiments, the buffer is SSPE. In still other embodiments, the buffer is POPSO. In yet further embodiments, the buffer is TAPS. In other embodiments, the buffer is TABS. In further embodiments, the buffer is TAPSO. In other embodiments, the buffer is TES. The buffer is desirably provided in a concentration that obtains the desired pH. In some aspects, the concentration of the buffer is about 10 to about 50 mM. In other aspects, the pH of the buffer is about 20 to about 50, about 25 to about 50, about 30 to about 50, about 35 to about 50, about 40 to about 50, about 45 to about 50, about 20 to about 45, about 25 to about 45, about 30 to about 45, about 35 to about 45, about 40 to about 45, about 20 to about 40, about 25 to about 40, about 30 to about 40, about 35 to about 40, about 20 to about 35, about 25 to about 35, about 30 to about 35, about 20 to about 30, about 25 to about 30, or about 20 to about 25 mM. In further aspects, the concentration of the buffer is about 24 to about 28, about 25 to about 28, about 25 to about 27, about 26 to about 28, or about 26 to about 27 mM. In still other aspects, the concentration of the buffer is about 25 mM. In still other aspects, the concentration of the buffer is about 26.75 mM. In an embodiment, the buffer comprises acetate.

In additional embodiments, the excipients comprise a diluent. The term “diluent” refers to aqueous or non-aqueous solutions with the purpose of diluting the pharmaceutical composition. For example, a diluent may comprise one or more of saline, water, polyethylene glycol, propylene glycol, ethanol or oils (such as safflower oil, corn oil, peanut oil, cottonseed oil or sesame oil). In certain embodiments, the diluent is water. Additional non-limiting examples of diluents include sterile water, saline of varying concentrations (NS/0.9%, ½NS/0.45%), dextrose of varying concentrations (D5W, D10W), or dextrose+saline (½NSD5W)

The pharmaceutical compositions may be subjected to conventional pharmaceutical operations such as sterilization and/or may contain conventional adjuvants. Pharmaceutical compositions may also contain aqueous and non-aqueous sterile injection solutions which may contain antioxidants, buffers, bacteriostats and/or solutes which render the formulation isotonic with the blood of the intended recipient.

In still other embodiments, the excipients may comprise one or more of a binder, carbohydrate, coating agent, coloring agent, disintegrating agent, dispersing agent, emulsifier, filler, flavoring agent, granulating agent, lipid, lubricant, mineral, polymer, preservative, radioprotectant, solubilizing agent, stabilizer, suspending agent, sweetener, thickening agent, wetting agent, or combinations thereof.

According to particular embodiments, the excipients comprise at least one radioprotectant. In certain embodiments, the pharmaceutical composition comprises: a radioconjugate and one or more pharmaceutically acceptable excipients, wherein: the radioconjugate comprises at least one radiometal complex conjugated to an antibody, or an antigen binding fragment, with binding specificity for hK2 (e.g., h11B6 or a variant thereof), and the one or more pharmaceutically acceptable excipients comprise one or more radioprotectants.

Examples of radioprotectants include, without limitation, sodium ascorbate, gentisic acid, or a combination thereof. According to an embodiment, the composition comprises sodium ascorbate. According to an alternative embodiment, the composition comprises gentisic acid.

In other embodiments, the excipients comprise one or more surfactants. Non-limiting examples of surfactants include polysorbates and poloxamers, such as polysorbate 20, polysorbate 80 and poloxamer 188. According to an embodiment, the excipients comprise polysorbate 20. In further embodiments, the excipients comprise sodium ascorbate, polysorbate 20, or a combination thereof. In some embodiments, the pharmaceutical composition contains the radioconjugate and sodium ascorbate. In other embodiments, the pharmaceutical composition contains polysorbate 20. In further embodiments, the pharmaceutical composition contains sodium ascorbate and polysorbate 20. In some embodiments, the pharmaceutical composition contains the radioconjugate and gentisic acid. In further embodiments, the pharmaceutical composition contains gentisic acid and polysorbate 20.

The amount of the excipient(s) is selected based on the desired route of administration, the patient and the particular excipient(s) in the pharmaceutical composition. In preferred embodiments, the amount of sodium ascorbate present in the pharmaceutical composition inhibits degradation of the radioconjugate. Desirably, sodium ascorbate inhibits degradation of the radioconjugate as compared to a composition that does not contain sodium ascorbate, e.g., as measured by spectroscopic methods such as high performance liquid chromatography, nuclear magnetic resonance, mass spectra, or elemental analysis, among others. In other embodiments, the pharmaceutical composition contains about 0.05 to about 5.0 w/v % of sodium ascorbate and/or gentisic acid. In further embodiments, the pharmaceutical composition contains about 0.1 to about 5, about 0.1 to about 4, about 0.1 to about 3, about 0.1 to about 2, about 0.1 to about 1, about 0.2 to about 1, about 0.3 to about 1, about 0.4 to about 1, about 0.5 to about 1, about 0.6 to about 1, about 0.7 to about 1, about 0.8 to about 1, about 0.9 to about 1, about 0.1 to about 0.9, about 0.2 to about 0.9, about 0.3 to about 0.9, about 0.4 to about 0.9, about 0.5 to about 0.9, about 0.6 to about 0.9, about 0.7 to about 0.9, about 0.8 to about 0.9, about 0.1 to about 0.8, about 0.2 to about 0.8, about 0.3 to about 0.8, about 0.4 to about 0.8, about 0.5 to about 0.8, about 0.6 to about 0.8, about 0.7 to about 0.8, about 0.1 to about 0.7, about 0.2 to about 0.7, about 0.3 to about 0.6, about 0.4 to about 0.6, about 0.5 to about 0.6, about 0.1 to about 0.5, about 0.2 to about 0.5, about 0.3 to about 0.5, about 0.4 to about 0.5, about 0.1 to about 0.4, about 0.2 to about 0.4, about 0.3 to about 0.4, about 0.1 to about 0.3, about 0.2 to about 0.3, or about 0.1 to about 0.2 w/v % of sodium ascorbate. In yet other embodiments, the pharmaceutical composition contains about 0.1, about 0.2, about 0.3, about 0.4, about 0.5, about 0.6 about 0.7, about 0.8, about 0.9, or about 1 w/v % of sodium ascorbate. In still further embodiments, the pharmaceutical composition contains about 0.5 w/v % of sodium ascorbate.

In other embodiments, the pharmaceutical composition contains about 0.005 to about 0.15 w/v %, or about 0.005 to about 0.12 w/v %, or about 0.005 to about 0.1 w/v %, or about 0.005 to about 0.08 w/v %, or about 0.005 to about 0.06 w/v %, or about 0.005 to about 0.12 w/v %, or about 0.005 to about 0.04 w/v % of polysorbate 20. In certain aspects, the pharmaceutical composition contains about 0.01 to about 0.12, about 0.02 to about 0.12, about 0.03 to about 0.12, about 0.04 to about 0.12, about 0.05 to about 0.12, about 0.06 to about 0.12, about 0.07 to about 0.12, about 0.08 to about 0.12, about 0.09 to about 0.12, about 0.01 to about 0.1, about 0.02 to about 0.1, about 0.03 to about 0.1, about 0.04 to about 0.1, about 0.05 to about 0.1, about 0.06 to about 0.1, about 0.07 to about 0.1, about 0.08 to about 0.1, about 0.09 to about 0.1, about 0.01 to about 0.09, about 0.02 to about 0.09, about 0.03 to about 0.09, about 0.04 to about 0.09, about 0.05 to about 0.09, about 0.06 to about 0.09, about 0.07 to about 0.09, about 0.08 to about 0.09, about 0.01 to about 0.08, about 0.02 to about 0.08, about 0.03 to about 0.08, about 0.04 to about 0.08, about 0.05 to about 0.08, about 0.06 to about 0.08, about 0.07 to about 0.08, about 0.01 to about 0.07, about 0.02 to about 0.07, about 0.03 to about 0.06, about 0.04 to about 0.06, about 0.05 to about 0.06, about 0.01 to about 0.05, about 0.02 to about 0.05, about 0.03 to about 0.05, about 0.04 to about 0.05, about 0.01 to about 0.04, about 0.02 to about 0.04, about 0.03 to about 0.04, about 0.01 to about 0.03, about 0.02 to about 0.03, or about 0.01 to about 0.02 w/v % of polysorbate 20. In other aspects, the pharmaceutical composition contains about 0.01, about 0.02, about 0.03, about 0.04, about 0.05, about 0.06 about 0.07, about 0.08, about 0.09, about 0.1, about 0.11, about 0.12, about 0.13, about 0.14, or about 0.15 w/v % of polysorbate 20. In further aspects, the pharmaceutical composition contains about 0.04 w/v % of polysorbate 20.

According to certain embodiments, the pharmaceutical composition does not contain any preservatives.

According to certain embodiments, the pharmaceutical composition does not contain any sucrose; in particular, the pharmaceutical composition may not contain any sucrose when the radiometal is ²²⁵Ac, for example, when the radioconjugate is ²²⁵Ac-DOTA-h11B6. According to certain embodiments, the inclusion of sucrose in a composition containing ²²⁵Ac results in the formation of radiolytic degradants, as described herein. Thus, in certain embodiments, sucrose may be excluded or limited to small amounts, e.g., less than 1%, less than 0.5%, less than 0.1%, less than 0.05%, or less than 0.01%.

According to certain embodiments, the pharmaceutical composition does not contain any dextrins (e.g., cyclodextrins), monosaccharides, disaccharides, oligosaccharides or polysaccharides.

According to certain embodiments, the pharmaceutical composition does not contain any monosaccharides or disaccharides.

According to certain embodiments, the pharmaceutical composition does not contain any disaccharides.

According to certain embodiments, the pharmaceutical composition does not contain any sugar alcohols (e.g., sorbitol).

According to certain embodiments, the pharmaceutical composition does not contain any cryoprotectants (e.g., sugar, sugar alcohol, glycerol, ethylene glycol, propylene glycol, dimethylsulfoxide, etc.) In certain embodiments, the pharmaceutical composition contains about 0.5 mg/mL of radioconjugate and conjugate intermediate and about 0.5% w/v % sodium ascorbate. In other embodiments, the pharmaceutical composition contains about 0.5 mg/mL of the radioconjugate and conjugate intermediate and about 0.04 w/v % Polysorbate 20. In further embodiments, the pharmaceutical composition contains about 0.5 mg/mL of the radioconjugate and conjugate intermediate and about 25-27 mM acetate buffer. In yet other embodiments, the pharmaceutical contains about 0.5 mg/mL of the radioconjugate and conjugate intermediate, about 0.5 w/v % sodium ascorbate and about 0.04 w/v % Polysorbate 20. In still further embodiments, the pharmaceutical contains about 0.5 mg/mL of the radioconjugate and conjugate intermediate, about 0.5 w/v % sodium ascorbate and about 25-27 mM acetate buffer. In other embodiments, the pharmaceutical contains about 0.5 mg/mL of the radioconjugate and conjugate intermediate, about 0.04 w/v % Polysorbate 20, and about 25-27 mM acetate buffer. In further embodiments, the pharmaceutical composition contains about 0.5 mg/mL of the radioconjugate and conjugate intermediate, about 0.5 w/v % sodium ascorbate about 0.04 w/v % Polysorbate 20, and about 25-27 mM acetate buffer.

In certain aspects, the pharmaceutical composition contains radioconjugate and acetate buffer. In other aspects, the pharmaceutical composition contains radioconjugate, acetate buffer and sodium ascorbate. In further aspects, the pharmaceutical composition contains radioconjugate and polysorbate 20. In yet other aspects, the pharmaceutical composition contains radioconjugate, sodium ascorbate, polysorbate 20, and acetate buffer. In still further aspects, the pharmaceutical composition contains ²²⁵Ac-DOTA-h11B6, acetate buffer. In other aspects, the pharmaceutical composition contains ²²⁵Ac-DOTA-h11B6, acetate buffer and sodium ascorbate. In further aspects, the pharmaceutical composition contains ²²⁵Ac-DOTA-h11B6, polysorbate 20. In yet other aspects, the pharmaceutical composition contains ²²⁵Ac-DOTA-h11B6, sodium ascorbate, polysorbate 20, and acetate buffer. In still further aspects, the pharmaceutical composition contains ²²⁵Ac-TOPA-h11B6, acetate buffer. In other aspects, the pharmaceutical composition contains ²²⁵Ac-TOPA-h11B6, acetate buffer and sodium ascorbate. In further aspects, the pharmaceutical composition contains ²²⁵Ac-TOPA-h11B6, polysorbate 20. In yet other aspects, the pharmaceutical composition contains ²²⁵Ac-TOPA-h11B6, sodium ascorbate, polysorbate 20, and acetate buffer.

According to an embodiment, pharmaceutical composition contains ²²⁵Ac-DOTA-h11B6 and DOTA-h11B6 in a total amount of about 0.5 mg/mL in 25-27 mM acetate (e.g., 25 mM or 26.75 mM), 0.5% sodium ascorbate, and 0.04% polysorbate 20 in sterile water, preferably at a pH of about 5.5. According to certain embodiments, the radioactive ²²⁵Ac dose of ²²⁵Ac-DOTA-h11B6 is targeted for about 50, about 100, about 150, or about 200 μCi in 4 mL (about 2 mg h11B6 mass amount) at the anticipated time of dosing. According to other embodiments, the radioactive ²²⁵Ac dose of ²²⁵Ac-DOTA-h11B6 is targeted for greater than 200 μCi at the time of dosing (e.g., about 250 μCi or about 300 μCi or about 350 μCi, etc.); for example, the dose may comprise about 250 μCi in about 8 mL or about 300 μCi in about 8 mL or about 350 μCi in about 8 mL (e.g., per about 2 mg h11B6 or about 4 mg or about 6 mg or about 8 mg or about 10 mg mass amount per dose) at the anticipated time of dosing.

According to an embodiment, pharmaceutical composition contains ²²⁵Ac-TOPA-h11B6 and TOPA-h11B6 in a total amount of about 0.5 mg/mL in 25-27 mM acetate (e.g., 25 mM or 26.75 mM), 0.5% sodium ascorbate, and 0.04% polysorbate 20 in sterile water, preferably at a pH of about 5.5. According to certain embodiments, the radioactive ¹¹⁵Ac dose of ²²⁵Ac-TOPA-h11B6 is targeted for about 50, about 100, about 150, or about 200 μCi in 4 mL (about 2 mg h11B6 mass amount) at the anticipated time of dosing. According to other embodiments, the radioactive ²²⁵Ac dose of ²²⁵Ac-TOPA-h11B6 is targeted for greater than 200 μCi at the time of dosing (e.g., about 250 μCi or about 300 μCi or about 350 μCi, etc.); for example, the dose may comprise about 250 μCi in about 8 mL or about 300 μCi in about 8 mL or about 350 μCi in about 8 mL (e.g., per about 2 mg h11B6 or about 4 mg or about 6 mg or about 8 mg or about 10 mg mass amount per dose) at the anticipated time of dosing.

### Enumerated Embodiments

Provided below are numbered exemplary embodiments of the present invention.
1. A method of treating cancer in a patient, the method comprising:
   administering to the patient a therapeutically effective amount of a pharmaceutical composition comprising a radioconjugate and one or more pharmaceutically acceptable excipients, wherein:
   the radioconjugate comprises at least one radiometal complex conjugated to an antibody, or an antigen binding fragment, with binding specificity for hK2,
   the radiometal complex comprises a radiometal, and
   the radiometal provides a targeted radioactivity from about 50 μCi to about 350 μCi per dose of the pharmaceutical composition at the time of dosing.
1A. A method of treating cancer in a patient, the method comprising:
   administering to the patient a therapeutically effective amount of a pharmaceutical composition comprising a radioconjugate and one or more pharmaceutically acceptable excipients, wherein:
   the radioconjugate comprises at least one radiometal complex conjugated to an antibody, or an antigen binding fragment, with binding specificity for hK2,
   the radiometal complex comprises a radiometal, and
the radiometal provides a targeted radioactivity from about 350 μCi to about 500 μCi per dose of the pharmaceutical composition at the time of dosing
2. The method according to embodiment 1 or 1A, wherein the radioconjugate comprises at least one radiometal complex conjugated to an antibody with binding specificity for hK2.
3. The method according to embodiment 2, wherein the antibody comprises a heavy chain variable region comprising the amino acid sequences of SEQ ID NO:1 and SEQ ID NO:2 and SEQ ID NO:3; and a light chain variable region comprising the amino acid sequences of SEQ ID NO:4 and SEQ ID NO:5 and SEQ ID NO:6.
4. The method according to embodiment 2 or 3, wherein the antibody comprises a heavy chain variable region (VH) having at least 80%, at least 85%, at least 90%, at least 95%, or at least 98% sequence identity to the amino acid sequence of SEQ ID NO: 8, and a light chain variable region (VL) having at least 80%, at least 85%, at least 90%, at least 95%, or at least 98% sequence identity to the amino acid sequence of SEQ ID NO: 9.
5. The method according to embodiment 2 or 3, wherein the antibody comprises a heavy chain variable region (VH) comprising the amino acid sequence of SEQ ID NO: 8, and a light chain variable region (VL) comprising the amino acid sequence of SEQ ID NO: 9.
6. The method according to any of embodiments 2-5, wherein the antibody comprises a heavy chain constant region having at least 80%, at least 85%, at least 90%, at least 95%, or at least 98% sequence identity to the amino acid sequence of SEQ ID NO: 10, and a light chain constant region having at least 80%, at least 85%, at least 90%, at least 95%, or at least 98% sequence identity to the amino acid sequence of SEQ ID NO: 11.
7. The method according to any of embodiments 2-5, wherein the antibody comprises a heavy chain constant region comprising the amino acid sequence of SEQ ID NO: 10, and a light chain constant region comprising the amino acid sequence of SEQ ID NO: 11.
8. The method according to any of embodiments 2-7, wherein the antibody comprises a heavy chain having at least 80%, at least 85%, at least 90%, at least 95%, or at least 98% sequence identity to the amino acid sequence of SEQ ID NO: 12, and a light chain having at least 80%, at least 85%, at least 90%, at least 95%, or at least 98% sequence identity to the amino acid sequence of SEQ ID NO: 13.
9. The method according to any of embodiments 2-7, wherein the antibody comprises a heavy chain having the amino acid sequence of SEQ ID NO: 12, and a light chain having the amino acid sequence of SEQ ID NO: 13.
10. The method according to any of embodiments 1-9 or 1A, wherein the radiometal is selected from the group consisting of ²²⁵Ac, ¹¹¹In, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th.
11. The method according to any of embodiments 1-9 or 1A, wherein the radiometal is ²²⁵Ac.
12. The method according to any of embodiments 1-11 or 1A, wherein the radiometal complex comprises a chelator that is selected from the group consisting of 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (D03A), and derivatives thereof.
13. The method according to any of embodiments 1-11 or 1A, wherein the radiometal complex comprises a chelator that is DOTA.
   13A. The method according to any of embodiments 1-11 or 1A, wherein the radiometal complex comprises a chelator that is TOPA.
14. The method according to any of embodiments 1-13 or 1A, wherein the radiometal complex comprises ²²⁵Ac chelated to DOTA.
14A. The method according to any of embodiments 1-11 or 1A, wherein the radiometal complex comprises ²²⁵Ac chelated to TOPA.
15. The method according to any of embodiments 1-11 or 1A, wherein the radioconjugate comprises the radiometal chelated to (a) a compound of formula (IV)

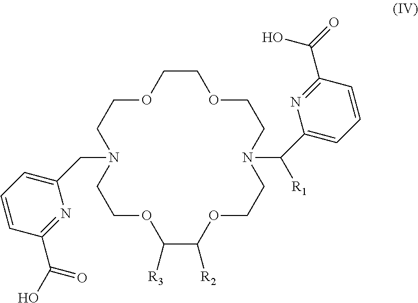

or a pharmaceutically acceptable salt thereof, wherein:
   R₁ is hydrogen and R₂ is -L₁-R₄;
   alternatively, R₁ is -L₁-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L₁-R₄,
   L₁ is absent or a linker; and
   R₄ is the antibody; or
(b) a compound of formula (V)

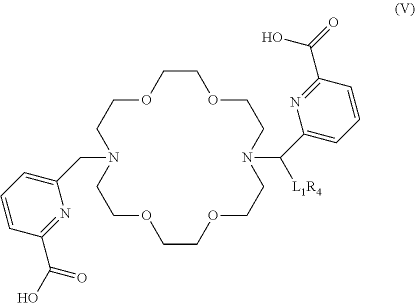

or a pharmaceutically acceptable salt thereof, wherein:
   L₁ is absent or a linker; and
   R₄ is the antibody;
   for example, wherein the chelator used to form the radioconjugate is a compound of the following formula or a pharmaceutically acceptable salt thereof:

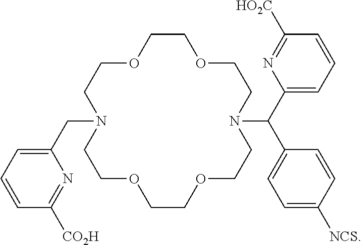

16. The method according to any of embodiments 2-15 or 13A or 14A, wherein the radiometal is ²²⁵Ac and the radiometal provides a targeted specific activity from about 25 μCi to about 350 μCi per about 2 mg of total antibody, or from about 50 μCi to about 350 μCi per about 2 mg of total antibody.
16A. The method according to any of embodiments 2-15 or 13A or 14A, wherein the radiometal is ²²⁵Ac and the radiometal provides a targeted specific activity from about 25 μCi to about 350 μCi per from about 2 mg of total antibody to about 10 mg total antibody (e.g., per about 4 mg of total antibody); or from about 50 μCi to about 350 μCi per from about 2 mg of total antibody to about 10 mg total antibody (e.g., per about 4 mg of total antibody).
17. The method according to any of embodiments 2-15 or 13A or 14A, wherein the radiometal is ²²⁵Ac and the radiometal provides a targeted specific activity from about 25 μCi to about 300 μCi per about 2 mg of total antibody, or from about 50 μCi to about 300 μCi per about 2 mg of total antibody.
   17A. The method according to any of embodiments 2-15 or 13A or 14A, wherein the radiometal is ²²⁵Ac and the radiometal provides a targeted specific activity from about 25 μCi to about 300 μCi per from about 2 mg of total antibody to about 10 mg total antibody (e.g., per about 4 mg of total antibody); or from about 50 μCi to about 300 μCi per from about 2 mg of total antibody to about 10 mg total antibody (e.g., per about 4 mg of total antibody).
18. The method according to any of embodiments 2-15 or 13A or 14A, wherein the radiometal is ²²⁵Ac and the radiometal provides a targeted specific activity from about 25 μCi to about 250 μCi per about 2 mg of total antibody, or from about 50 μCi to about 250 μCi per about 2 mg of total antibody.
   18A. The method according to any of embodiments 2-15 or 13A or 14A, wherein the radiometal is ²²⁵Ac and the radiometal provides a targeted specific activity from about 25 μCi to about 250 μCi per from about 2 mg of total antibody to about 10 mg total antibody (e.g., per about 4 mg of total antibody); or from about 50 μCi to about 250 μCi per from about 2 mg of total antibody to about 10 mg total antibody (e.g., per about 4 mg of total antibody).
19. The method according to any of embodiments 2-15 or 13A or 14A, wherein the radiometal is ²²⁵Ac and the radiometal provides a targeted specific activity from about 25 μCi to about 200 μCi per about 2 mg of total antibody, or from about 50 μCi to about 200 μCi per about 2 mg of total antibody.
20. The method according to any of embodiments 2-15 or 13A or 14A, wherein the radiometal is ²²⁵Ac and the radiometal provides a targeted specific activity from about 25 μCi to about 150 μCi per about 2 mg of total antibody, or from about 50 μCi to about 150 μCi per about 2 mg of total antibody.
21. The method according to any of embodiments 2-15 or 13A or 14A, wherein the radiometal is ²²⁵Ac and the radiometal provides a targeted specific activity from about 25 μCi to about 100 μCi per about 2 mg of total antibody, or from about 50 μCi to about 100 μCi per about 2 mg of total antibody.
22. The method according to any of embodiments 2-15 or 13A or 14A, wherein the radiometal is ²²⁵Ac and the radiometal provides a targeted specific activity from about 150 μCi to about 250 μCi per about 2 mg of total antibody.
23. The method according to any of embodiments 2-15 or 13A or 14A, wherein the radiometal is ²²⁵Ac and the radiometal provides a targeted specific activity of about 50 μCi per about 2 mg of total antibody.
24. The method according to any of embodiments 2-15 or 13A or 14A, wherein the radiometal is ²²⁵Ac and the radiometal provides a targeted specific activity of about 100 μCi per about 2 mg of total antibody.
25. The method according to any of embodiments 2-15 or 13A or 14A, wherein the radiometal is ²²⁵Ac and the radiometal provides a targeted specific activity of about 150 μCi per about 2 mg of total antibody.
26. The method according to any of embodiments 2-15 or 13A or 14A, wherein the radiometal is ²²⁵Ac and the radiometal provides a targeted specific activity of about 200 ρCi per about 2 mg of total antibody.
27. The method according to any of embodiments 2-26, or 13A, or 14A, or 16A, or 17A, or 18A, wherein the pharmaceutical composition comprises a targeted radioactive concentration of from about 1 μCi/mL to about 100 μCi/mL, or from about 5 μCi/mL to about 75 μCi/mL, or from about 10 μCi/mL to about 60 μCi/mL, or from about 12.5 μCi/mL to about 50 μCi/mL, or about 12.5 μCi/mL, or about 25 μCi/mL, or about 37.5 μCi/mL, or about 50 μCi/mL.
28. The method according to any of embodiments 2-26, or 13A, or 14A, or 16A, or 17A, or 18A, wherein the pharmaceutical composition comprises from about 1 mg to about 5 mg of total antibody, or from about 1 mg to about 4 mg of total antibody.
   28A. The method according to any of embodiments 2-26, or 13A, or 14A, or 16A, or 17A, or 18A, wherein the pharmaceutical composition comprises from about 1 mg to about 10 mg of total antibody, or from about 2 mg to about 8 mg of total antibody.
29. The method according to any of embodiments 2-26, or 13A, or 14A, or 16A, or 17A, or 18A, wherein the pharmaceutical composition comprises from about 1 mg to about 4 mg of total antibody.
30. The method according to any of embodiments 2-26, or 13A, or 14A, or 16A, or 17A, or 18A, wherein the pharmaceutical composition comprises from about 1 mg to about 3 mg of total antibody.
31. The method according to any of embodiments 2-26, or 13A, or 14A, or 16A, or 17A, or 18A, wherein the pharmaceutical composition comprises from about 1.5 to about 2.5 mg of total antibody.
32. The method according to any of embodiments 2-26, or 13A, or 14A, or 16A, or 17A, or 18A, wherein the pharmaceutical composition comprises about 2 mg of total antibody.
   32A. The method according to any of embodiments 2-26, or 13A, or 14A, or 16A, or 17A, or 18A, wherein the pharmaceutical composition comprises about 4 mg of total antibody or about 8 mg of total antibody
33. The method according to any of embodiments 1-31, or 1A, or 13A, or 14A, or 16A, or 17A, or 18A, or 28A, wherein the one or more pharmaceutically acceptable excipients comprise one or more radioprotectants.
34. The method according to embodiment 32 or 32A, wherein the one or more radioprotectants comprise sodium ascorbate, gentisic acid, or a combination thereof.
35. The method according to embodiment 32 or 32A, wherein the one or more radioprotectants comprise sodium ascorbate.
36. The method according to embodiment 32 or 32A, wherein the one or more radioprotectants comprise gentisic acid.
37. The method according to any of embodiments 1-35, or 1A, or 13A, or 14A, or 16A, or 17A, or 18A, or 28A, or 32A, wherein the one or more pharmaceutically acceptable excipients further comprise one or more surfactants.
38. The method according to embodiment 36, wherein the one or more surfactants comprise polysorbate 20.
39. The method according to any of embodiments 1-37, or 1A, or 13A, or 14A, or 16A, or 17A, or 18A, or 28A, or 32A, wherein the one or more pharmaceutically acceptable excipients further comprise an acetate buffer.
40. The method according to any of embodiments 1-38, or 1A, or 13A, or 14A, or 16A, or 17A, or 18A, or 28A, or 32A, wherein the pharmaceutical composition comprises the radioconjugate, sodium ascorbate, polysorbate 20, acetate buffer and water (and acetic acid may optionally be added for pH adjustment).
41. The method according to any of embodiments 1-38, or 1A, or 13A, or 14A, or 16A, or 17A, or 18A, or 28A, or 32A, wherein the pharmaceutical composition comprises the radioconjugate, about 24-28 mM acetate, about 0.25-0.75% sodium ascorbate, and about 0.01-0.15% polysorbate 20 in water.
42. The method according to any of embodiments 1-38, or 1A, or 13A, or 14A, or 16A, or 17A, or 18A, or 28A, or 32A, wherein the pharmaceutical composition comprises the radioconjugate, about 25 mM acetate, about 0.5% sodium ascorbate, and about 0.04% polysorbate 20 in water.
43. The method according to any of embodiments 1-38, or 1A, or 13A, or 14A, or 16A, or 17A, or 18A, or 28A, or 32A, wherein the pharmaceutical composition comprises the radioconjugate, about 26.75 mM acetate, about 0.5% sodium ascorbate, and about 0.04% polysorbate 20 in water.
44. The method according to any of embodiments 1-42, or 1A, or 13A, or 14A, or 16A, or 17A, or 18A, or 28A, or 32A, wherein the pharmaceutical composition has a pH from about 5 to about 6 (e.g., about 5.5).
45. The method according to any of embodiments 1-43, or 1A, or 13A, or 14A, or 16A, or 17A, or 18A, or 28A, or 32A, wherein the pharmaceutical composition does not contain any preservatives.
46. The method according to any of embodiments 1-44, or 1A, or 13A, or 14A, or 16A, or 17A, or 18A, or 28A, or 32A, wherein the pharmaceutical composition does not contain any sucrose.
47. The method according to any of embodiments 1-44, or 1A, or 13A, or 14A, or 16A, or 17A, or 18A, or 28A, or 32A, wherein the pharmaceutical composition does not contain any monosaccharides, disaccharides, oligosaccharides or polysaccharides.
48. The method according to any of embodiments 1-44, or 1A, or 13A, or 14A, or 16A, or 17A, or 18A, or 28A, or 32A, wherein the pharmaceutical composition does not contain any monosaccharides or disaccharides.
49. The method according to any of embodiments 1-44, or 1A, or 13A, or 14A, or 16A, or 17A, or 18A, or 28A, or 32A, wherein the pharmaceutical composition does not contain any disaccharides.
50. The method according to any of embodiments 1-48, or 1A, or 13A, or 14A, or 16A, or 17A, or 18A, or 28A, or 32A, wherein the pharmaceutical composition is stable at a temperature range of about 2-8° C. for at least about 72 hours, or for at least about 96 hours, or at least about 120 hours.
51. The method according to any of embodiments 2-49, or 13A, or 14A, or 16A, or 17A, or 18A, or 28A, or 32A, wherein the dose of the pharmaceutical composition has a volume from about 1 mL to about 20 mL, or about 1 mL to about 10 mL, or about 2 mL to about 6 mL, or about 3 mL to about 5 mL, or about 4 mL.
52. The method according to any of embodiments 2-49, or 13A, or 14A, or 16A, or 17A, or 18A, or 28A, or 32A, wherein the dose of the pharmaceutical composition comprises about 2 mg of total antibody per about 4 mL of the dose.
53. The method according to any of embodiments 2-51, or 13A, or 14A, or 16A, or 17A, or 18A, or 28A, or 32A, wherein the pharmaceutical composition comprises total antibody in an amount of about 0.1-1.0 mg/mL, or about 0.4-0.6 mg/mL, or about 0.5 mg/mL.
54. The method according to any of embodiments 2-52, or 13A, or 14A, or 16A, or 17A, or 18A, or 28A, or 32A, wherein the pharmaceutical composition further comprises non-radiolabeled antibody (e.g., a conjugate intermediate, such as DOTA-mAb, e.g., DOTA-h11B6), wherein the non-radiolabeled antibody is the same antibody as the antibody conjugated to the radiometal complex.
55. The method according to embodiment 53, wherein the total amount of the conjugated antibody and the non-radiolabeled antibody does not exceed about 10 mg, or about 9 mg, or about 8 mg, or about 7 mg, or about 6 mg, or about 5 mg, or about 4 mg, or about 3 mg, or about 2 mg.
56. The method according to any of embodiments 1-54 or 1A, or 13A, or 14A, or 16A, or 17A, or 18A, or 28A, or 32A, comprising administering the pharmaceutical composition to the patient intravenously.
57. The method according to any of embodiments 1-55 or 1A, or 13A, or 14A, or 16A, or 17A, or 18A, or 28A, or 32A, comprising administering the pharmaceutical composition to the patient within about 168 hours, or within about 144 hours, or within about 120 hours, or within about 96 hours, or within about 72 hours, or within about 48 hours, or within about 24 hours from chelation of the radiometal to a conjugate intermediate to form the radioconjugate.
58. The method according to any of embodiments 1-56 or 1A, or 13A, or 14A, or 16A, or 17A, or 18A, or 28A, or 32A, comprising administering the pharmaceutical composition to the patient once every about 4 weeks.
59. The method according to any of embodiments 1-56, or 1A, or 13A, or 14A, or 16A, or 17A, or 18A, or 28A, or 32A, comprising administering the pharmaceutical composition to the patient once every about 8 weeks.
60. The method according to any of embodiments 1-56, or 1A, or 13A, or 14A, or 16A, or 17A, or 18A, or 28A, or 32A, comprising administering the pharmaceutical composition to the patient once every about 12 weeks.
61. The method according to any of embodiments 1-59, or 1A, or 13A, or 14A, or 16A, or 17A, or 18A, or 28A, or 32A, wherein the cancer is prostate cancer.
62. The method according to any of embodiments 1-59, or 1A, or 13A, or 14A, or 16A, or 17A, or 18A, or 28A, or 32A, wherein the cancer is non-localized prostate cancer.
63. The method according to any of embodiments 1-59, or 1A, or 13A, or 14A, or 16A, or 17A, or 18A, or 28A, or 32A, wherein the cancer is metastatic prostate cancer.
64. The method according to any of embodiments 1-59, or 1A, or 13A, or 14A, or 16A, or 17A, or 18A, or 28A, or 32A, wherein the cancer is castration-resistant prostate cancer (CRPC).
65. The method according to any of embodiments 1-59, or 1A, or 13A, or 14A, or 16A, or 17A, or 18A, or 28A, or 32A, wherein the cancer is metastatic castration-resistant prostate cancer (mCRPC).
66. The method according to any of embodiments 1-59, or 1A, or 13A, or 14A, or 16A, or 17A, or 18A, or 28A, or 32A, wherein the cancer is mCRPC with adenocarcinoma.
67. The method according to any of embodiments 1-65, or 1A, or 13A, or 14A, or 16A, or 17A, or 18A, or 28A, or 32A, wherein testosterone castrate levels of the patient are about 50 ng/dL or less.
68. The method according to any of embodiments 1-66, or 1A, or 13A, or 14A, or 16A, or 17A, or 18A, or 28A, or 32A, wherein the patient had prior exposure to at least one androgen receptor (AR) targeted therapy.
69. The method according to embodiment 67, wherein the AR targeted therapy is abiraterone acetate, enzalutamide, apalutamide, darolutamide, or combinations of any of the foregoing.
70. The method according to any of embodiments 1-68, or 1A, or 13A, or 14A, or 16A, or 17A, or 18A, or 28A, or 32A, wherein the patient had prior chemotherapy.
71. The method according to embodiment 69, wherein the chemotherapy involved administration of taxane.
72. The method according to any of embodiments 1-70, or 1A, or 13A, or 14A, or 16A, or 17A, or 18A, or 28A, or 32A, wherein the patient had prior orchiectomy or medical castration.
73. The method according to any of embodiments 1-71, or 1A, or 13A, or 14A, or 16A, or 17A, or 18A, or 28A, or 32A, wherein the patient is receiving ongoing androgen deprivation therapy with a gonadotropin releasing hormone (GnRH) agonist or antagonist.
74. The method according to any of embodiments 1-72 or 1A, or 13A, or 14A, or 16A, or 17A, or 18A, or 28A, or 32A, comprising administering the dose in a single administration to the patient.
75. The method according to any of embodiments 1-72 or 1A, or 13A, or 14A, or 16A, or 17A, or 18A, or 28A, or 32A, comprising administering the dose in multiple administrations of more than one sub-dose.
   75A. The method according to embodiment 75, comprising administering the dose in two sub-doses (e.g., two 4-mL sub-doses).
76. A pharmaceutical composition comprising:
   a radioconjugate and one or more pharmaceutically acceptable excipients, wherein:
   the radioconjugate comprises at least one radiometal complex conjugated to an antibody, or an antigen binding fragment, with binding specificity for hK2, and
   the radiometal complex comprises a radiometal.
77. The pharmaceutical composition according to embodiment 76, wherein the one or more pharmaceutically acceptable excipients comprise one or more radioprotectants.
78. The pharmaceutical composition according to embodiment 76 or 77, wherein the radioconjugate comprises at least one radiometal complex conjugated to an antibody with binding specificity for hK2.
79. The pharmaceutical composition according to embodiment 78, wherein the antibody comprises a heavy chain variable region comprising the amino acid sequences of SEQ ID NO:1 and SEQ ID NO:2 and SEQ ID NO:3; and a light chain variable region comprising the amino acid sequences of SEQ ID NO:4 and SEQ ID NO:5 and SEQ ID NO:6.
80. The pharmaceutical composition according to embodiment 78 or embodiment 79, wherein the antibody comprises a heavy chain variable region (VH) having at least 80%, at least 85%, at least 90%, at least 95%, or at least 98% sequence identity to the amino acid sequence of SEQ ID NO: 8, and a light chain variable region (VL) having at least 80%, at least 85%, at least 90%, at least 95%, or at least 98% sequence identity to the amino acid sequence of SEQ ID NO: 9.
81. The pharmaceutical composition according to embodiment 78 or embodiment 79, wherein the antibody comprises a heavy chain variable region (VH) comprising the amino acid sequence of SEQ ID NO: 8, and a light chain variable region (VL) comprising the amino acid sequence of SEQ ID NO: 9.
82. The pharmaceutical composition according to any of embodiments 78-81, wherein the antibody comprises a heavy chain constant region having at least 80%, at least 85%, at least 90%, at least 95%, or at least 98% sequence identity to the amino acid sequence of SEQ ID NO: 10, and a light chain constant region having at least 80%, at least 85%, at least 90%, at least 95%, or at least 98% sequence identity to the amino acid sequence of SEQ ID NO: 11.
83. The pharmaceutical composition according to any of embodiments 78-81, wherein the antibody comprises a heavy chain constant region comprising the amino acid sequence of SEQ ID NO: 10, and a light chain constant region comprising the amino acid sequence of SEQ ID NO: 11.
84. The pharmaceutical composition according to any of embodiments 78-83, wherein the antibody comprises a heavy chain having at least 80%, at least 85%, at least 90%, at least 95%, or at least 98% sequence identity to the amino acid sequence of SEQ ID NO: 12, and a light chain having at least 80%, at least 85%, at least 90%, at least 95%, or at least 98% sequence identity to the amino acid sequence of SEQ ID NO: 13.
85. The pharmaceutical composition according to any of embodiments 78-83, wherein the antibody comprises a heavy chain having the amino acid sequence of SEQ ID NO: 12, and a light chain having the amino acid sequence of SEQ ID NO: 13.
86. The pharmaceutical composition according to any of embodiments 76-85, wherein the radiometal is selected from the group consisting of ²²⁵Ac, ¹¹¹In, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir ¹⁹⁸Au, ¹⁹⁹Au, ²¹¹At, ²¹²Pb, ²¹²Bi, ²¹³Bi, ²²³Ra, ²⁵⁵Fm and ²²⁷Th.
87. The pharmaceutical composition according to any of embodiments 76-85, wherein the radiometal is ²²⁵Ac.
88. The pharmaceutical composition according to any of embodiments 76-87, wherein the radiometal complex comprises a chelator that is selected from the group consisting of 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), and derivatives thereof.
89. The pharmaceutical composition according to any of embodiments 76-87, wherein the radiometal complex comprises a chelator that is DOTA.
90. The pharmaceutical composition according to any of embodiments 76-89, wherein the radiometal complex comprises ²¹⁵Ac chelated to DOTA.
91. The pharmaceutical composition according to any of embodiments 76-87, wherein the radioconjugate comprises the radiometal chelated to (a) a compound of formula (IV)

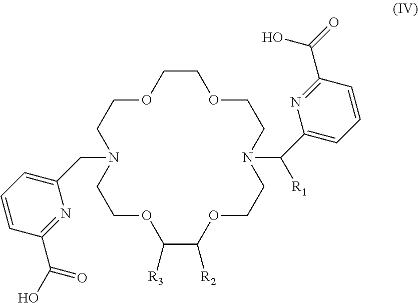

or a pharmaceutically acceptable salt thereof, wherein:
   R₁ is hydrogen and R₂ is -L₁-R₄;
   alternatively, R₁ is -L₁-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L₁-R₄,
   L₁ is absent or a linker; and
   R₄ is the antibody; or
(b) a compound of formula (V)

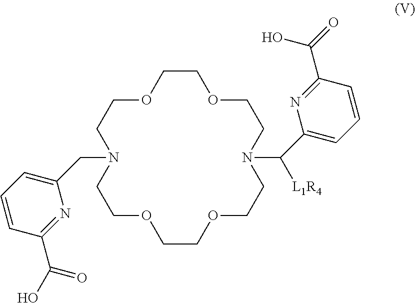

or a pharmaceutically acceptable salt thereof, wherein:
   L₁ is absent or a linker; and
   R₄ is the antibody;
for example, wherein the chelator used to form the radioconjugate is a compound of the following formula or a pharmaceutically acceptable salt thereof:

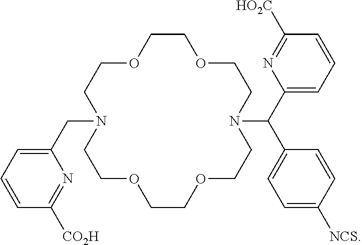

92. The pharmaceutical composition according to any of embodiments 77-91, wherein the one or more radioprotectants comprise sodium ascorbate, gentisic acid, or a combination thereof (e.g., in an amount of about 0.1 to about 5 w/v %, or about 0.1 to about 4 w/v %, or about 0.1 to about 3 w/v %, about 0.1 to about 2 w/v %, or about 0.1 to about 1 w/v %, or about 0.25 to about 0.75 w/v %, or about 0.5 w/v %).
93. The pharmaceutical composition according to any of embodiments 77-91, wherein the one or more radioprotectants comprise sodium ascorbate (e.g., in an amount of about 0.1 to about 5 w/v %, or about 0.1 to about 4 w/v %, or about 0.1 to about 3 w/v %, about 0.1 to about 2 w/v %, or about 0.1 to about 1 w/v %, or about 0.25 to about 0.75 w/v %, or about 0.5 w/v %).
94. The pharmaceutical composition according to any of embodiments 77-91, wherein the one or more radioprotectants comprise gentisic acid (e.g., in an amount of about 0.1 to about 5 w/v %, or about 0.1 to about 4 w/v %, or about 0.1 to about 3 w/v %, about 0.1 to about 2 w/v %, or about 0.1 to about 1 w/v %, or about 0.25 to about 0.75 w/v %, or about 0.5 w/v %).
95. The pharmaceutical composition according to any of embodiments 76-94, wherein the one or more pharmaceutically acceptable excipients further comprise one or more surfactants.
96. The pharmaceutical composition according to embodiment 95, wherein the one or more surfactants comprise polysorbate 20.
97. The pharmaceutical composition according to any of embodiments 76-96, wherein the one or more pharmaceutically acceptable excipients further comprise an acetate buffer.
98. The pharmaceutical composition according to any of embodiments 76-97 comprising the radioconjugate, sodium ascorbate, polysorbate 20, acetate buffer and water (and acetic acid may optionally be added for pH adjustment).
99. The pharmaceutical composition according to any of embodiments 76-97 comprising the radioconjugate, about 24-28 mM acetate, about 0.25-0.75 w/v % sodium ascorbate, and about 0.01-0.15 w/v % polysorbate 20 in water.
100. The pharmaceutical composition according to any of embodiments 76-97 comprising the radioconjugate, about 25 mM acetate, about 0.5 w/v % sodium ascorbate, and about 0.04 w/v % polysorbate 20 in water.
101. The pharmaceutical composition according to any of embodiments 76-97 comprising the radioconjugate, about 26.75 mM acetate, about 0.5 w/v % sodium ascorbate, and about 0.04 w/v % polysorbate 20 in water.
102. The pharmaceutical composition according to any of embodiments 76-101, wherein the pharmaceutical composition has a pH from about 5 to about 6 (e.g., about 5.5).
103. The pharmaceutical composition according to any of embodiments 76-101, wherein the pharmaceutical composition does not contain any preservatives.
104. The pharmaceutical composition according to any of embodiments 76-103, wherein the pharmaceutical composition does not contain any sucrose.
105. The pharmaceutical composition according to any of embodiments 76-103, wherein the pharmaceutical composition does not contain any monosaccharides, disaccharides, oligosaccharides or polysaccharides.
106. The pharmaceutical composition according to any of embodiments 76-103, wherein the pharmaceutical composition does not contain any monosaccharides or disaccharides.
107. The pharmaceutical composition according to any of embodiments 76-103, wherein the pharmaceutical composition does not contain any disaccharides.
108. The pharmaceutical composition according to any of embodiments 76-103, wherein the one or more pharmaceutically acceptable excipients consist of, or consist essentially of, acetate buffer, sodium ascorbate and polysorbate 20 in water.
109. The pharmaceutical composition according to any of embodiments 76-108, wherein the pharmaceutical composition is formulated for intravenous administration.
110. The pharmaceutical composition according to any of embodiments 76-109, wherein the pharmaceutical composition is stable at a temperature range of about 2-8° C. for at least 72 hours, or at least 96 hours, or at least 120 hours.
111. The pharmaceutical composition according to any of embodiments 77-110, wherein the radioconjugate comprises an average of from about 1 to about 4, or about 2 to about 3 chelator molecules conjugated to the antibody.
112. The pharmaceutical composition according to any of embodiments 77-111, wherein the radiometal is ²²⁵Ac and the radiometal provides a specific activity from about 50 μCi to about 350 μCi per about 2 mg of total antibody.
   112A. The pharmaceutical composition according to any of embodiments 77-111, wherein the radiometal is ²²⁵Ac and the radiometal provides a specific activity from about 50 μCi to about 350 μCi per from about 2 mg to about 10 mg of total antibody
   112B. The pharmaceutical composition according to any of embodiments 77-111, wherein the radiometal is ²²⁵Ac and the radiometal provides a specific activity from about 350 μCi to about 500 μCi per from about 2 mg to about 10 mg of total antibody.
113. The pharmaceutical composition according to any of embodiments 77-111, wherein the radiometal is ²²⁵Ac and the radiometal provides a specific activity from about 50 μCi to about 300 μCi per about 2 mg of total antibody.
   113A. The pharmaceutical composition according to any of embodiments 77-111, wherein the radiometal is ²²⁵Ac and the radiometal provides a specific activity from about 50 μCi to about 300 μCi per from about 2 mg to about 10 mg of total antibody.
114. The pharmaceutical composition according to any of embodiments 77-111, wherein the radiometal is ²²⁵Ac and the radiometal provides a specific activity from about 50 μCi to about 250 μCi per about 2 mg of total antibody.
   114A. The pharmaceutical composition according to any of embodiments 77-111, wherein the radiometal is ²²⁵Ac and the radiometal provides a specific activity from about 50 μCi to about 250 μCi per from about 2 mg to about 10 mg of total antibody.
115. The pharmaceutical composition according to any of embodiments 77-111, wherein the radiometal is ²²⁵Ac and the radiometal provides a specific activity from about 50 μCi to about 200 μCi per about 2 mg of total antibody.
116. The pharmaceutical composition according to any of embodiments 77-111, wherein the radiometal is ²²⁵Ac and the radiometal provides a specific activity from about 50 μCi to about 150 μCi per about 2 mg of total antibody.
117. The pharmaceutical composition according to any of embodiments 77-111, wherein the radiometal is ²²⁵Ac and the radiometal provides a specific activity from about 50 μCi to about 100 μCi per about 2 mg of total antibody.
118. The pharmaceutical composition according to any of embodiments 77-111, wherein the radiometal is ²²⁵Ac and the radiometal provides a targeted specific activity from about 50 μCi to about 200 μCi per about 2 mg of total antibody at the time of dosing.
119. The pharmaceutical composition according to any of embodiments 77-111, wherein the radiometal is ²²⁵Ac and the radiometal provides a targeted specific activity of about 50 μCi per about 2 mg of total antibody at the time of dosing.
120. The pharmaceutical composition according to any of embodiments 77-111, wherein the radiometal is ²²⁵Ac and the radiometal provides a targeted specific activity of about 100 μCi per about 2 mg of total antibody at the time of dosing.
121. The pharmaceutical composition according to any of embodiments 77-111, wherein the radiometal is ²²⁵Ac and the radiometal provides a targeted specific activity of about 150 μCi per about 2 mg of total antibody at the time of dosing.
122. The pharmaceutical composition according to any of embodiments 77-111, wherein the radiometal is ²²⁵Ac and the radiometal provides a targeted specific activity of about 200 μCi per about 2 mg of total antibody at the time of dosing.
   122A. The pharmaceutical composition according to any of embodiments 77-111, wherein the radiometal is ²²⁵Ac and the radiometal provides a targeted specific activity of about 300 μCi per about 4 mg of total antibody at the time of dosing.
123. The pharmaceutical composition according to any of embodiments 77-122 comprising from about 1 mg to about 20 mg of total antibody.
124. The pharmaceutical composition according to any of embodiments 77-122 comprising from about 1 mg to about 10 mg of total antibody.
125. The pharmaceutical composition according to any of embodiments 77-122 comprising from about 1 mg to about 5 mg of total antibody.
126. The pharmaceutical composition according to any of embodiments 77-122 comprising about 2 mg of total antibody.
127. The pharmaceutical composition according to any of embodiments 77-122 comprising about 10 mg of total antibody.
128. The pharmaceutical composition according to any of embodiments 77-127 comprising a total amount of conjugate intermediate and the radioconjugate in an amount of about 0.1-1.0 mg/mL.
129. The pharmaceutical composition according to any of embodiments 77-127 comprising a total amount of conjugate intermediate and the radioconjugate in an amount of about 0.4-0.6 mg/mL.
130. The pharmaceutical composition according to any of embodiments 77-127 comprising a total amount of conjugate intermediate and the radioconjugate in an amount of about 0.5 mg/mL.
131. The pharmaceutical composition according to any of embodiments 77-127 further comprising non-radiolabeled antibody (e.g., a conjugate intermediate, such as DOTA-mAb, e.g., DOTA-h11B6), wherein the non-radiolabeled antibody is the same antibody as the antibody conjugated to the radiometal complex.
132. The pharmaceutical composition according to embodiment 131, wherein the total amount of the conjugated antibody and the non-radiolabeled antibody does not exceed about 10 mg, or about 9 mg, or about 8 mg, or about 7 mg, or about 6 mg, or about 5 mg, or about 4 mg, or about 3 mg, or about 2 mg.
133. A method for treating cancer in a patient, the method comprising administering to the patient a therapeutically effective amount of the pharmaceutical composition of any of embodiments 76-132, or 112A, or 112B, or 113A, or 114A, or 122A.
134. The method according to embodiment 133, comprising administering the pharmaceutical composition to the patient once every about 4 weeks.
135. The method according to embodiment 133, comprising administering the pharmaceutical composition to the patient once every about 8 weeks.
136. The method according to embodiment 133, comprising administering the pharmaceutical composition to the patient once every about 12 weeks.
137. The method according to any of embodiments 133-136, wherein the cancer is prostate cancer.
138. The method according to any of embodiments 133-136, wherein the cancer is non-localized prostate cancer.
139. The method according to any of embodiments 133-136, wherein the cancer is metastatic prostate cancer.
140. The method according to any of embodiments 133-136, wherein the cancer is castration-resistant prostate cancer (CRPC).
141. The method according to any of embodiments 133-136, wherein the cancer is metastatic castration-resistant prostate cancer (mCRPC).
142. The method according to any of embodiments 133-136, wherein the cancer is mCRPC with adenocarcinoma.
143. The method according to any of embodiments 133-142, wherein testosterone castrate levels of the patient are about 50 ng/dL or less.
144. The method according to any of embodiments 133-143, wherein the patient had prior exposure to at least one androgen receptor (AR) targeted therapy.
145. The method according to embodiment 144, wherein the AR targeted therapy is abiraterone acetate, enzalutamide, apalutamide, darolutamide, or combinations of any of the foregoing.
146. The method according to any of embodiments 133-145, wherein the patient had prior chemotherapy.
147. The method according to embodiment 146, wherein the chemotherapy involved administration of taxane.
148. The method according to any of embodiments 133-147, wherein the patient had prior orchiectomy or medical castration.
149. The method according to any of embodiments 133-148 wherein the patient is receiving ongoing androgen deprivation therapy with a gonadotropin releasing hormone (GnRH) agonist or antagonist.
150. A pharmaceutical composition according to any of embodiments 76-132, or 112A, or 112B, or 113A, or 114A, or 122A for use in the treatment of cancer; for example, prostate cancer, such as mCRPC.
151. A method of making the pharmaceutical composition according to any of embodiments 76-132, or 112A, or 112B, or 113A, or 114A, or 122A, the method comprising combining a first intermediate composition and a second intermediate composition to form the pharmaceutical composition, wherein: the first intermediate composition comprises the radioconjugate, and the second intermediate composition comprises a conjugate intermediate and does not contain any radioconjugate.
152. The method according to embodiment 151, wherein the radioconjugate and the conjugate intermediate comprise the same antibody.
153. The method according to embodiment 151, wherein the radioconjugate and the conjugate intermediate comprise the same antibody and the same chelator.
154. The method according to any of embodiments 151-153, wherein the first intermediate composition and the second intermediate composition comprise the same pharmaceutically acceptable excipients.
155. The method according to any of embodiments 151-153, wherein the first intermediate composition and the second intermediate composition comprise the same pharmaceutically acceptable excipients in the same amounts or substantially the same amounts.
156. The method according to any of embodiments 151-155 further comprising chelating the radiometal to a conjugate intermediate to form the radioconjugate.

According to particular embodiments, including any of enumerated embodiments 1-156, or 16A, 17A, 18A, 28A, 32A, 75A, 112A, 112B, 113A, 114A, or 122A described above, the radioconjugate is ²²⁵Ac-DOTA-h11B6.

According to particular embodiments, including any of enumerated embodiments 1-156, or 16A, 17A, 18A, 28A, 32A, 75A, 112A, 112B, 113A, 114A, or 122A described above, the radioconjugate is a TOPA-[C7]-phenylthiourea-h11B6 antibody conjugate such as ²²⁵Ac-TOPA-h11B6 (e.g., as illustrated in FIGS. 6A-6C).

The following examples are intended to further illustrate the nature of the invention. It should be understood that the following examples do not limit the present invention.

### EXAMPLES

The h11B6 antibody employed in the examples below comprises a heavy chain according to SEQ ID NO:12 and a light chain according to SEQ ID NO:13.

### Example 1: Phase 0 Imaging Study of ¹¹¹In-DOTA-h11B6 in Humans

A first-in-human Phase 0 imaging study of ¹¹¹In-DOTA-h11B6 was conducted to determine the radio-immunotherapeutic potential of targeting hK2 in subjects with advanced prostate cancer. (Clinical Trial Identifier NCT04116164).

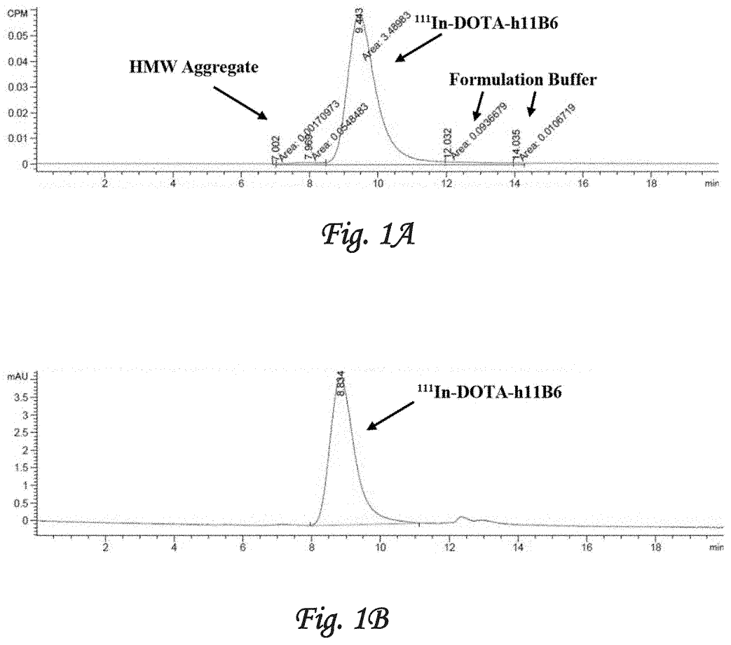

A single slow bolus infusion of 2 mg [111In]-DOTA-h11B6 (nominally 185 MBq [111In]), was administered intravenously with or without 8 mg h11B6. The formulation administered to patients was 0.5 mg/mL ¹¹¹In-DOTA-h11B6 in 25 mM acetate, 8.5% sucrose (w/v), 0.04% Polysorbate 20 (w/v), pH 5.5. UV and Radio-HPLC chromatograms of this formulation were obtained. See, FIGS. 1A and 1B.

Patients were observed for adverse events (AE) for at least 2 weeks. Serial gamma camera imaging including at least one SPECT/CT scan was performed up to 8 days post-administration. Serial blood samples were obtained over 2 weeks to determine serum radioactivity and h11B6 protein levels. Dosimetry for normal organs was estimated using OLINDA-EXM.

Results for the first 6 patients are summarized in Table 1. Treatment was tolerated in all patients with no adverse events and no evidence of enhanced accumulation in any organ including salivary glands. Initial volume of distribution appeared confined to the vascular compartment. Slow clearance of radioactivity from the vascular compartment was observed with gradual targeting to skeletal and non-skeletal lesions in all patients. The h11B6 mAb localized to bone and soft tissue metastases, has no significant normal tissue uptake, and spared salivary glands. Both serum pharmacokinetics and critical normal organ (liver, spleen, kidney) biodistribution revealed essentially no difference in biologic behavior of the antibody at the 2 mg and 10 mg antibody mass amounts.

TABLE 1
Patient characteristics, amount administered, and tumor targeting.

| Pt. No. | PSA | Tumor location | mAb Mass | [111In] (MBq) | Targeting |
|---|---|---|---|---|---|
| 1 | 19.73 | Bone | 2 mg | 218 | Yes |
| 2 | 22.58 | Liver | 2 mg | 221 | Yes |
| 3 | 39.33 | Bone | 2 mg | 202 | Yes |
| 4 | 4.96 | Bone | 10 mg | 206 | Yes |
| 5 | 49.39 | Bone | 10 mg | 196 | Yes |
| 6 | N/A | Nodes | 10 mg | 193 | Yes |

### Example 2: Preparation of Formulation “A” Comprising ²²⁵Ac-DOTA-h11B6

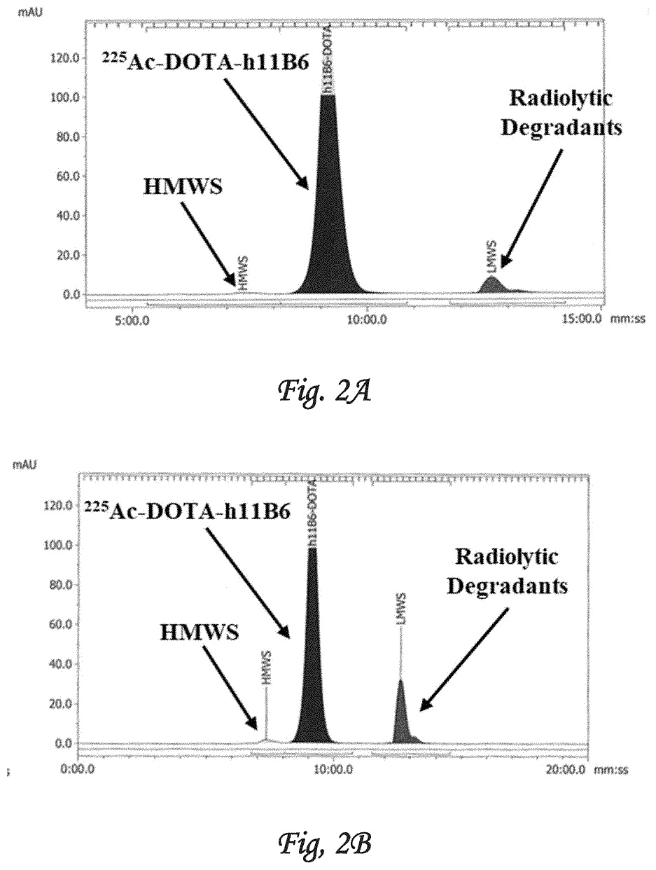

To prepare a formulation comprising actinium conjugated to h11B6, the same formulation used in Phase 0 was made, but ¹¹¹In-DOTA-h11B6 was replaced with ²²⁵Ac-DOTA-h11B6. The “Formulation A” was prepared containing 0.5 mg/mL ²²⁵Ac-DOTA-h11B6 in 26.75 mM acetate, 8.5% sucrose (w/v), and 0.04% Polysorbate 20 (w/v), pH 5.5. Radiolytic degradants were observed in UV and Radio-HPLC chromatograms of this formulation. See, FIGS. 2A and 2B. The radiolytic degradants were identified as arising from radiolysis of sucrose in the formulation.

### Example 3: Preparation of a Formulation “B” Comprising ²²⁵Ac-DOTA-h11B6

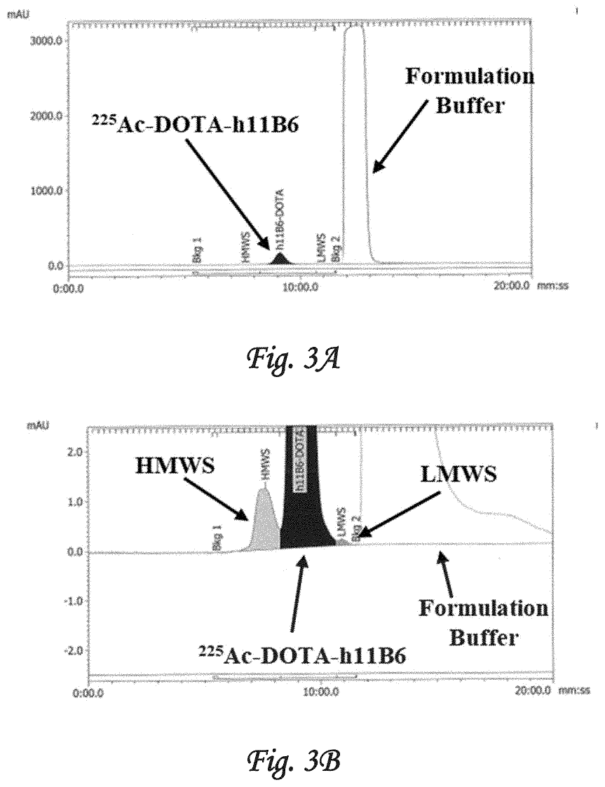

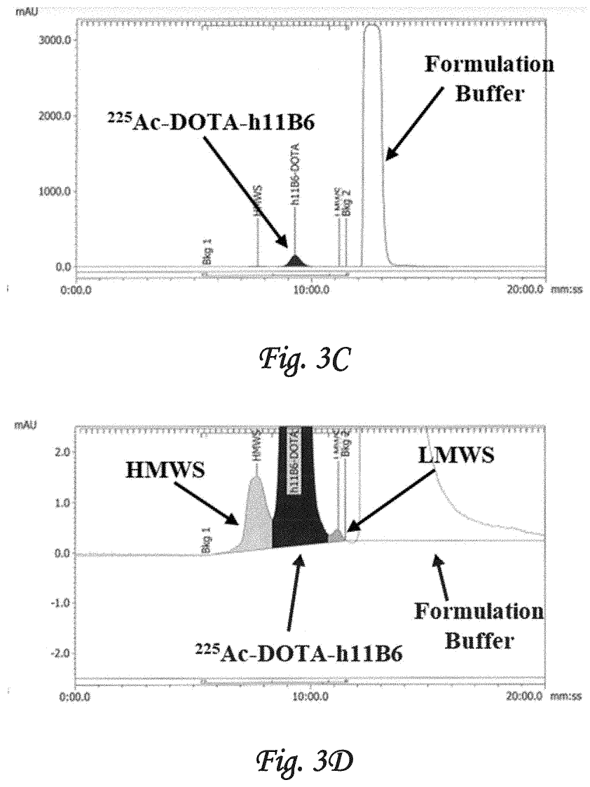

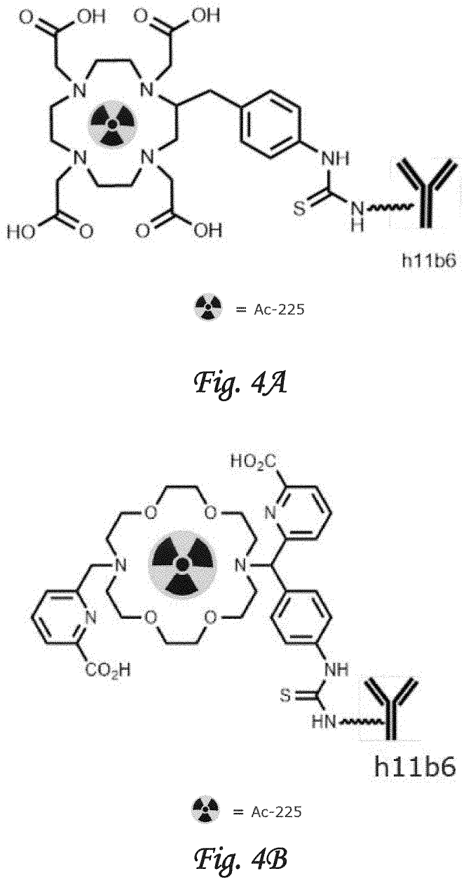

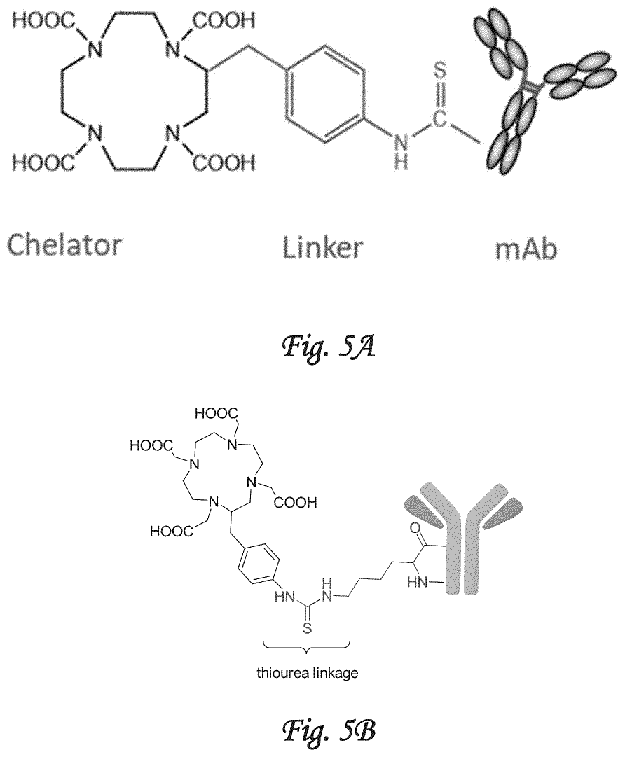

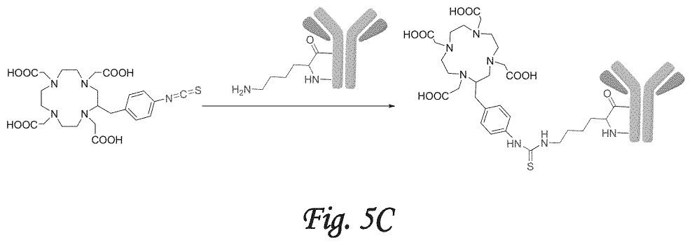

The cryoprotectant, sucrose, was eliminated from Formulation A due to the formation of secondary radiolytic degradation products from primary radiation. The form was modified from a frozen solid to a liquid solution. The elimination of sucrose, however, resulted in accelerated degradation of the ²²⁵Ac-DOTA-h11B6 drug product, in particular, accelerated degradation of the h11B6 antibody. 0.5% w/v sodium ascorbate (vitamin C) was added as a sacrificial radioprotectant to attenuate degradation, which resulted in a “Formulation B” containing 0.5 mg/mL ²²⁵Ac-DOTA-h11B6 in 26.75 mM acetate, 0.5% w/v sodium ascorbate (vitamin C), and 0.04% polysorbate 20, pH 5.5. UV and Radio-HPLC chromatograms of this formulation were obtained. See, FIGS. 3A-3D. Degradation of the ²²⁵Ac-DOTA-h11B6 drug product was significantly reduced in Formulation B.

### Example 4: Manufacture of ²²⁵Ac-DOTA-h11B6 and Formulation “B” Comprising ²²⁵Ac-DOTA-h11B6

While this example describes manufacture of drug product containing ²²⁵Ac-DOTA-h11B6, analogous methods may also be used for manufacture of a drug product containing ²²⁵Ac-TOPA-h11B6 in place of ²²⁵Ac-DOTA-h11B6.

This example describes processes for the manufacture of ²²⁵Ac-DOTA-h11B6 drug product and a solution containing the drug product. The antibody h11B6 may be prepared as described, for example, in U.S. Pat. No. 10,100,125, which is incorporated by reference herein. The antibody h11B6 may also be prepared using methods described in U.S. Pat. No. 9,873,891, which is incorporated by reference herein, using a CHO DG44 derived cell line and an hEF1α promoter double gene vector, commercially available from Fujifilm Diosynth Biotechnologies.

Following preculture and expansion, the cell culture may be clarified using known filtration techniques. The filtrate is concentrated and diafiltered to a target final concentration of 10 g/L in buffer (25 mM NaOAc, pH 5.5). The h11B6 is filtered through a 0.2-μm filter, filled into sterilized bags, and may be frozen at ≤−65° C. for long-term storage, prior to conjugation.

The thawed h11B6 is then diafiltered (buffer exchanged) to 50 mM Bicine, 120 mM NaCl, pH 8.5 for the subsequent conjugation of DOTA (1,4,7,10-tetraazacyclododecane-1,4,7,10-tetraacetic acid) to h11B6. The retentate from the prior step is transferred to a reactor and stirred while warming to 25° C. A solution of p-SCN-Bn-DOTA in water is prepared and added to the reactor. The reaction is maintained at 25° C. for 20 hours. The product of the conjugation reaction (DOTA-h11B6) is transferred directly to the retentate vessel for the final diafiltration with 25 mM NaOAc, pH 5.5. Next, DOTA-h11B6 conjugate intermediate is filtered through a 0.2-μm filter, filled into sterilized polycarbonate containers, and may be frozen at ≤−65° C. for long-term storage.

The conjugation reaction results in addition of multiple DOTA molecules to the epsilon amino group of lysine side chains of the h11B6 mAb. The conjugate-to-antibody ratio (CAR), which designates the number of DOTA molecules per h11B6 mAb molecule, can be measured by intact mass analysis using RP-HPLC with online mass analysis. Based on the molecular structure of p-SCN-Bn-DOTA, each DOTA residue adds 552 Da mass to the antibody, which can be readily detected by intact mass analysis. To reduce the sample complexity, DOTA-h11B6 conjugate intermediate was treated with PNGase F to remove N-linked glycans and carboxypeptidase B to remove C-terminal lysine residues. The average CAR of DOTA-h11B6 of 2.6 was calculated as a weighted average from all detected CAR species.

The ²²⁵Ac-DOTA-h11B6 drug product is produced in a continuous operation from the precursor, DOTA-h11B6 conjugate intermediate, which reacts with ²²⁵Actinium trichloride to generate an ²²⁵Actinium-radiolabeled drug substance with a target specific activity of ≥170 μCi/mg. The ²²⁵Ac-DOTA-h11B6 drug substance is synthesized, purified by PD-10 column purification, and formulated in situ. The four drug product presentations (50, 100, 150, and 200 μCi in 2 mg protein) are manufactured by blending of the ²²⁵Ac-DOTA-h11B6 drug substance with DOTA-h11B6 and reformulation buffer, as described below. The blended product presentations are individually sterile filtered and aseptically filled into the final patient vial.

An intermediate purification buffer (26.75 mM acetate, 0.04% Polysorbate 20, acetic acid, pH 5.5) is prepared for the final compounded purification buffer and can be stored at 2-8° C. for ≤30 days prior to use. Sodium ascorbate is added to the intermediate purification buffer and filtered through a 0.2-μm sterilizing filter into a sterile product holding vessel to produce the final compounded purification buffer (26.75 mM acetate, 0.5% (w/v) sodium ascorbate, 0.04% (w/v) polysorbate 20, acetic acid, pH 5.5).

The DOTA-h11B6 conjugate intermediate is thawed at room temperature. A solution of actinium trichloride is prepared by dissolving actinium trinitrate in 0.1 N hydrochloric acid (it is also possible to use sources of Ac-225 that are already in trichloride form). Actinium trichloride (800-1300 μCi) is incubated with 4.4 mg DOTA-h11B6 and sodium acetate buffer (pH adjustment with acetic acid, prepared ahead of time and stored≤6 months), pH adjusted to 6.5. ²²⁵Ac-DOTA-h11B6 is then purified on a PD-10 column pre-conditioned and eluted with the final purification buffer. After purification, the amount of radioactivity is measured.

In preparation to achieve the four dose amounts (50, 100, 150, and 200 μCi), the DOTA-h11B6 conjugate intermediate is reformulated to 0.5 mg/mL (26.75 mM acetate, 0.5% (w/v) sodium ascorbate, 0.04% (w/v) polysorbate 20, acetic acid pH 5.5) using the final reformulation buffer and filtered through a 0.2-μm sterilizing filter. ²²⁵Ac-DOTA-h11B6 is then dispensed into an intermediate vial to achieve the desired unit dose (50, 100, 150, and 200 μCi in 4 mL at the anticipated time of administration to a patient) and reformulated DOTA-h11B6 conjugate intermediate is added to a volume of 6.8 mL to produce the drug product. The drug product is then filtered through a 0.2-μm sterilizing filter and aseptically filled to a volume of 4.8 mL. The remaining drug product is also filtered through a 0.2-μm sterilizing filter and aseptically filled for release testing of the drug product. The drug product is immediately stored at 2-8° C.

Thus, the radiolabeled drug product ²²⁵Ac-DOTA-h11B6 is prepared as a sterile solution for intravenous injection and does not contain a preservative. The ²²⁵Ac-DOTA-h11B6 is available in four drug product (DP) unit doses: 50, 100, 150 and 200 μCi at anticipated time of administration to a patient.

The targeted compositions of the drug products are provided in Table 2 below. The compositions may alternatively be prepared with ²²⁵Ac-TOPA-h11B6 and TOPA-h11B6, instead of ²²⁵Ac-DOTA-h11B6 and DOTA-h11B6, respectively.

TABLE 2
Composition and concentration of the 50, 100, 150 and 200 μCi drug products

| | | | Target Amount | | Target | |
|---|---|---|---|---|---|---|
| Component | Quality Reference | Function | per Vial^{a} | | Concentration | |
| ²²⁵Ac-DOTA-h11B6^{b} and | N/A | Drug Substance | 2.4 ± 0.48 | mg^{c} | 0.5 ± 0.1 | mg/mL^{c} |
| DOTA h11B6^{c} | | | | | | |
| 50 μCi | | | 60.0 | μCi | 12.5 | μCi/mL |
| 100 μCi | | | 120.0 | μCi | 25.0 | μCi/mL |
| 150 μCi | | | 180.0 | μCi | 37.5 | μCi/mL |
| 200 μCi | | | 240.0 | μCi | 50 | μCi/mL |
| Sodium Acetate Trihydrate | USP/NF/Ph. Eur/JP | Buffer | 15.17 | mg | 3.16 | mg/mL |
| Acetic Acid | USP/NF/Ph. Eur/JP | Buffer | 1.01 | mg | 0.21 | mg/mL |
| Sodium Ascorbate | USP | Radioprotectant | 24 | mg | 5.0 | mg/mL |
| Polysorbate 20 | USP/NF/Ph. Eur/JP | Surfactant | 1.92 | mg | 0.40 | mg/mL |

| | | | | |
|---|---|---|---|---|
| Water for injection (WFI) | USP | Solvent | qs. | qs. |

^{a}The target fill volume (4.8 mL) includes a 0.8 mL overfill.
^{b}Target activity concentration at time of calibration.
^{c225}Ac-DOTA-h11B6 is combined with DOTA-h11B6 and reformulation buffer to produce the respective drug product unit doses, as described herein.

### Example 5A: DOTA-h11B6 Stability Study

This study was conducted to monitor DOTA-h11B6 Drug Substance Intermediate (10 mg/mL formulated in 25 mM acetate, pH adjusted to 5.5) attributes placed on stability under various environmental conditions and lengths of time. Study test articles were prepared by aliquoting Drug Substance Intermediate (DSI) into 20 mL Polycarbonate bottles at a fill volume of 9 mL.

### Study Parameters

| Stability Classification | Storage condition | Duration (Months) |
|---|---|---|
| Recommended | ≤−65° C. ° C. | 48 |
| Accelerated | −40° C. ± 2° C. | 6 |
| Stressed | 5° C. ± 2° C. | 6 |
| Stressed | 25° C. ± 2° C. | 1 |

Stability Study Results

The stability results for DOTA-h11B6 DSI held under recommended, accelerated, and two stressed conditions are listed below. At all-time points for DSI held at recommended storage conditions, all test parameter result values observed per assay study exceeded the criteria consistent with the most preferred embodiment of the stability when held after storage for about 48 months or more and at a temperature of about −65° C., after storage for about 6 months or more and at a temperature of about −40° C., after storage for about 6 months or more and at a temperature of about 5° C., and/or after storage for about 4 years or more and at a temperature of about −65° C. Of particular note, DSI held at accelerated conditions (−40° C.) for 6 months and DSI held at stressed conditions (5° C.) for 6 months showed results consistent with the preferred embodiment of the stability when held at −65° C. for about 4 years or more.

Results for DOTA-h11B6 DSI held at stressed conditions (25° C.) for 1 month showed the below rates of degradation for Drug Substance Intermediate exposed to this stressed storage conditions.

### −65° C. Data

TABLE A
Stability Results for DOTA-h11B6 Drug Substance Intermediate (DSI) at −65° C.

| | |
|---|---|
| Protein Conc | SEC-HPLC |

| Months | Appearance | (mg/mL) | pH | Main Peak (%) | HMW (%) | LMW: (%) |
|---|---|---|---|---|---|---|
| 0 | Clear and colourless Liquid, free of | 10.0 | 5.5 | 98.7 | 1.2 | 0.1 |
| | visible particulates | | | | | |
| 3 | Clear and colourless Liquid, free of | 10.0 | 5.4 | 98.6 | 1.2 | 0.2 |
| | visible particulates | | | | | |
| 6 | Clear and colourless Liquid, free of | 10.1 | 5.5 | 98.6 | 1.2 | 0.2 |
| | visible particulates | | | | | |
| 12 | Clear, Colourless Liquid 1 thread like | 10.1 | 5.5 | 98.5 | 1.1 | 0.3 |
| | particulates present | | | | | |
| 18 | Clear and colourless Liquid, free of | 10.1 | 5.5 | 98.7 | 1.2 | 0.2 |
| | visible particulates | | | | | |
| 24 | Slightly yellow, slightly opalescent | 10.1 | 5.4 | 98.7 | 1.1 | 0.1 |
| | liquid, free of visible particulates | | | | | |
| 36 | Clear and colourless Liquid, free of | 10.0 | 5.5 | 98.6 | 1.2 | 0.2 |
| | visible particulates | | | | | |
| 48 | Clear, Colourless Liquid, and free | 10.2 | 5.4 | 98.5 | 1.1 | 0.4 |
| | from particulates | | | | | |

cSDS

| | Reduced | | | | Non-reduced |
|---|---|---|---|---|---|
| Months | HC (%) | LC (%) | NGHC (%) | HC + LC (%) | Main IgG Purity (%) |
| 0 | 65.5 | 32.8 | 0.4 | 98.3 | 96.1 |
| 3 | 62.9 | 33.1 | 0.5 | 96.0 | 96.3 |
| 6 | 63.3 | 33.3 | 0.4 | 96.6 | 95.6 |
| 12 | 64.8 | 33.4 | 0.4 | 98.2 | 95.7 |
| 18 | 63.5 | 33.3 | 0.4 | 96.8 | 95.7 |
| 24 | 65.1 | 33.1 | 0.4 | 98.1 | 95.5 |
| 36 | 65.7 | 32.3 | 0.4 | 98.0 | 96.0 |
| 48 | 64.2 | 34.2 | 0.4 | 98.4 | 95.6 |

| | cIEF | | RP-HPLC |
|---|---|---|---|
| Months | Free mAh (μg/mg) | Mean pI (%) | residual DOTA (μg/mL) |
| 0 | 52 | 7.4 | 1.036 |
| 3 | 37 | 7.5 | 0.972 |
| 6 | 20 | 7.3 | 1.042 |
| 12 | 29 | 7.4 | 1.026 |
| 18 | 28 | 7.4 | 0.924 |
| 24 | 49 | 7.5 | 0.895 |
| 36 | 23 | 7.4 | 0.810 |
| 48 | 44 | NR | 0.929 |

SE-HPLC = Size Exclusion High Performance Liquid Chromatography;
HMW = High Molecular Weight;
LMW = Low Molecular Weight;
CE-SDS = Capillary Electrophoresis Sodium Dodecyl Sulfate;
LC = Light Chain;
HC = Heavy Chain;
NGHC = Non-Glycosylated Heavy Chain;
IgG = Immunoglobulin G;
CIEF = Cation Exchange;
NR = Not Reported

### −40° C. Data

TABLE B
Stability Results for DOTA-h11B6 Drug Substance Intermediate (DSI) at −40° C.

| | |
|---|---|
| Protein Conc. | SEC-HPLC |

| Months | Appearance | (mg/mL) | pH | Main Peak (%) | HMW (%) | LMW: (%) |
|---|---|---|---|---|---|---|
| 0 | Clear and colourless Liquid, free of | 10.1 | 5.5 | 98.5 | 1.3 | 0.3 |
| | visible particulates | | | | | |
| 3 | Clear and colourless Liquid, free of | 10.1 | 5.5 | 98.5 | 1.2 | 0.3 |
| | visible particulates | | | | | |
| 6 | Slightly opalescent, Colourless Liquid, | 10.1 | 5.5 | 98.6 | 1.3 | 0.2 |
| | No visible particulates present | | | | | |

cSDS

| | Reduced | | | | Non-reduced |
|---|---|---|---|---|---|
| Months | HC (%) | LC (%) | NGHC (%) | HC + LC (%) | Main IgG Purity (%) |
| 0 | 65.5 | 32.6 | 0.4 | 98.1 | 96.2 |
| 3 | 64.6 | 33.5 | 0.4 | 98.1 | 95.6 |
| 6 | 64.5 | 33.6 | 0.4 | 98.1 | 95.6 |

| | cIEF | | RP-HPLC |
|---|---|---|---|
| Months | Free mAb (μg/mg) | Mean pI (%) | residual DOTA (μg/mL) |
| 0 | 8 | 7.4 | 0.741 |
| 3 | 27 | 7.4 | 0.976 |
| 6 | 19 | 7.3 | 0.936 |

SE-HPLC = Size Exclusion High Performance Liquid Chromatography;
HMW = High Molecular Weight;
LMW = Low Molecular Weight;
CE-SDS = Capillary Electrophoresis Sodium Dodecyl Sulfate;
LC = Light Chain;
HC = Heavy Chain;
NGHC = Non-Glycosylated Heavy Chain;
IgG = Immunoglobulin G;
cIEF = capillary Isoelectric Focusing;
RP-HPLC = Reverse Phase High Performance Liquid Chromatography

### 5° C. Data

TABLE C
Stability Results for DOTA-h11B6 Drug Substance Intermediate (DSI) at 5° C.

| | |
|---|---|
| Protein Conc. | SEC-HPLC |

| Months | Appearance | (mg/mL) | pH | Main Peak (%) | HMW (%) | LMW: (%) |
|---|---|---|---|---|---|---|
| 0 | Clear and colourless Liquid, free of | 10.1 | 5.5 | 98.5 | 1.3 | 0.3 |
| | visible particulates | | | | | |
| 1 | Clear and colourless Liquid, free of | 10.2 | 5.5 | 98.1 | 1.5 | 0.3 |
| | visible particulates | | | | | |
| 3 | Clear and colourless Liquid, free of | 10.2 | 5.5 | 98.0 | 1.7 | 0.3 |
| | visible particulates | | | | | |
| 6 | Slightly opalescent, Colourless Liquid, | 10.4 | 5.5 | 97.8 | 2.0 | 0.2 |
| | No visible particulates present | | | | | |

cSDS

| | Reduced | | | | Non-reduced |
|---|---|---|---|---|---|
| Months | HC (%) | LC (%) | NGHC (%) | HC + LC (%) | Main IgG Purity (%) |
| 0 | 65.5 | 32.6 | 0.4 | 98.1 | 96.2 |
| 1 | 64.7 | 33.5 | 0.4 | 98.2 | 95.6 |
| 3 | 64.7 | 33.5 | 0.4 | 98.2 | 95.5 |
| 6 | 63.7 | 33.3 | 0.4 | 97.0 | 95.5 |

| | cIEF | | RP-HPLC |
|---|---|---|---|
| Months | Free mAb (μg/mg) | Mean pI (%) | residual DOTA (μg/mL) |
| 0 | 8 | 7.4 | 0.741 |
| 1 | 29 | 7.4 | 0.975 |
| 3 | 30 | 7.4 | 0.911 |
| 6 | 21 | 7.3 | 0.850 |

SE-HPLC = Size Exclusion High Performance Liquid Chromatography;
HMW = High Molecular Weight;
LMW = Low Molecular Weight;
CE-SDS = Capillary Electrophoresis Sodium Dodecyl Sulfate;
LC = Light Chain;
HC = Heavy Chain;
NGHC = Non-Glycosylated Heavy Chain;
IgG = Immunoglobulin G;
cIEF = capillary Isoelectric Focusing;
RP-HPLC = Reverse Phase High Performance Liquid Chromatography

### 25° C. Data

TABLE D
Stability Results for DOTA-h11B6 Drug Substance Intermediate (DSI) at 25° C.

| | |
|---|---|
| Protein Conc. | SEC-HPLC |

| Months | Appearance | (mg/mL) | pH | Main Peak (%) | HMW (%) | LMW: (%) |
|---|---|---|---|---|---|---|
| 0 | Clear, Colourless, | 10.1 | 5.5 | 98.5 | 1.3 | 0.3 |
| | particulates present | | | | | |
| 1 | Clear, Colourless Liquid, No | 10.2 | 5.5 | 97.8 | 1.8 | 0.4 |
| | visible particulates present | | | | | |

cSDS

| | Reduced | | | | Non-reduced |
|---|---|---|---|---|---|
| Months | HC (%) | LC (%) | NGHC (%) | HC + LC (%) | Main IgG Purity (%) |
| 0 | 65.5 | 32.6 | 0.4 | 98.1 | 96.2 |
| 1 | 64.5 | 33.5 | 0.4 | 98.0 | 95.2 |

| | cIEF | | RP-HPLC |
|---|---|---|---|
| Months | Free mAb (μg/mg) | Mean pI (%) | residual DOTA (μg/mL) |
| 0 | 8 | 7.4 | 0.741 |
| 1 | 29 | 7.4 | 0.308 |

SE-HPLC = Size Exclusion High Performance Liquid Chromatography;
HMW = High Molecular Weight;
LMW = Low Molecular Weight;
CE-SDS = Capillary Electrophoresis Sodium Dodecyl Sulfate;
LC = Light Chain;
HC = Heavy Chain;
NGHC = Non-Glycosylated Heavy Chain;
IgG = Immunoglobulin G;
cIEF = capillary Isoelectric Focusing;
RP-HPLC = Reverse Phase High Performance Liquid Chromatography

### Example 5B: ¹¹¹In-DOTA-h11B6 Stability Study

This study was conducted to monitor ¹¹¹In-DOTA-h11B6 Drug Product (DP) attributes placed on stability under recommended storage conditions. Study test articles were prepared by filling Drug Product through the septum of pre-stoppered, capped, and crimp-sealed 10R borosilicate vials.

### Study Parameters

| Stability Classification | Storage condition | Duration (Hours) |
|---|---|---|
| Recommended | −40° C. ± 10° C. | 72 |
| Accelerated | 5° C. ± 2° C. | 72 |

Stability Study Results

The stability results for ¹¹¹In-DOTA-h11B6 DP held under recommended and accelerated conditions are listed below. At all-time points for DP held at recommended storage conditions, all test parameter result values observed per assay study exceeded the criteria consistent with the most preferred embodiment of the stability when held after storage for about 72 hours or more and at a temperature of about −40° C., and/or after storage for about 72 hours or more and at a temperature of about 5° C.

Results for DP held at accelerated (5° C.) for 72 hours showed results consistent with the preferred embodiment of the stability when held at −40° C. for about 72 hours or more.

### 40° C. Data

TABLE A1
Stability Results for ¹¹¹In-DOTA-h116B6 stored at −40° C.

| | | Protein Purity by | | Protein | |
|---|---|---|---|---|---|
| | | UV-HPLC | | Concentration | Immunoreactivity |
| Hours | Appearance | Main Peak (%) | pH | (mg/mL) | Binding (%) |
| 0 | Clear and free of | 98 | 5.51 | 0.5 | 95.36 |
| | particles | | | | |
| 48 | Clear and free of | 97 | 5.57 | 0.5 | 92.82 |
| | particles | | | | |
| 72 | Clear and free of | 96 | 5.54 | 0.5 | 91.79 |
| | particles | | | | |

| | ¹¹¹In-h11B6 | ¹¹¹In-h116B6 Radiochemical Purity | | |
|---|---|---|---|---|
| | Radiochemical ID | Main | | |
| Hours | Relative Retention Time | Component (%) | HMWS (%) | LMWS(%) |
| 0 | 1.0 | 99.4 | 0.6 | ND |
| 48 | 1.0 | 98.1 | 1.9 | ND |
| 72 | 1.0 | 97.7 | 2.3 | ND |

ND = Not Detected,
HMWS = High Molecular, Weight Species,
LMWS = Low Molecular Weight Species

### 5° C. Data

TABLE A2
Stability Results for ¹¹¹In-DOTA-h116B6 stored at 5° C.

| | | Protein Purity by | | Protein | |
|---|---|---|---|---|---|
| | | UV-HPLC | | Concentration | Immunoreactivity |
| Hours | Appearance | Main Peak (%) | pH | (mg/mL) | Binding (%) |
| 0 | Clear and free of | 98 | 5.51 | 0.5 | 95.36 |
| | particles | | | | |
| 48 | Clear and free of | 95 | 5.62 | 0.5 | 95.32 |
| | particles | | | | |
| 72 | Clear and free of | 95 | 5.52 | 0.5 | 90.38 |
| | particles | | | | |

| | ¹¹¹In-h11B6 | ¹¹¹In-h116B6 Radiochemical Purity | | |
|---|---|---|---|---|
| | Radiochemical ID | Main | | |
| Hours | Relative Retention Time | Component (%) | HMWS (%) | LMWS(%) |
| 0 | 1.0 | 99.4 | 0.6 | ND |
| 48 | 1.0 | 97.5 | 2.5 | ND |
| 72 | 1.0 | 96.6 | 3.4 | ND |

HMWS = High Molecular, Weight Species,
LMWS = Low Molecular Weight Species,
μCi = microCurie;
ND = Not Detected

### Example 5C: ²²⁵Ac-DOTA-h11B6 Stability Study

This study was conducted to monitor ^{22S}Ac-DOTA-h11B6 Drug Product (50 μCi and 200 μCi) attributes placed on stability under recommended storage conditions. Study test articles were prepared by filling Drug Product through the septum of pre-sealed 10R cyclic olefin polymer vials.

### Study Parameters

| Stability Classification | Storage condition | Duration (Hours) |
|---|---|---|
| Recommended | 2-8° C. | 96 |

Stability Study Results

The stability results for ²²⁵Ac-DOTA-h11B6 DP held under recommended storage conditions, are listed below. At all-time points for DP held at recommended storage conditions, all test parameter result values observed per assay study exceeded the criteria consistent with the most preferred embodiment of the stability when held after storage for about 96 hours.

### 2-8° C. Data

TABLE B1
Stability Results for ²²⁵Ac-DOTA-h116B6 stored at 2-8° C. 50 μCi

| | | | | Radioactive | |
|---|---|---|---|---|---|
| | | | Protein | Concentration at Time | |
| | | | Concentration | of Calibration | Immunoreactivity |
| Hours | Appearance | pH | (mg/mL) | (μCi/mL) | (%) |
| 0 | Clear, Colorless, free of | 5.7 | 0.51 | 12.0 | 103 |
| | visible particulates | | | | |
| 72 | Clear, Colorless, free of | 5.7 | 0.51 | 12.12 | 90 |
| | visible particulates | | | | |
| 96 | Clear, Colorless, free of | 5.7 | 0.49 | 11.37 | 90 |
| | visible particulates | | | | |
| | | | | Radiochemical Purity | |
| | Protein Purity by SEC | | | Main | |
| | Main | HMWS | | Component | Sum of Impurities |
| Hours | Component (%) | (%) | LMWS(%) | (%) | (%) |
| 0 | 98.6 | 1.3 | 0.1 | 96 | 4 |
| 72 | 98.5 | 1.4 | 0.1 | 94 | 6 |
| 96 | 98.0 | 1.7 | 0.3 | 95 | 5 |

SEC = Size exclusion chromatography,
HMWS = High Molecular, Weight Species,
LMWS = Low Molecular Weight Species,
μCi = microCurie

TABLE B2
Stability Results for ²²⁵Ac-DOTA-h116B6 stored at 2-8° C. 200 μCi

| | | | | Radioactive | |
|---|---|---|---|---|---|
| | | | Protein | Concentration at Time | |
| | | | Concentration | of Calibration | Immunoreactivity |
| Hours | Appearance | pH | (mg/mL) | (μCi/mL) | (%) |
| 0 | Clear, Colorless, free of | 5.7 | 0.51 | 47.5 | 94 |
| | visible particulates | | | | |
| 72 | Clear, Colorless, free of | 5.7 | 0.51 | 46.9 | 94 |
| | visible particulates | | | | |
| 96 | Clear, Colorless, free of | 5.7 | 0.51 | 46.0 | 94 |
| | visible particulates | | | | |
| | | | | Radiochemical Purity | |
| | Protein Purity by SEC | | | Main | |
| | Main | HMWS | | Component | Sum of Impurities |
| Hours | Component (%) | (%) | LMWS(%) | (%) | (%) |
| 0 | 98.1 | 1.7 | 0.2 | 96 | 4 |
| 72 | 97.8 | 2.1 | 0.2 | 95 | 5 |
| 96 | 97.7 | 2.2 | 0.1 | 93 | 7 |

SEC = Size exclusion chromatography,
HMWS = High Molecular, Weight Species,
LMWS = Low Molecular Weight Species,
μCi = microCurie

### Example 6: Use of an Actinium-225-Labeled Antibody Targeting Human Kallikrein-2 (hK2) for Advanced Prostate Cancer (Study IDs: NCT04644770; 69086420PCR1001)

This example describes a first-in-human Phase 1 study to evaluate the safety, pharmacokinetics, pharmacodynamics, and preliminary antitumor activity of ²²⁵Ac-DOTA-h11B6 administered to adult patients with mCRPC who have disease progression on or following AR-targeted therapy. Formulation B described in Examples 3 and 4 is administered to patients in the Phase 1 trial. As discussed herein, ²²⁵Ac-DOTA-h11B6 is an hK2-specific monoclonal antibody, h11B6, labeled with DOTA and chelated to the α-particle-emitting radionuclide ²²⁵Ac, and is a radioimmunotherapy targeted to the hK2 antigen. It is noted that patient cohorts of this study will be administered ²²⁵Ac-TOPA-h11B6, in place of ²²⁵Ac-DOTA-h11B6, according to analous clinical methods described in this example and using an analogous pharmaceutical composition.

The primary objectives are to determine the safety and recommended Phase 2 dose(s) (RP2Ds) of ²²⁵Ac-DOTA-h11B6 and to evaluate the incidence, duration, and severity of adverse events, including dose-limiting toxicity (DLT). Secondary objectives and endpoints will evaluate the preliminary antitumor activity and provide a further understanding of the pharmacology of ²²⁵Ac-DOTA-h11B6. See, e.g., Table 3.

TABLE 3

| | |
|---|---|
| Objectives | Endpoints |

Primary

| | |
|---|---|
| Part 1 (Dose Escalation) | Part 1 (Dose Escalation) |
| Determine RP2Ds of ²²⁵Ac-DOTA-h11B6 | |
| | Incidence, duration, and severity of adverse |
| | events, including dose-limiting toxicity |
| Part 2 (Dose Expansion) | Part 2 (Dose Expansion) |
| Determine safety at the RP2D(s) | Incidence and severity of adverse events |

Secondary

| | |
|---|---|
| Assess the preliminary antitumor activity | PSA response |
| | Overall response rate (ORR) according to |
| | response criteria of Prostate Cancer |
| | Working Group 3 (PCWG3) |
| Assess the pharmacokinetics and | Serum radioactivity-time profiles and |
| immunogenicity | pharmacokinetic parameters for |
| | ²²⁵Ac-DOTA-h11B6 |
| | Presence of anti-²²⁵Ac-DOTA-h11B6 antibodies |

Exploratory
Explore the relationships between pharmacokinetics, pharmacodynamics, adverse event
profile, and antitumor activity.
Rate of PSA decline following single-dose and radiographic response.

²²⁵Ac-DOTA-h11B6 will be administered to adult males≥18 years with mCRPC who have had prior exposure to at least one novel AR-targeted therapy. Administration of ²²⁵Ac-DOTA-h11B6 will be conducted in 2 parts: dose escalation (Part 1) and dose expansion (Part 2).

Response to treatment will be assessed according to the response criteria of PCWG3.

Blood samples will be collected to characterize the pharmacokinetics of serum radioactivity and the concentration of h11B6 antibody, and to characterize the presence of anti-drug antibodies of ²²⁵Ac-DOTA-h11B6.

The safety of ²²⁵Ac-DOTA-h11B6 will be assessed by physical examinations, Eastern Cooperative Oncology Group (ECOG) performance status, electrocardiograms, clinical laboratory tests, vital signs, and adverse event monitoring. Echocardiogram or multigated acquisition scans will be assessed at screening; subsequent evaluations will be conducted if clinically indicated. The severity of adverse events will be assessed using National Cancer Institute Common Terminology Criteria for Adverse Events (Version 5.0). Concomitant medication usage will be recorded.

Dose escalation decisions will be supported by a modified continual reassessment method (mCRM) based on a Bayesian logistic regression model (BLRM) with overdose control (EWOC).

Inclusion Criteria Include the Following:

Each potential patient must satisfy all of the following criteria:

| | |
|---|---|
| 1 | Histologic: mCRPC with histologic confirmation of adenocarcinoma. |
| | Adenocarcinoma with small-cell or neuroendocrine features is allowed |
| 2 | Must have had prior exposure to at least one novel androgen receptor (AR) |
| | targeted therapy (example, abiraterone acetate, enzalutamide, apalutamide, darolutamide); |
| | prior taxane or other chemotherapy is acceptable but not required |
| 3 | Treatment with other agents for prostate cancer, if received, must have been |
| | discontinued greater than or equal to (≥) 2 weeks prior to first dose. |
| 4 | Adequate organ functions as reflected in laboratory parameters. |
| 5 | ECOG performance status of 0 or 1 |

Exclusion Criteria Include the Following:

Any potential patient who meets any of the following criteria will be excluded:

| | |
|---|---|
| 1 | Part 1: Prior treatment with radium Xofigo (Ra 223 dichloride), strontium, or |
| | samarium therapy or radioconjugate therapy |
| 2 | Known history of myelodysplastic syndrome, leukemia, or hematological |
| | malignancy with features suggestive of myelodysplastic syndrome/acute myeloid |
| | leukemia at any timepoint |
| 3 | Toxicity from prior anticancer therapy has not resolved to baseline levels or to |
| | Grade less than or equal to ≤1 (except alopecia, radiation tissue fibrosis, or peripheral neuropathy) |
| 4 | Known allergies, hypersensitivity, or intolerance to ²²⁵Ac-DOTA-h11B6 or its |
| | excipients and protein therapeutics |
| 5 | Active or chronic hepatitis B or hepatitis C infection |

A. Part 1: Dose Escalation

Participants will receive intravenous (IV) injection of ²²⁵Ac-DOTA-h11B6 with one or multiple doses at the amounts described below.

In Part 1, 50 μCi/2 mg ²²⁵Ac-DOTA-h11B6 will be administered to the first dose escalation cohort. After the DLT evaluation in this initial cohort has been conducted, dose escalation to the next dose level of radioactive ²²⁵Ac-DOTA-h11B6 will be based on the review of all available additional data including, but not limited to, pharmacokinetic, pharmacodynamic, safety, and preliminary antitumor activity.

Table 4 shows the planned (provisional) dose escalation schedule to illustrate a possible dose escalation pathway, which includes doses above 200 μCi (e.g., 300 μCi or higher). Intermediate dose-level increments are possible to ensure the safety of study participants. The initial cohort will receive a radioactivity amount of 50 μCi ²²⁵Ac-DOTA-h11B6. Escalation will initially occur in 50 μCi increments. A dosing interval of one dose every 8 weeks will be used. The starting antibody (h11B6) mass amount is 2 mg with the possibility of increase up to 10 mg. Initially, the antibody mass dose will be kept constant with increasing radioactivity across cohorts.

TABLE 4
Dose Escalation Schedule

| | Dose | Maximum increment |
|---|---|---|
| Dose Level | (μCi) | from previous dose |
| Dose Level 1 | 50 μCi | Starting dose |
| Dose Level 2 | 100 μCi | 100% |
| Dose Level 3 | 150 μCi | 50% |
| Dose Level 4 | 200 μCi | 33% |
| Dose Level 5 | 300 μCi | 50% |

There are two components to the final drug product that will be administered to participants in this study: the ²²⁵Ac-DOTA-h11B6 and the unlabeled DOTA-h11B6 antibody. The two components may be pre-mixed in a single vial. The two components will be provided for each participant visit at the prescribed radioactivity dose, and a total antibody mass amount of between 2 and 10 mg. See, Table 5.

TABLE 5
²²⁵Ac-DOTA-h11B6 Radiotherapy Administration

| | ²²⁵Ac-DOTA-h11B6 (Radiolabeled | Naked h11B6 antibody (Non- |
|---|---|---|
| | Monoclonal Antibody | radiolabeled Antibody |
| Study drug: | with DOTA Linker) | with DOTA Linker) |
| Route of | IV injection | IV injection |
| administration | | |
| Unit dose | 50, 100, 150, or 200 μCi per 4 mL with | 10 mg/mL |
| strength(s)/ | protein concentration of 2 mg/4 mL. | |
| Dosage levels | Doses above 200 μCi will have a total | |
| | volume of 8 mL with protein | |
| | concentration of 2 mg/4 mL. | |
| Dosage | Refrigerated liquid | Refrigerated liquid |
| formulation | | |

| | |
|---|---|
| Schedule of | Once every 8 weeks for up to 4 doses; additional doses may be considered |
| administration | after discussion with the sponsor. |
| Dosing | The DOTA-mAb and radioconjugate ²²⁵Ac-DOTA-h11B6 may be pre- |
| instructions | mixed in a single vial. The two components will be provided for each |
| | participant visit at the prescribed radioactivity dose, and a total antibody |
| | mass amount of between 2 and 10 mg. |

The 225Ac-DOTA-h11B6 radioactive investigational product is a single use, sterile, refrigerated solution for injection in a cyclic olefin polymer vial closed with a latex free stopper and aluminum seal. The 225Ac-DOTA-h11B6 is formulated in 26.75 mM acetate, 0.5% sodium ascorbate, and 0.04% polysorbate 20 in sterile water at pH 5.5. The investigational product is clear, colorless to slightly yellow, and free of visible particulate matter. The 225Ac-DOTA-h11B6 vials are stored refrigerated in the temperature range of 2-8° C. and protected from light. The drug product does not contain any preservatives and is designed for single-use only. The vial supplied to the clinic contains an overfill of 0.8 mL (total 4.8 mL) to allow a final dose withdrawal of 4.0±0.4 mL, dependent upon the actual time of administration. The radioactive concentration of 225Ac-DOTA-h11B6 is initially targeted for 50, 100, 150, or 200 μCi in 4 mL (2 mg), followed by doses above 200 μCi (e.g., 300 which will have a total volume of 8 mL with protein concentration of 2 mg/4 mL, so 4 mg total protein, at the anticipated time of administration). Intermediate dose-level increments are possible to ensure the safety of study participants. As noted above, an analogous investigational product for this study will comprise ²²⁵Ac-TOPA-h11B6, in place of ²²⁵Ac-DOTA-h11B6, for additional patient cohorts.

Dose escalation will be supported using an adaptive dose escalation strategy guided by the modified continual reassessment method based on a BLRM with EWOC.

The RP2D(s) will be determined after review of all available pharmacokinetic, pharmacodynamic, safety, and efficacy data. Once the RP2D(s) have been determined, patients will be treated to confirm the safety, pharmacokinetics, pharmacodynamics, and preliminary antitumor activity of ²²⁵Ac-DOTA-h11B6 at the RP2D(s) in Part 2.

B. Part 2: Dose Expansion

In Part 2, the RP2D(s) of ²²⁵Ac-DOTA-h11B6, as determined in Part 1, will be administered to patients in one or more cohorts.

All adverse events and adverse events fulfilling the criteria of DLT, will be reviewed and confirmed. Adverse events will be evaluated according to NCI CTCAE Version 5.0. Criteria for DLT are outlined in Table 6.

As noted above, patient cohorts of this study will be administered ²²⁵Ac-TOPA-h11B6, instead of ²²⁵Ac-DOTA-h11B6, according to analogous clinical methods described in this example.

TABLE 6
Dose-limiting Toxicity Criteria ^{a}
Hematologic Toxicity

| | |
|---|---|
| Neutrophil count decreased | Febrile neutropenia |
| | Neutropenia: Grade 4 for >5 days |
| Platelet count decreased | Grade ≥3 thrombocytopenia with bleeding or Grade 4 |
| | thrombocytopenia of any duration |
| Any hematological toxicity | Grade 5 |

Non-hematological Toxicity
Any non-hematological toxicity of Grade ≥3, except for the following ^{c}:
Grade 3 fatigue, fever, constipation, or diarrhea lasting <7 days with best supportive care
Grade 3 nausea or vomiting lasting ≤48 hours that resolves to Grade ≤1 either spontaneously
or with best supportive care
Grade ≥3 ALT or AST that resolves to Grade ≤1 or baseline within 7 days, unless criteria for
Hy’s law are met ^{b}
Isolated Grade ≥3 ALP^{a} or GGT increase that returns to Grade ≤1 or baseline within 7 days
Grade ≥3 lipase or amylase increase not associated with clinical or radiological evidence of
pancreatitis
Grade ≥3 electrolyte abnormalities ^{c} that last ≤72 hours resolving spontaneously or with best
supportive care
^{a}. Unless unequivocally due to the underlying malignancy or an extraneous cause.
^{b}. Hy’s Law criteria defined as ALT or AST value ≥3 × ULN, total bilirubin ≥2 × ULN, and ALP ≤2 × ULN; with no alternative etiology. For patients with baseline Grade 2 elevation of AST or ALT due to liver metastasis, ALT or AST >3 × baseline or AST or ALT >8 × ULN, whichever is lower, combined with total bilirubin >2 × baseline and >2 × ULN will be considered meeting Hy’s law.
^{c}. Any chemistry abnormalities Grade ≥3 occurring during the DLT period must be reassessed to confirm the grade and resolution to Grade ≤2.

Outcome measures are provided in Table 7.

TABLE 7
Outcome Measures

| | Time | |
|---|---|---|
| Outcome Measure | Frame | Description |
| Part 1 and Part 2: | Up to 2 | An AE is any untoward medical occurrence in a |
| Number of patients | years and 4 | patient that does not necessarily have a causal |
| with AEs as a | months | relationship with the pharmaceutical/biological agent. |
| measure of safety | | |
| and tolerability | | |
| Part 1: Number of | Up to 2 | Number of patients with DLT will be assessed. The |
| patients with DLT | years and 4 | DLTs are specific adverse events and are defined as |
| | months | any of the following: high grade non-hematologic |
| | | toxicity, or hematologic toxicity. |
| Part 1 and Part 2: | Up to 2 | Severity will be graded according to the NCI CTCAE |
| Number of patients | years and 4 | version 5.0. Severity scale ranges from Grade 1 (Mild) |
| with AEs by | months | to Grade 5 (Death). Grade 1 = Mild, Grade 2 = |
| severity | | Moderate, Grade 3 = Severe, Grade 4 = Life- |
| | | threatening and Grade 5 = Death related to adverse event. |

Secondary outcome measures are provided in Table 8.

TABLE 8
Secondary Outcome Measures

| | Time | |
|---|---|---|
| Outcome Measure | Frame | Description |
| Percentage of patients | Week 12 | PSA response rate is defined as the |
| with PSA response | | percentage of patients with a decline of |
| | | PSA of 50% or more from baseline at Week 12. |
| Overall Response | Up to 2 | ORR is defined as the percentage of patients |
| Rate (ORR) | years and 4 | who have a partial response (PR) or better |
| | months | according to Response Evaluation Criteria in |
| | | Solid Tumors (RECIST) version 1.1 without |
| | | evidence of bone progression according to PCWG3. |
| Cₘₐₓ of ²²⁵Ac-DOTA-h11B6 | Up to 2 | Cmax is defined as the maximum |
| | years and 4 | observed serum concentration/ |
| | months | radioactivity of ²²⁵Ac-DOTA-h11B6. |
| Tₘₐₓ of ²²⁵Ac-DOTA-h11B6 | Up to 2 | Tmax is defined as time to reach |
| | years and 4 | maximum observed serum concentration/ |
| | months | radioactivity of ²²⁵Ac-DOTA-h11B6. |
| AUC₀₋ₜ of ²²⁵Ac-DOTA-h11B6 | Up to 2 | AUC₀₋ₜ is defined as the area under the |
| | years and 4 | serum concentration-time curve from time |
| | months | zero to t of ²²⁵Ac-DOTA-h11B6. |
| Number of patients | Up to 2 | Number of patients with anti-²²⁵Ac- |
| with anti-²²⁵Ac-DOTA- | years and 4 | DOTA-h11B6 antibodies will be assessed |
| h11B6 antibodies | months | to evaluate the potential immunogenicity. |

Interim Clinical Results

For 23 participants with metastatic castration-resistant prostate cancer (mCRPC) that were dosed ²²⁵Ac-DOTA-h11B6 across 4 radioactivity dose levels of 50, 100, 150 and 200 μCi in the 69/086,420PCR1001 study, the median number of doses received was 2 doses (range: 1 to 6), and the median treatment duration was 1.87 months (range: 1 to 10.8). No dose-limiting toxicities (DLT) were reported at any of the 4 radioactivity dose levels.

For those participants, the most frequently reported (≥15%) treatment-emergent adverse events (TEAEs) were fatigue (39.1%), decreased appetite (34.8%), diarrhea (26.1%), anemia and thrombocytopenia (21.7% each), and nausea and leukopenia (17.4% each). Most of these commonly reported TEAEs are of Grade 1 or 2, with the exception of 1 participant at 50 μCi with Grade 3 fatigue, 1 participant at 150 μCi with Grade 4 thrombocytopenia and 2 participants (1 at 50 μCi and 1 at 200 μCi) with Grade 3 anemia. Treatment emergent serious adverse events (SAE) have been reported for 2 participants: hypokalemia for 1 participant at 100 μCi and hypocalcemia for 1 participant at 150 μCi. One (1) participant at 150 μCi was discontinued due to thrombocytopenia while all the other discontinued participants were due to progressive disease or other reasons. No dose reduction was necessary in any participant. No on-treatment deaths were observed. Signals of efficacy in these participants include, for example, PSA decreases of 50% or more from baseline in patients at radioactive doses greater than or equal to 100 uCi.

The disclosures of each patent, patent application, and publication cited or described in this document are hereby incorporated herein by reference, each in its entirety, for all purposes.

1. A method of treating cancer in a patient, the method comprising:
administering to the patient a therapeutically effective amount of a pharmaceutical composition comprising a radioconjugate and one or more pharmaceutically acceptable excipients, wherein:
the radioconjugate comprises at least one radiometal complex conjugated to an antibody, or an antigen binding fragment, with binding specificity for hK2,
the radiometal complex comprises a radiometal, and
the radiometal provides a targeted radioactivity from about 50 μCi to about 350 μCi per dose of the pharmaceutical composition at the time of dosing.

2. The method according to claim 1, wherein the radioconjugate comprises at least one radiometal complex conjugated to an antibody with binding specificity for hK2.

3. The method according to claim 2, wherein the antibody comprises a heavy chain variable region comprising the amino acid sequences of SEQ ID NO:1 and SEQ ID NO:2 and SEQ ID NO:3; and a light chain variable region comprising the amino acid sequences of SEQ ID NO:4 and SEQ ID NO:5 and SEQ ID NO:6.

4. The method according to claim 2, wherein the antibody comprises a heavy chain variable region (VH) having at least 80%, at least 85%, at least 90%, at least 95%, or at least 98% sequence identity to the amino acid sequence of SEQ ID NO: 8, and a light chain variable region (VL) having at least 80%, at least 85%, at least 90%, at least 95%, or at least 98% sequence identity to the amino acid sequence of SEQ ID NO: 9.

5. The method according to claim 2, wherein the antibody comprises a heavy chain variable region (VH) comprising the amino acid sequence of SEQ ID NO: 8, and a light chain variable region (VL) comprising the amino acid sequence of SEQ ID NO: 9.

6. The method according to claim 2, wherein the antibody comprises a heavy chain constant region having at least 80%, at least 85%, at least 90%, at least 95%, or at least 98% sequence identity to the amino acid sequence of SEQ ID NO: 10, and a light chain constant region having at least 80%, at least 85%, at least 90%, at least 95%, or at least 98% sequence identity to the amino acid sequence of SEQ ID NO: 11.

7. The method according to claim 2, wherein the antibody comprises a heavy chain constant region comprising the amino acid sequence of SEQ ID NO: 10, and a light chain constant region comprising the amino acid sequence of SEQ ID NO: 11.

8. The method according to claim 2, wherein the antibody comprises a heavy chain having at least 80%, at least 85%, at least 90%, at least 95%, or at least 98% sequence identity to the amino acid sequence of SEQ ID NO: 12, and a light chain having at least 80%, at least 85%, at least 90%, at least 95%, or at least 98% sequence identity to the amino acid sequence of SEQ ID NO: 13.

9. The method according to claim 2, wherein the antibody comprises a heavy chain having the amino acid sequence of SEQ ID NO: 12, and a light chain having the amino acid sequence of SEQ ID NO: 13.

10. The method according to claim 1, wherein the radiometal is selected from the group consisting of ²²⁵Ac, ¹¹¹In, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th.

11. The method according to claim 1, wherein the radiometal is ²²⁵Ac.

12. The method according to claim 1, wherein the radiometal complex comprises a chelator that is selected from the group consisting of 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), and derivatives thereof.

13. The method according to claim 1, wherein the radiometal complex comprises a chelator that is DOTA.

14. The method according to claim 1, wherein the radiometal complex comprises ²²⁵AC chelated to DOTA.

15. The method according to claim 1, wherein the radioconjugate comprises the radiometal chelated to a compound of formula (V) 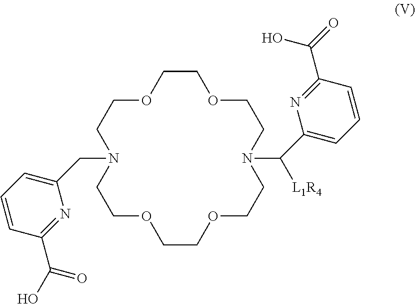
or a pharmaceutically acceptable salt thereof, wherein:
L₁ is absent or a linker; and
R₄ is the antibody.

16. The method according to claim 2, wherein the radiometal is ²²⁵Ac and the radiometal provides a targeted specific activity from about 50 μCi to about 350 μCi per between about 2 mg and about 10 mg of total antibody.

17.-31. (canceled)

32. The method according to claim 1, wherein the one or more pharmaceutically acceptable excipients comprise one or more radioprotectants.

33. The method according to claim 32, wherein the one or more radioprotectants comprise sodium ascorbate, gentisic acid, or a combination thereof.

34. The method according to claim 32, wherein the one or more radioprotectants comprise sodium ascorbate.

35. The method according to claim 32, wherein the one or more radioprotectants comprise gentisic acid.

36.-56. (canceled)

57. The method according to claim 1, comprising administering the pharmaceutical composition to the patient between once every about 12 weeks and once every about 4 weeks.

58. (canceled)

59. (canceled)

60. The method according to claim 1, wherein the cancer is prostate cancer.

61.-72. (canceled)

73. The method according to claim 1 comprising administering the dose in a single administration to the patient.

74. The method according to claim 1 comprising administering the dose in multiple administrations of more than one sub-dose.

75. The method according to claim 74 comprising the administering the dose as two sub-doses.

76. A pharmaceutical composition comprising:
a radioconjugate and one or more pharmaceutically acceptable excipients, wherein:
the radioconjugate comprises at least one radiometal complex conjugated to an antibody, or an antigen binding fragment, with binding specificity for hK2, and
the radiometal complex comprises a radiometal.

77. The pharmaceutical composition according to claim 76, wherein the one or more pharmaceutically acceptable excipients comprise one or more radioprotectants.

78. The pharmaceutical composition according to claim 77, wherein the radioconjugate comprises at least one radiometal complex conjugated to an antibody with binding specificity for hK2.

79. The pharmaceutical composition according to claim 78, wherein the antibody comprises a heavy chain variable region comprising the amino acid sequences of SEQ ID NO:1 and SEQ ID NO:2 and SEQ ID NO:3; and a light chain variable region comprising the amino acid sequences of SEQ ID NO:4 and SEQ ID NO:5 and SEQ ID NO:6.

80. The pharmaceutical composition according to claim 78, wherein the antibody comprises a heavy chain variable region (VH) having at least 80%, at least 85%, at least 90%, at least 95%, or at least 98% sequence identity to the amino acid sequence of SEQ ID NO: 8, and a light chain variable region (VL) having at least 80%, at least 85%, at least 90%, at least 95%, or at least 98% sequence identity to the amino acid sequence of SEQ ID NO: 9.

81. The pharmaceutical composition according to claim 78, wherein the antibody comprises a heavy chain variable region (VH) comprising the amino acid sequence of SEQ ID NO: 8, and a light chain variable region (VL) comprising the amino acid sequence of SEQ ID NO: 9.

82. The pharmaceutical composition according to claim 78, wherein the antibody comprises a heavy chain constant region having at least 80%, at least 85%, at least 90%, at least 95%, or at least 98% sequence identity to the amino acid sequence of SEQ ID NO: 10, and a light chain constant region having at least 80%, at least 85%, at least 90%, at least 95%, or at least 98% sequence identity to the amino acid sequence of SEQ ID NO: 11.

83. The pharmaceutical composition according to claim 78, wherein the antibody comprises a heavy chain constant region comprising the amino acid sequence of SEQ ID NO: 10, and a light chain constant region comprising the amino acid sequence of SEQ ID NO: 11.

84. The pharmaceutical composition according to claim 78, wherein the antibody comprises a heavy chain having at least 80%, at least 85%, at least 90%, at least 95%, or at least 98% sequence identity to the amino acid sequence of SEQ ID NO: 12, and a light chain having at least 80%, at least 85%, at least 90%, at least 95%, or at least 98% sequence identity to the amino acid sequence of SEQ ID NO: 13.

85. The pharmaceutical composition according to claim 78, wherein the antibody comprises a heavy chain having the amino acid sequence of SEQ ID NO: 12, and a light chain having the amino acid sequence of SEQ ID NO: 13.

86. The pharmaceutical composition according to claim 77, wherein the radiometal is selected from the group consisting of ²²⁵Ac, ¹¹¹In, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi ²¹³Bi, ²²³Ra, ²⁵⁵Fm and ²²⁷Th.

87. The pharmaceutical composition according to claim 77, wherein the radiometal is ²²⁵AC.

88. The pharmaceutical composition according to claim 77, wherein the radiometal complex comprises a chelator that is selected from the group consisting of 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), and derivatives thereof.

89. The pharmaceutical composition according to claim 77, wherein the radiometal complex comprises a chelator that is DOTA.

90. The pharmaceutical composition according to claim 77, wherein the radiometal complex comprises ²²⁵AC chelated to DOTA.

91. The pharmaceutical composition according to claim 77, wherein the radioconjugate comprises the radiometal chelated to a compound of formula (V) 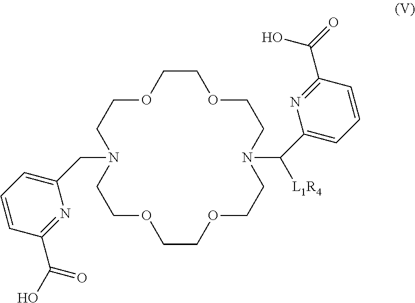
or a pharmaceutically acceptable salt thereof, wherein:
L₁ is absent or a linker; and
R₄ is the antibody.

92. The pharmaceutical composition according to claim 77, wherein the one or more radioprotectants comprise sodium ascorbate, gentisic acid, or a combination thereof.

93. The pharmaceutical composition according to claim 77, wherein the one or more radioprotectants comprise sodium ascorbate.

94. The pharmaceutical composition according to claim 77, wherein the one or more radioprotectants comprise gentisic acid.

95. The pharmaceutical composition according to claim 77, wherein the one or more pharmaceutically acceptable excipients further comprise one or more surfactants.

96. The pharmaceutical composition according to claim 95, wherein the one or more surfactants comprise polysorbate 20.

97. The pharmaceutical composition according to claim 77, wherein the one or more pharmaceutically acceptable excipients further comprise an acetate buffer.

98. The pharmaceutical composition according to claim 76 comprising the radioconjugate, sodium ascorbate, polysorbate 20, acetate buffer and water.

99. The pharmaceutical composition according to claim 76 comprising the radioconjugate, about 24-28 mM acetate, about 0.25-0.75% sodium ascorbate, and about 0.01-0.1% polysorbate 20 in water.

100. The pharmaceutical composition according to claim 76 comprising the radioconjugate, about 26.75 mM acetate, about 0.5% sodium ascorbate, and about 0.04% polysorbate 20 in water.

101. (canceled)

102. The pharmaceutical composition according to claim 76, wherein the pharmaceutical composition has a pH from about 5 to about 6 (e.g., about 5.5).

103. The pharmaceutical composition according to claim 76, wherein the pharmaceutical composition does not contain any preservatives.

104. The pharmaceutical composition according to claim 76, wherein the pharmaceutical composition does not contain any sucrose.

105. The pharmaceutical composition according to claim 76, wherein the pharmaceutical composition does not contain any monosaccharides, disaccharides, oligosaccharides or polysaccharides.

106. The pharmaceutical composition according to claim 76, wherein the pharmaceutical composition does not contain any monosaccharides or disaccharides.

107. The pharmaceutical composition according to claim 76 wherein the pharmaceutical composition does not contain any disaccharides.

108. The pharmaceutical composition according to claim 76, wherein the one or more pharmaceutically acceptable excipients consist of, or consist essentially of, acetate buffer, sodium ascorbate and polysorbate 20 in water.

109. The pharmaceutical composition according to claim 76, wherein the pharmaceutical composition is formulated for intravenous administration.

110. The pharmaceutical composition according to claim 76, wherein the pharmaceutical composition is stable at a temperature range of about 2-8° C. for at least 72 hours.

111. The pharmaceutical composition according to claim 77, wherein the radioconjugate comprises an average of from about 1 to about 4, or about 2 to about 3 chelator molecules conjugated to the antibody.

112. The pharmaceutical composition according to claim 77, wherein the radiometal is ²²⁵Ac and the radiometal provides a targeted specific activity from about 50 μCi to about 350 μCi per between about 2 mg and about 10 mg of total antibody at the time of dosing.

113.-127. (canceled)

128. The pharmaceutical composition according to claim 77 comprising a total amount of conjugate intermediate and the radioconjugate in an amount of about 0.1-1.0 mg/mL.

129. (canceled)

130. (canceled)

131. The pharmaceutical composition according to claim 77 further comprising non-radiolabeled antibody, wherein the non-radiolabeled antibody is the same antibody as the antibody conjugated to the radiometal complex.

132. The pharmaceutical composition according to claim 131, wherein the total amount of the conjugated antibody and the non-radiolabeled antibody does not exceed about 10 mg.

133. A method for treating cancer in a patient, the method comprising administering to the patient a therapeutically effective amount of the pharmaceutical composition of claim 76.

134. The method according to claim 133, comprising administering the pharmaceutical composition to the patient between once every about 12 weeks and once every about 4 weeks.

135. (canceled)

136. (canceled)

137. The method according to claim 133, wherein the cancer is prostate cancer.

138.-149. (canceled)


SEQUENCE LISTING (15 sequences)

SEQ ID NO: 1 - protein, 6 aa, Artificial Sequence
  Synthetic polypeptide
SDYAWN

SEQ ID NO: 2 - protein, 16 aa, Artificial Sequence
  Synthetic polypeptide
YISYSGSTTYNPSLKS

SEQ ID NO: 3 - protein, 8 aa, Artificial Sequence
  Synthetic polypeptide
GYYYGSGF

SEQ ID NO: 4 - protein, 15 aa, Artificial Sequence
  Synthetic polypeptide
KASESVEYFGTSLMH

SEQ ID NO: 5 - protein, 7 aa, Artificial Sequence
  Synthetic polypeptide
AASNRES

SEQ ID NO: 6 - protein, 9 aa, Artificial Sequence
  Synthetic polypeptide
QQTRKVPYT

SEQ ID NO: 7 - protein, 261 aa, Homo sapiens
  hK2 sequence
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 8 - protein, 117 aa, Artificial Sequence
  Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 9 - protein, 111 aa, Artificial Sequence
  Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 10 - protein, 330 aa, Artificial Sequence
  Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 107 aa, Artificial Sequence [sequence omitted]

SEQ ID NO: 12 - protein, 447 aa, Artificial Sequence
  Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 218 aa, Artificial Sequence
  Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 351 bp, Artificial Sequence
  Synthetic DNA
caggttcagctgcaggaaagcggacctggcttggtgaaacccagcgatacccttagcctgacatgtgctgtgtctggcaattccatcacttccgactatgcgtggaactggattcggcaaccaccgggaaaagggctcgagtggatagggtacatcagctattctggttcaaccacgtacaatccctcactgaagagtagggttaccatgtccagagacacctccaagaaccagttcagcctgaagctgagtagtgtgacagccgtagatacagccgtctattactgcgcaacagggtactactatggctctggcttttggggtcaaggaactctcgtcactgtgtcaagc

SEQ ID NO: 15 - DNA, 333 bp, Artificial Sequence
  Synthetic DNA
gacatagtgctcactcagagccctgatagcttggctgtcagtcttggggaaagagccaccatcaactgcaaagcgtccgaaagcgtcgagtatttcgggactagcctgatgcactggtatcagcagaaacccggacaaccgcctaagctgctgatctatgcagcctctaatcgcgaaagtggcgttccagacaggttttccggttctggatcaggcacagacttcaccctcacgatttcctcactgcaagctgaggatgtagccgtgtactactgtcagcagacacggaaagtgccctacacctttggtcagggcacaaagctggagattaag